(19) United States
(12) Patent Application Publication
(10) Pub. No.: US 2012/0122212 A1
(43) Pub. Date: May 17, 2012

(54) TGF-BETA PATHWAY INHIBITORS FOR ENHANCEMENT OF CELLULAR REPROGRAMMING OF HUMAN CELLS

(76) Inventors: Marica Grskovic, So. San Francisco, CA (US); John Dimos, So. San Francisco, CA (US); Berta Strulovici, So. San Francisco, CA (US); Adel Bakhtiarova, Woodside, CA (US); Weidong Wang, So. San Francisco, CA (US)

(21) Appl. No.: 13/203,780

(22) PCT Filed: Mar. 5, 2010
(86) PCT No.: PCT/US10/26451

§ 371 Date: Feb. 1, 2012

Related U.S. Application Data
(60) Provisional application No. 61/158,308, filed on Mar. 6, 2009.

(51) Int. Cl. C12N 5/071

(52) U.S. Cl. 435/371; 435/366; 435/372

(57) ABSTRACT

The present disclosure provides methods and compositions to enhance reprogramming in human cells. In some cases, the method includes contacting human cells with an inhibitor of the TGFβ pathway, for example a TGFβ receptor (TGFβR) inhibitor in combination with one or more induction factors.

### CROSS-REFERENCE

This application claims the benefit of U.S. Provisional Application No. 61/158,308, filed Mar. 6, 2009, which application is incorporated herein by reference in its entirety.

### BACKGROUND OF THE INVENTION

The discovery that adult human cells such as skin fibroblasts can be reprogrammed into a cell of greater potency without the aid of any embryonic tissue has had dramatic implications on the potential of cell replacement therapies. In the future, a routine approach to cell replacement therapy may be to take a cell from a patient, “induce” that cell to become a pluripotent cell, differentiate the pluripotent cell into any cell of interest and then transplant the cell back into the same patient, or a different patient. For example, a future routine therapy for Alzheimer's Disease may be to remove a skin cell from a patient, induce the cell to become pluripotent, and then coax it into becoming a neuron or a neural precursor cell suitable for transplantation. The generation of induced pluripotent stem cells (iPS cells) also has promising implications for drug screening and in vitro disease modeling, as panels of cells from genetically heterogeneous populations may be more easily created and studied.

Currently, the standard method of generating human iPS cells relies on the use of a viral vector to introduce certain factors associated with pluripotency, such as Oct3/4, Sox2, Klf4, and/or c-Myc, into a cell. Methods that employ small molecule compounds to increase or force the expression of one or more of these factors, or to enhance reprogramming by one or more of these factors may reduce variability during the production process and may result in the production of iPS cells that are more suitable for therapeutic applications.

### SUMMARY OF THE INVENTION

The present disclosure provides methods of enhancing reprogramming of human cells by induction factors (“IFs”) (also referred to as “reprogramming” factors).

Accordingly in one aspect provided herein is a method of increasing the potency of a human cell comprising contacting a plurality of human cells with a TGF-β receptor (TGFβR) inhibitor and forcing expression of one or more induction factors to obtain a plurality of human cells having increased potency.

In some cases, the plurality of human cells having increased potency have increased expression of one or more markers of pluripotency. Examples of such markers of pluripotency include, but are not limited to, one or more of alkaline phosphatase, SSEA-3, SSEA-4, TRA-1-60, TRA-1-81, Nanog, Oct-3/4, Sox2, GDF3, REX1, FGF4, ESG1, DPPA2, DPPA4, and hTERT. In some embodiments, the one or markers of pluripotency comprise alkaline phosphatase.

In some cases, the TGFβR inhibitor to be used in the just-mentioned method is a compound having the structure of Formula (II):

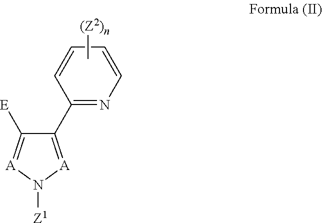

wherein,
E is selected from

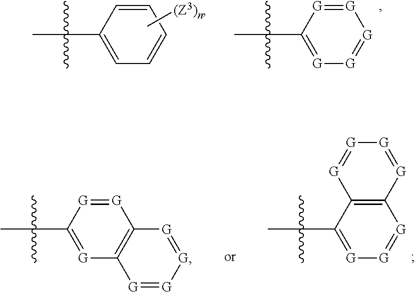

G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is H, CONHAr, or CSNHAr;
Ar is

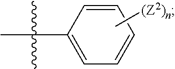

n is 0, 1, 2 or 3; and
each Z² is independently a C₁-C₈ straight, branched or cyclic hydrocarbon group, a C₁-C₆ alkoxy group, or, optionally, two Z² groups, together with the carbon atoms to which said Z² groups are attached, combine to form a cyclic group.

In some embodiments, the compound of Formula (II) has the structure of Formula (IIa):

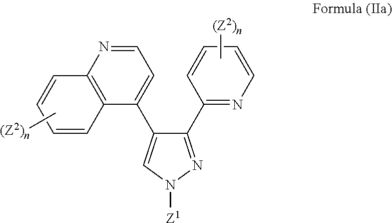

wherein:
Z¹ is H, CONHAr, or CSNHAr;
Z² is a C₁-C₈ straight, branched, or cyclic hydrocarbon group;
Ar is

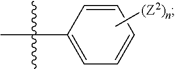

and
each n is independently 0, 1, 2, or 3.

In some embodiments, the compound of Formula (IIa) has the structure:

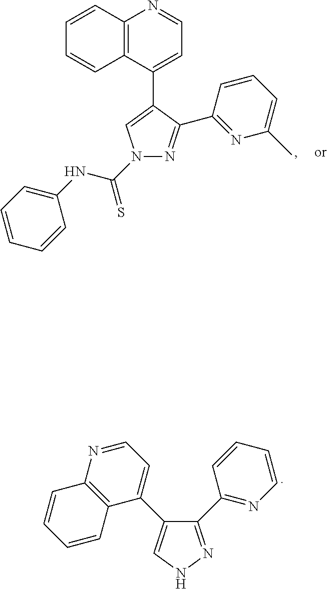

In further embodiments, the compound of Formula (II) has the structure of Formula (IIb):

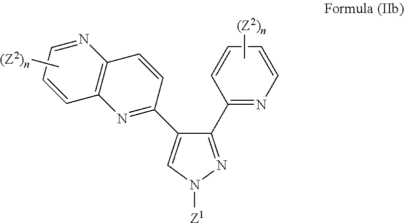

wherein:
Z¹ is H, CONHAr, or CSNHAr;
Z² is a C₁-C₈ straight, branched, or cyclic hydrocarbon group;
Ar is

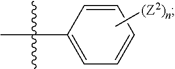

and
each n is independently 0, 1, 2, or 3.

In some cases, the compound of Formula (IIb) has the structure:

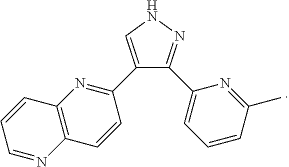

In other embodiments, the TGFβR inhibitor is a compound having the structure of Formula (III):

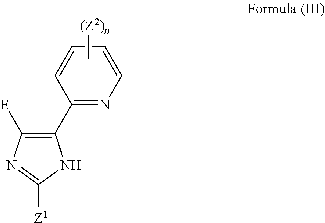

wherein,
E is selected from

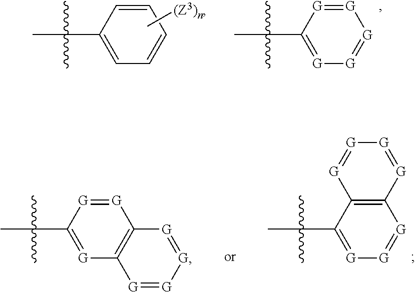

G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is a C₁-C₈ straight, branched, or cyclic hydrocarbon group, or an Ar group;
Ar is

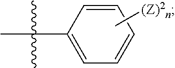

Z² is a C₁-C₈ straight, branched, or cyclic hydrocarbon group, a C₁-C₆ alkoxy group, a —CONH₂ group, or a —CSNH₂ group; and
each n is independently 0, 1, 2, or 3.

In some cases, the compound of Formula (III) has the structure of Formula (IIIa):

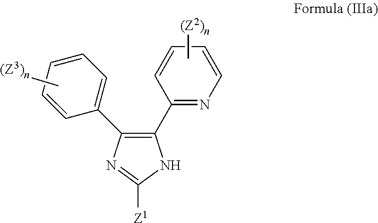

wherein,
Z¹ is C₁-C₈ straight, branched, or cyclic hydrocarbon group, or an Ar group;
Z² is a C₁-C₈ straight, branched, or cyclic hydrocarbon group, a C₁-C₆ alkoxy group, a —CONH₂ group, or a —CSNH₂ group;
Z³ is a C₁-C₈ straight, branched, or cyclic hydrocarbon group, a C₁-C₆ alkoxy group, or two Z³ groups together form a —OCH₂O— or —OCH₂CH₂O— group;
Ar is

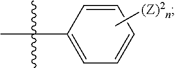

and
each n is independently 0, 1, 2, or 3.

In some embodiments, the compound of Formula (IIIa) has the structure:

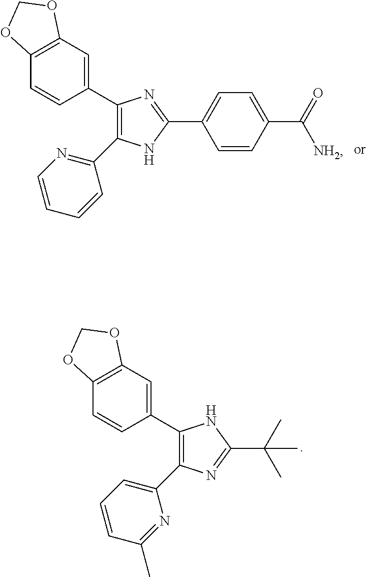

In further embodiments, the TGFβR inhibitor is a compound having the structure of Formula (IV):

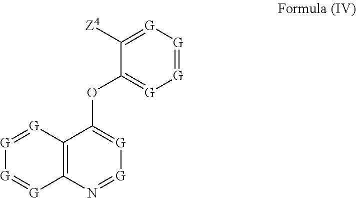

wherein,
G is N, CH or CZ²;
Z² is a C₁-C₈ straight, branched, or cyclic hydrocarbon group, or a C₁-C₆ alkoxy group;
Z⁴ is a COZ² group, a CON(R⁵)₂ group; and
each Z⁵ is independently a hydrogen or C₁-C₈ straight, branched, or cyclic hydrocarbon group.

In some cases, the compound of Formula (IV) has the structure of Formula (IVa):

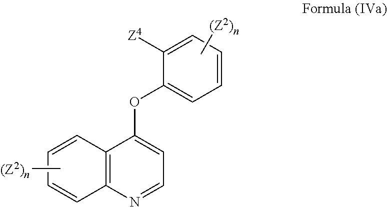

wherein,
Z² is a C₁-C₈ straight, branched, or cyclic hydrocarbon group, a C₁-C₆ alkoxy group;
Z⁴ is a COCH₃ group, a CONH₂ group, a CONH(CH₃) group; and
each n is independently 0, 1, 2, or 3.

In some embodiments, the compound of Formula (IVa) has the structure:

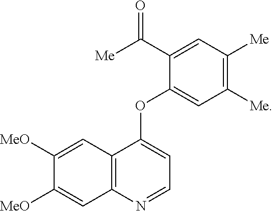

In some embodiments of the above-mentioned method, the enhanced expression is a greater than 2-fold increase in RNA expression compared to cells that have not been contacted with the chemical compound.

In some embodiments, the plurality of human cells having increased potency have teratoma-forming ability.

In some cases, the method also includes the step of forcing the expression in the plurality of human cells of one or more of the following induction factors: Oct3/4, Sox2, Klf4, c-Myc, Lin28, or Nanog. In some cases, the one or more induction factors comprise Oct 3/4, Sox2, and Klf4. In other cases, the one or more induction factors comprise Oct 3/4, Sox2, Klf4, and c-Myc.

In some cases, any of the above-mentioned embodiments, also includes contacting the plurality of human cells with one or more of the following agents: DNA demethylating agent, histone methyltransferase inhibitor, histone deacetylase (HDAC) inhibitor, L-type calcium channel agonist, Wnt ligand, siRNA against p53, siRNA against Utf1 cDNA.

In some embodiments the plurality of human cells to be contacted comprise fibroblasts, blood cells, keratinocytes, hair follicle cells or epithelial cells.

In some cases, the plurality of human cells are derived from a patient. In some embodiments, the plurality of human cells are derived from a patient that is suffering from a neurodegenerative disease or disorder (e.g., Alzheimer's Disease, Parkinson's Disease, Huntington's Disease, or Spinal Muscular Atrophy).

In other embodiments, the plurality of human cells is derived from a patient suffering from a metabolic disorder (e.g., diabetes, obesity, or insulin insensitivity). In yet other embodiments, the plurality of human cells is derived from a patient suffering from hepatic injury.

In another aspect provided herein is a method for generating human induced pluripotent stem cells comprising contacting primary cells obtained from a human subject with a small molecule TGFβ receptor (TGFβR) inhibitor compound and forcing expression of one or more induction factors in the primary cells from the human subject to obtain the human induced pluripotent stem cells.

In some cases, the small molecule TGFβR inhibitor compound is a compound having the structure of Formula (II):

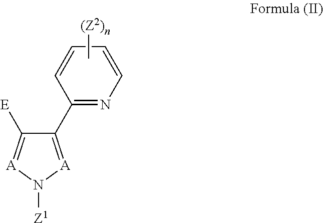

wherein,
E is selected from

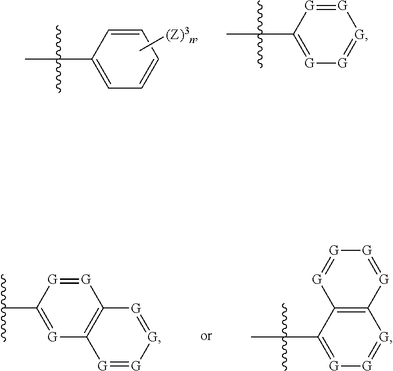

G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is H, CONHAr, or CSNHAr;
Ar is

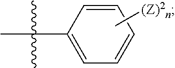

n is 0, 1, 2 or 3; and
each Z² is independently a C₁-C₈ straight, branched or cyclic hydrocarbon group, a C₁-C₆ alkoxy group, or, optionally, two Z² groups, together with the carbon atoms to which said Z² groups are attached, combine to form a cyclic group.

In other embodiments, the small molecule TGFβR inhibitor compound is a compound having the structure of Formula (III):

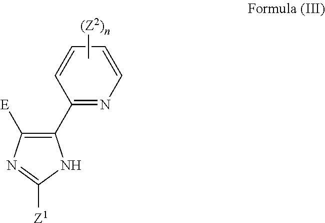

wherein,
E is selected from

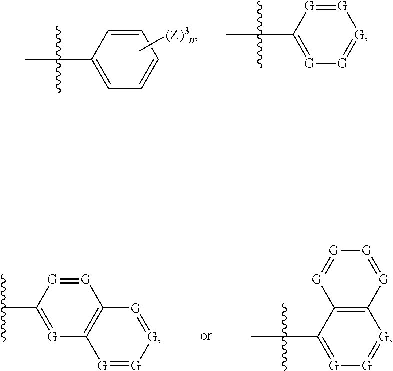

G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is a C₁-C₈ straight, branched, or cyclic hydrocarbon group, or an Ar group;
Ar is

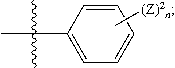

Z² is a C₁-C₈ straight, branched, or cyclic hydrocarbon group, a C₁-C₆ alkoxy group, a —CONH₂ group, or a —CSNH₂ group; and
each n is independently 0, 1, 2, or 3.

In some cases, the small molecule TGFβR inhibitor compound to be used in the above-mentioned method is a compound having the structure of Formula (IV):

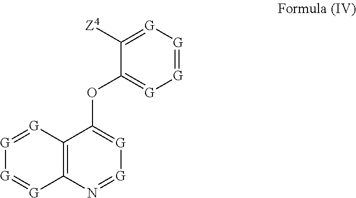

wherein,
G is N, CH or CZ²;
Z² is a C₁-C₈ straight, branched, or cyclic hydrocarbon group, or a C₁-C₆ alkoxy group;
Z⁴ is a COZ² group, a CON(R⁵)₂ group; and
each Z⁵ is independently a hydrogen or C₁-C₈ straight, branched, or cyclic hydrocarbon group.

In some embodiments, a chemical compound is used to increase the expression of an IF in a human differentiated cell (e.g., a blood cell, a dermal cell, a dermal fibroblast, an epithelial cell, a keratinocyte, a hair follicle, etc.). In some aspects, a chemical compound is used to increase the expression of Oct3/4 in a human cell. In other aspects a chemical compound is used to increase the expression of Sox2 in a human cell. In yet other aspects, a chemical compound is used to increase the expression of Klf4 or c-Myc in a human cell. In some embodiments, the chemical compound is contacted with the cell in conjunction with one or more other agents, chemical compounds, nucleic acids, viral vectors, etc. In some embodiments, at some point in time after the contacting of the chemical with the cell, the cell is tested or analyzed for one or more of the following: expression of Oct3/4, expression of Sox2 or expression of Klf4.

In one aspect the invention provides a method of inducing gene expression comprising contacting a plurality of human cells with a compound and identifying at least one pluripotent stem cell in the population, and wherein the compound is:

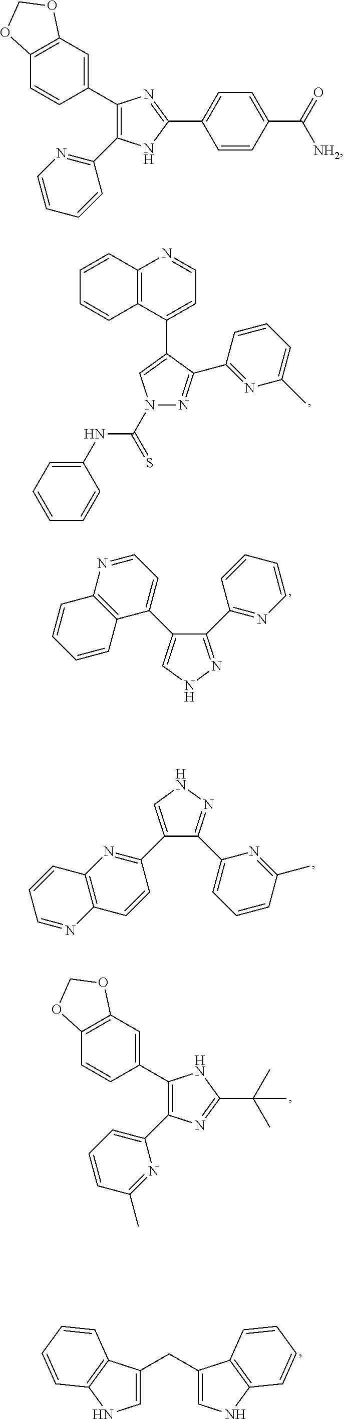

or a compound of Formula I

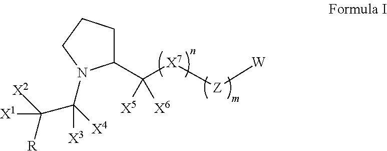

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

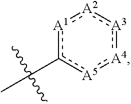

wherein:
   A¹, A², A³, A⁴, and A⁵ are independently N, NR, CR, or CRR; and
each [symbol] is independently a single or double bond, or
   a pharmaceutically acceptable salt of any of the foregoing.

In another aspect, the disclosure provides a method of inducing a human cell to become pluripotent comprising identifying one or more markers of pluripotency in a human cell that has been previously contacted with a chemical compound and wherein the chemical compound is:

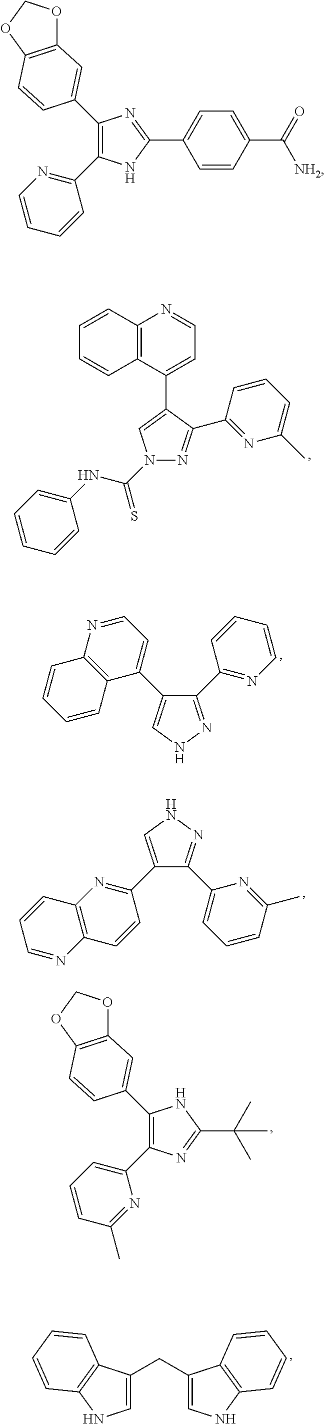

or a compound of Formula I

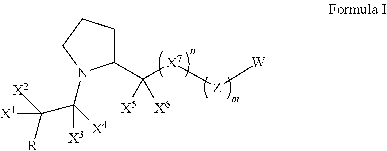

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

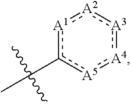

wherein:
   A¹, A², A³, A⁴, and A⁵ are independently N, NR, CR, or CRR; and
   each [symbol] is independently a single or double bond, or
      a pharmaceutically acceptable salt of any of the foregoing.

In another embodiment, the disclosure provides a method of preparing a pluripotent cell comprising contacting a plurality of human cells with:
a. a cytokine, a growth factor, Fibroblast Growth Factor (FGF), bFGF, FGF2, Epidermal Growth Factor (EGF), platelet-derived growth factor (PDGF), or insulin growth factor (IGF); and
b. a chemical compound wherein the chemical compound is:

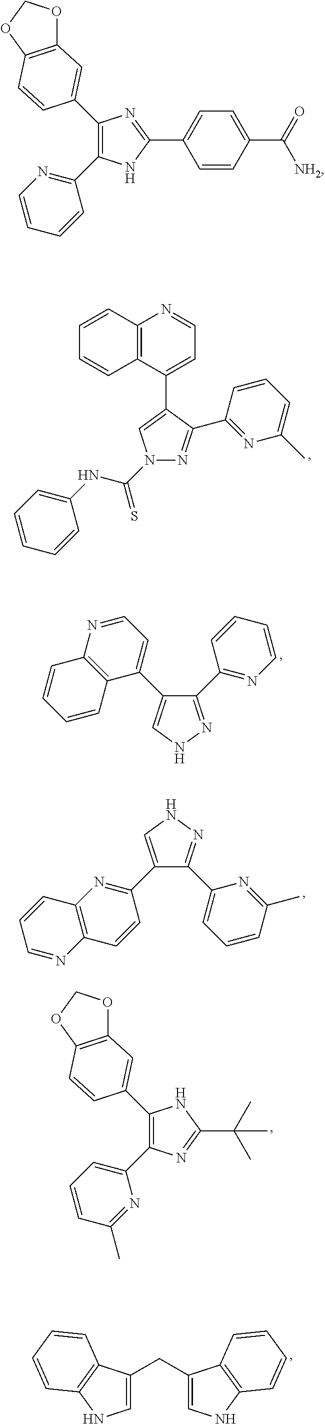

or a compound of Formula I

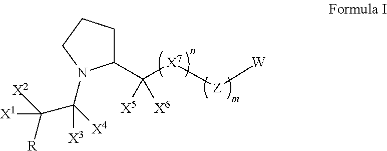

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

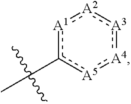

wherein:
   A¹, A², A³, A⁴, and A⁵ are independently N, NR, CR, or CRR; and
   each ═ is independently a single or double bond, or
      a pharmaceutically acceptable salt of any of the foregoing.

In another aspect, the disclosure provides a method of inducing gene expression comprising contacting a cell with a compound with the effect of increasing the expression of Oct3/4, Sox2, Klf4, and/or c-Myc, or comprising measuring the expression of Oct3/4, Sox2, Klf4, and/or c-Myc, and wherein the compound is:

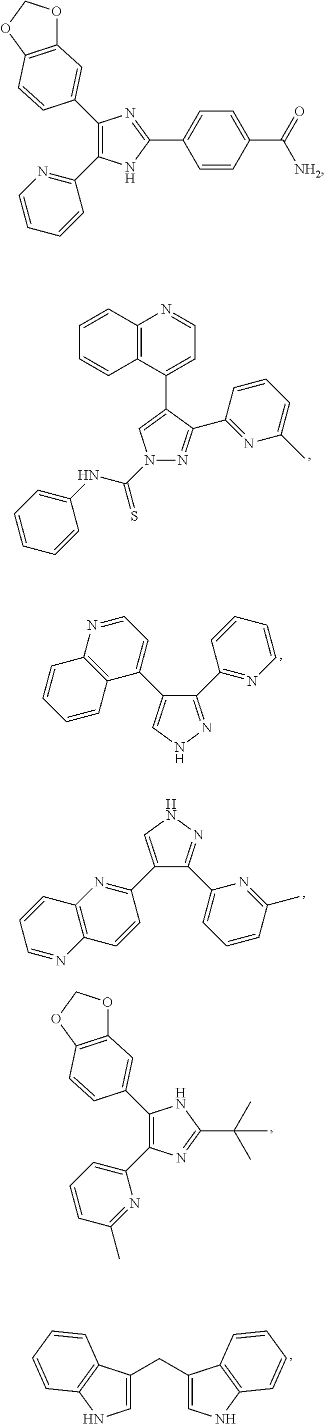

or a compound of Formula I

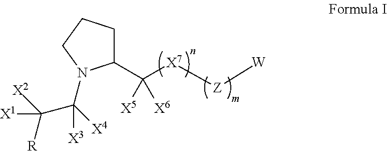

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

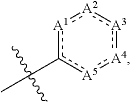

wherein:
   A¹, A², A³, A⁴, and A⁵ are independently N, NR, CR, or CRR; and
   each [symbol] is independently a single or double bond, or
      a pharmaceutically acceptable salt of any of the foregoing.

In another aspect, the disclosure provides a method of increasing the potency of a cell, comprising contacting a cell with a compound and evaluating or analyzing the potency of the contacted cell, or measuring the expression of Oct3/4, Sox2 or Klf4 within the cell, and wherein the compound is:

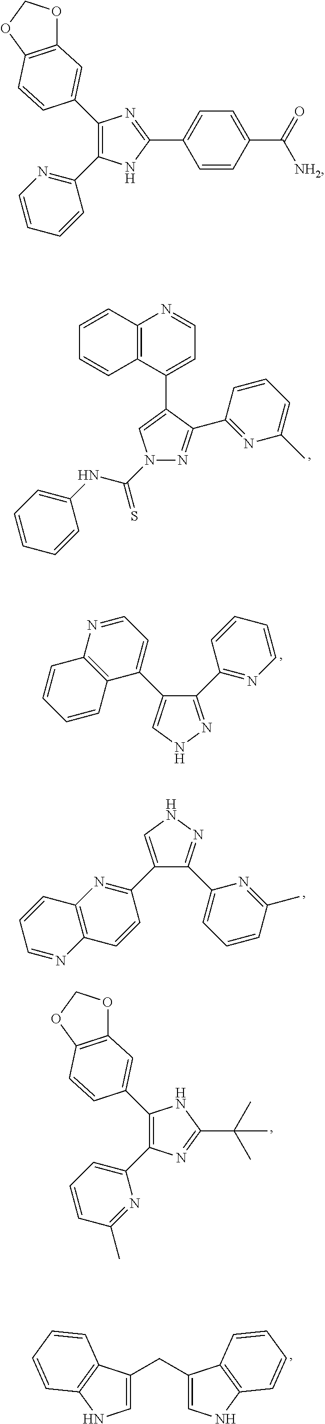

or a compound of Formula I

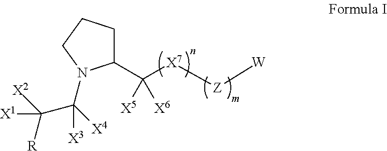

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

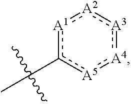

wherein:
   A¹, A², A³, A⁴, and A⁵ are independently N, NR, CR, or CRR; and
   each [symbol] is independently a single or double bond, or
      a pharmaceutically acceptable salt of any of the foregoing.

In another aspect, the disclosure provides a method of enhancing the level of one or more induction factors in a cell comprising, contacting a cell with at least two compounds wherein the compounds are:
(1)

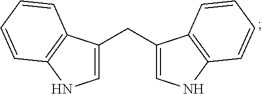

and
(2) a compound of Formula I

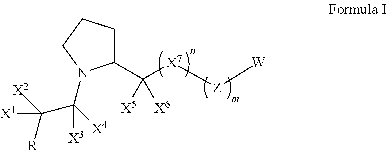

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

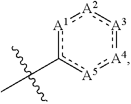

wherein:
   A¹, A², A³, A⁴, and A⁵ are independently N, NR, CR, or CRR; and
   each [symbol] is independently a single or double bond, or
      a pharmaceutically acceptable salt of any of the foregoing.

In another aspect, the disclosure provides a method of inducing gene expression comprising contacting a cell with a compound with the effect of increasing the expression of Oct3/4, Sox2, Klf4, and/or c-Myc, or comprising measuring the expression of Oct3/4, Sox2, Klf4, and/or c-Myc, and wherein the compound is

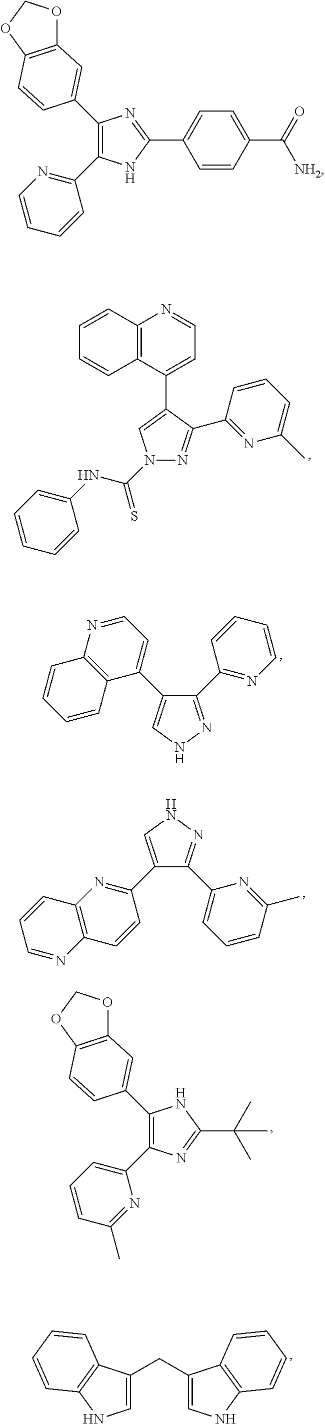

or a compound of Formula I

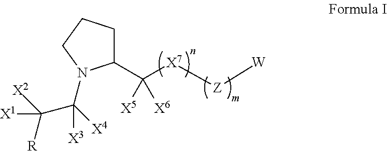

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

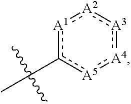

wherein:
   A¹, A², A³, A⁴, and A⁵ are independently N, NR, CR, or CRR; and
   each [symbol] is independently a single or double bond, or
      a pharmaceutically acceptable salt of any of the foregoing.

In another aspect, the disclosure provides a method of enhancing the level of one or more induction factors in a cell comprising, contacting a cell with at least two compounds wherein the compounds are:
(1) a compound and evaluating or analyzing the potency of the contacted cell, or measuring the expression of Oct3/4, Sox2 or Klf4 within the cell, and wherein the compound is:

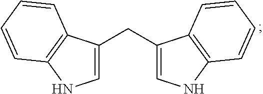

and
(2) a compound of Formula I

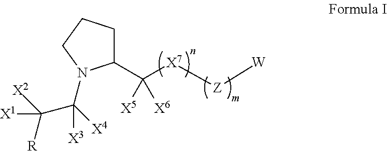

wherein:
X¹ and X² are independently R, OR, or halogen, or together form a carbonyl or a thiocarbonyl;
X³ and X⁴ are independently R, or together form a carbonyl or a thiocarbonyl;
X⁵ and X⁶ are independently R, or together form a carbonyl or a thiocarbonyl;
X⁷ is O, NR, or S;
R is H or a C₁-C₈ straight, branched, or cyclic hydrocarbon group;
Z is a C₁-C₈ straight, branched, or cyclic hydrocarbon group, optionally substituted with R, OR, NRR, or SR;

n is 0 or 1;

m is 0 or 1; and
W is

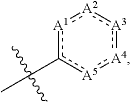

wherein:
   A¹, A², A³, A⁴, and A⁵ are independently N, NR, CR, or CRR; and
   each [symbol] is independently a single or double bond, or
      a pharmaceutically acceptable salt of any of the foregoing.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the effect of GPI-1046 on the relative expression of Sox2 and Oct4 in neonatal human dermal fibroblasts.

FIG. 2 depicts the effect of DIM on the relative expression of p21 and Klf4 in neonatal human dermal fibroblasts.

FIG. 3 depicts the effect of 616 453 on the relative expression of Sox2 in neonatal human dermal fibroblasts.

FIG. 4 depicts the effect of GPI-1046 on the relative expression of Oct4 and Sox2 in neonatal human dermal fibroblasts.

FIG. 5 is an image of neonatal human dermal fibroblasts following treatment with the indicated compound.

FIG. 6 depicts the effect of the indicated TGFβetaR inhibitor on the relative expression of Sox2 in neonatal human dermal fibroblasts.

FIG. 7 depicts the effect of GPI-1046 on the relative expression of Oct4 and Sox2 in neonatal human dermal fibroblasts Day 5 and Day 8 after treatment.

FIG. 8 is an image of neonatal human dermal fibroblasts following infection with viruses encoding Klf4, Sox2, Oct3/4, and c-Myc (“KSOM”). The right panel shows Alkaline Phosphatase (AP) staining of the infected cells

FIG. 9 depicts a dose-response curve of the effect of TGFβR inhibitor compound 616453 on alkaline phosphatase activity induced by the reprogramming factor combination Klf4, Sox2, and Oct3/4 (KSO) in human BJ-5Tα fibroblasts. Alkaline phosphatase activity is detected in a quantitative luminescent substrate assay and quantified by luminometry.

FIG. 10 depicts a dose-response curve of the effect of TGFβR inhibitor compound 616453 on alkaline phosphatase activity induced by the reprogramming factor combination Klf4, Sox2, Oct3/4, and c-Myc (KSOM) in human BJ-5Tα fibroblasts. Alkaline phosphatase activity is detected in a quantitative luminescent substrate assay and quantified by luminometry.

FIG. 11 depicts a dose-response curve of the effect of TGFβR inhibitor compound 616452 on alkaline phosphatase activity induced by the reprogramming factor combination Klf4, Sox2, and Oct3/4 (KSO) in human BJ-5Tα fibroblasts. Alkaline phosphatase activity is detected in a quantitative luminescent substrate assay and quantified by luminometry.

FIG. 12 depicts a dose-response curve of the effect of TGFβR inhibitor compound 616452 on alkaline phosphatase activity induced by the reprogramming factor combination Klf4, Sox2, and Oct3/4 (KSO) in human BJ fibroblasts. Alkaline phosphatase activity is detected in a quantitative luminescent substrate assay and quantified by luminometry.

FIG. 13 depicts a dose-response curve of the effect of TGFβR inhibitor compound 616452 on alkaline phosphatase activity induced by the reprogramming factor combination Klf4, Sox2, Oct3/4, and c-Myc (KSOM) in human BJ fibroblasts. Alkaline phosphatase activity is detected in a quantitative luminescent substrate assay and quantified by luminometry.

FIG. 14 depicts a dose-response curve of the effect of TGFβR inhibitor compound 616452 on alkaline phosphatase activity induced by the reprogramming factor combination Klf4, Sox2, Oct3/4, and c-Myc (KSOM) in human BJ-5Tα fibroblasts. Alkaline phosphatase activity is detected in a quantitative luminescent substrate assay and quantified by luminometry.

FIG. 15 depicts a dose-response curve of the TGFβR inhibitor compound 616452 on the number of alkaline phosphatase positive colonies induced by the reprogramming factor combination Klf4, Sox2, and Oct3/4 (KSO) in human BJ-5Tα fibroblasts. Values are shown as mean (n=4)±SEM; p values were determined by Student's t-test.

FIG. 16 depicts a dose-response curve of the TGFβR inhibitor compound 616452 on the number of alkaline phosphatase positive colonies induced by the reprogramming factor combination Klf4, Sox2, Oct3/4, and c-Myc (KSOM) in human BJ-5Tα fibroblasts. Values are shown as mean (n=4)±SEM; p values were determined by Student's t-test.

FIG. 17 depicts a dose-response curve of the effect of TGFβR inhibitor compound LY3649 on alkaline phosphatase activity induced by the reprogramming factor combination Klf4, Sox2, and Oct3/4 (KSO) in human BJ-5Tα fibroblasts. Alkaline phosphatase activity is detected in a quantitative luminescent substrate assay and quantified by luminometry.

FIG. 18 depicts a dose-response curve of the effect of TGFβR inhibitor compound LY3649 on alkaline phosphatase activity induced by the reprogramming factor combination Klf4, Sox2, Oct3/4, and c-Myc (KSOM) in human BJ-5Tα fibroblasts. Alkaline phosphatase activity is detected in a quantitative luminescent substrate assay and quantified by luminometry.

FIG. 19 depicts a dose-response curve of the effect of TGFβR inhibitor compound SB431542 on alkaline phosphatase activity induced by the reprogramming factor combination Klf4, Sox2, and Oct3/4, (KSO) in human BJ-5Tα fibroblasts. Alkaline phosphatase activity is detected in a quantitative luminescent substrate assay and quantified by luminometry.

FIG. 20 depicts a dose-response curve of the effect of TGFβR inhibitor compound SB431542 on alkaline phosphatase activity induced by the reprogramming factor combination Klf4, Sox2, Oct3/4, and c-Myc (KSOM) in human BJ-5Tα fibroblasts. Alkaline phosphatase activity is detected in a quantitative luminescent substrate assay and quantified by luminometry.

FIG. 21 depicts a dose-response curve of the effect of TGFβR inhibitor compound A83-01 on alkaline phosphatase activity induced by the reprogramming factor combination Klf4, Sox2, and Oct3/4, (KSO) in human BJ-5Tα fibroblasts. Alkaline phosphatase activity is detected in a quantitative luminescent substrate assay and quantified by luminometry.

FIG. 22 depicts a dose-response curve of the effect of TGFβR inhibitor compound A83-01 on alkaline phosphatase activity induced by the reprogramming factor combination Klf4, Sox2, Oct3/4, and c-Myc (KSOM) in human BJ-5Tα fibroblasts. Alkaline phosphatase activity is detected in a quantitative luminescent substrate assay and quantified by luminometry.

FIG. 23 depicts a dose-response curve of the effect of TGFβR inhibitor compound 61645 on alkaline phosphatase activity induced by the reprogramming factor combination Klf4, Sox2, and Oct3/4, (KSO) in human BJ-5Tα fibroblasts. Alkaline phosphatase activity is detected in a quantitative luminescent substrate assay and quantified by luminometry.

FIG. 24 depicts a dose-response curve of the effect of TGFβR inhibitor compound 61645 on alkaline phosphatase activity induced by the reprogramming factor combination Klf4, Sox2, Oct3/4, and c-Myc (KSOM) in human BJ-5Tα fibroblasts. Alkaline phosphatase activity is detected in a quantitative luminescent substrate assay and quantified by luminometry.

FIG. 25 illustrates the chemical structures of exemplary, non-limiting, TGFβR inhibitor compounds that enhance reprogramming of human cells (e.g., fibroblasts) by three reprogramming factor (e.g., KSO) or four reprogramming factor (e.g., KSOM) combinations.

### DETAILED DESCRIPTION OF THE INVENTION

This disclosure provides methods of generating induced pluripotent stem cells or induced multipotent stem cells, which may be useful for drug discovery, diagnostic, and therapeutic applications. The disclosure also provides methods of contacting cells with a particular compound in order to increase the level of, or force the expression of, a gene associated with pluripotency, e.g., Oct3/4, Sox2, Klf4 or c-Myc. Induced pluripotent stem cell lines are distinct from induced multipotent stem cell lines in that the former can be differentiated into cell lineages of all three germ layers, i.e., ectoderm, mesoderm, and endoderm, whereas the latter can be differentiated into a more limited range of cell lineages. For convenience, induced multipotent and pluripotent stem cells are collectively referred to as “induced stem cells” (iSC) below.

### I. Overview

In a typical method described herein, the starting cell is a partially- or fully-differentiated cell (e.g., a blood cell, a dermal cell, a dermal fibroblast, an epithelial cell, a keratinocyte, a hair follicle cell, etc.), which is then contacted with a chemical compound (e.g., small molecule, organic compound, inorganic compound or drug) described herein. Often, the action of the compound causes increased expression, forced expression, or increase in the level of, or enhanced stability of one or more pluripotency-associated genes, or induction factors (“IFs”), e.g., Oct3/4, Sox2, Klf4, c-Myc, Lin28, or Nanog. These factors can be described by various names (e.g., IFs, “induction factors”, pluripotency-associated factors, etc.). In some cases, a chemical compound is used to increase the expression of Oct3/4 and/or Sox2 in a mammalian cell (e.g., human cell). In other cases, a chemical compound is used to increase the expression of Klf4 and/or c-Myc in a human cell. In some cases, one chemical compound is used to enhance the expression of Oct3/4 and/or Sox2 and a second chemical compound is used to enhance expression of Klf4. In some cases, one chemical compound is used to enhance the expression of Oct3/4 and/or Sox2, and a DNA vector or viral vector is used to enhance expression of Klf4 and/or c-Myc. In other cases, one chemical compound is used to enhance the expression of Klf4, and a DNA vector or viral vector is used to enhance expression of Oct3/4, Sox2 and/or c-Myc. Enhanced expression of one or more IFs in a differentiated cell (or lineage-committed cell) may cause the cell to undergo reprogramming (e.g., undergo an increase in potency). Such reprogramming may provide the cell with the potential to differentiate into one or more different cell types. The iSCs disclosed herein have the developmental potential to differentiate into a wide variety of cell types (e.g., neurons, cardiomyocytes, hepatocytes, etc.). iSCs may also possess other features such as the ability to undergo long-term self renewal, expression of certain marker genes identified with pluripotency, the ability to be differentiated in vitro into a variety of cell types and the potential to form a teratoma when injected into a test animal.

In some cases, the compound may be used in conjunction with another mode to force expression of an IF. For example, a compound may be applied to a cell to increase the expression of Oct3/4 and/or Sox2 and a viral or nucleic acid vector may be used to increase the expression of another IF, such as Klf4 or c-Myc.

### II. Compounds of the Invention

Definition of standard chemistry terms may be found in reference works, including Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification.

An “alkoxy” group refers to a (alkyl)O— group, where alkyl is as defined herein.

An “alkyl” group refers to an aliphatic hydrocarbon group. The term “alkyl group” and “hydrocarbon group” are equivalent and may be used interchangeably. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, may be branched, straight chain, or cyclic. Depending on the structure, an alkyl group can be a monoradical or a diradical (i.e., an alkylene group).

As used herein, C₁-Cₓ includes C₁-C₂, C₁-C₃ . . . C₁-Cₓ.

The “alkyl” moiety may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C₁-C₄ alkyl includes C₁-C₂ alkyl and C₁-C₃ alkyl. Alkyl groups can be substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. In some embodiments, the alkyl group is a C₁-C₈ alkyl group. In other embodiments, the alkyl group is a C₁-C₆ alkyl group. In other embodiments, the alkyl group is a C₁-C₄ alkyl group. In other embodiments, the alkyl group is a C₁-C₃ alkyl group. In other embodiments, the alkyl group is a C₁-C₂ alkyl group. In other embodiments, the alkyl group is a C₁ alkyl group.

As used herein, the term “non-cyclic alkyl” refers to an alkyl that is not cyclic (i.e., a straight or branched chain containing at least one carbon atom). Non-cyclic alkyls can be fully saturated or can contain non-cyclic alkenes and/or alkynes. Non-cyclic alkyls can be optionally substituted.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

The term “alkenyl” refers to a type of alkyl group in which the first two atoms of the alkyl group form a double bond that is not part of an aromatic group. That is, an alkenyl group begins with the atoms —C(R)═C(R)—R, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl moiety may be branched, straight chain, or cyclic (in which case, it would also be known as a “cycloalkenyl” group), Depending on the structure, an alkenyl group can be a monoradical or a diradical (i.e., an alkenylene group). Alkenyl groups can be optionally substituted. In some embodiments, the alkenyl group is a C₂-C₈ alkenyl group. In other embodiments, the alkenyl group is a C₂-C₆ alkenyl group. In other embodiments, the alkenyl group is a C₂-C₄ alkenyl group. In other embodiments, the alkenyl group is a C₂-C₃ alkenyl group. In other embodiments, the alkenyl group is a C₂ alkenyl group.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CH₃ and —C≡CCH₂CH₃. The “R” portion of the alkynyl moiety may be branched, straight chain, or cyclic. Depending on the structure, an alkynyl group can be a monoradical or a diradical (i.e., an alkynylene group). Alkynyl groups can be optionally substituted. In some embodiments, the alkynyl group is a C₂-C₈ alkynyl group. In other embodiments, the alkynyl group is a C₂-C₆ alkynyl group. In other embodiments, the alkynyl group is a C₂-C₄ alkynyl group. In other embodiments, the alkynyl group is a C₂-C₃ alkynyl group. In other embodiments, the alkynyl group is a C₂ alkynyl group.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

The term “aromatic” refers to a planar ring having a delocalized 7π-electron system containing 4n+2 π electrons, where n is an integer. Aromatic rings can be formed by five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

As used herein, the term “aryl” refers to an aromatic ring wherein each of the atoms forming the ring is a carbon atom. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

An “aryloxy” group refers to an (aryl)O— group, where aryl is as defined herein.

The term “bond” or “single bond” refers to a chemical bond between two atoms, or two moieties when the atoms joined by the bond are considered to be part of larger substructure.

The term “carbocyclic” refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon.

The term “cycloalkyl” refers to a monocyclic or polycyclic radical that contains only carbon and hydrogen, and may be saturated, partially unsaturated, or fully unsaturated. Cycloalkyl groups include groups having from 3 to 10 ring atoms. Examples of monocyclic cycloalkyls include, e.g., cyclopropyl, cyclobutyl, cyclopentyl, cyclohexyl, cycloheptyl, and cyclooctyl. An unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo-[2.2.1]heptanyl, and the like. Depending on the structure, an cycloalkyl group can be a monoradical or a diradical (e.g., an cycloalkylene group).

As used herein, the term “carbocycle” refers to a ring, wherein each of the atoms forming the ring is a carbon atom. Carbocylic rings can be formed by three, four, five, six, seven, eight, nine, or more than nine carbon atoms. Carbocycles can be optionally substituted.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

The term “halo” or, alternatively, “halogen” or “halide” means fluoro, chloro, bromo or iodo.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

As used herein, the terms “heteroalkyl” “heteroalkenyl” and “heteroalkynyl” include optionally substituted alkyl, alkenyl and alkynyl radicals in which one or more skeletal chain atoms are selected from an atom other than carbon, e.g., oxygen, nitrogen, sulfur, silicon, phosphorus or combinations thereof.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can form part of a ring system.

As used herein, the term “ring system” refers to two or more rings, wherein two or more of the rings are fused. The term “fused” refers to structures in which two or more rings share one or more bonds.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. The polycyclic heteroaryl group may be fused or non-fused. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Depending on the structure, a heteroaryl group can be a monoradical or a diradical (i.e., a heteroarylene group).

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three, four, five, six, seven, eight, nine, or more than nine atoms. Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. The term heteroalicyclic also includes all ring forms of the carbohydrates, including but not limited to the monosaccharides, the disaccharides and the oligosaccharides.

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of the group does not contain two adjacent O or S atoms. Herein, whenever the number of carbon atoms in a heterocycle is indicated (e.g., C₁-C₆ heterocycle), at least one other atom (the heteroatom) must be present in the ring. Designations such as “C₁-C₆ heterocycle” refer only to the number of carbon atoms in the ring and do not refer to the total number of atoms in the ring. It is understood that the heterocyclic ring can have additional heteroatoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In heterocycles that have two or more heteroatoms, those two or more heteroatoms can be the same or different from one another. Heterocycles can be optionally substituted. Binding to a heterocycle can be at a heteroatom or via a carbon atom. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetralydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one. Depending on the structure, a heterocycle group can be a monoradical or a diradical (i.e., a heterocyclene group).

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

An “isocyanato” group refers to a—NCO group.

An “isothiocyanato” group refers to a—NCS group.

The term “ketoalkyl” group refers to an alkyl group substituted with an oxo group.

The term “heteroketoalkyl” group refers to a heteroalkyl group in which one of the carbon atoms is substituted with an oxo group.

The term “moiety” refers to a specific segment or functional group of a molecule. Chemical moieties are often recognized chemical entities embedded in or appended to a molecule.

The term “polycycloalkyl” refers to an alkyl group that comprises a bicyclic or tricyclic ring hydrocarbon ring structure, including bridged cyclalkyl rings, spiro cycloalkyl rings, or fused cycloalkyl rings. Examples include a norbornyl group, an adamantyl group, a bicyclo[x.y.z]alkyl group (where each of x, y, and z is independently 1, 2, 3, or 4), or a tricylic alkyl group.

A “sulfinyl” group refers to a —S(═O)—R.

A “sulfonyl” group refers to a —S(═O)₂—R.

A “thioalkoxy” group refers to a—S-alkyl group.

As used herein, the term “O-carboxy” refers to a group of formula RC(═O)O—.

As used herein, the term “C-carboxy” refers to a group of formula —C(═O)OR.

As used herein, the term “acetyl” refers to a group of formula —C(═O)CH₃.

As used herein, the term “trihalomethanesulfonyl” refers to a group of formula X₃CS(═O)₂— where X is a halogen.

As used herein, the term “cyano” refers to a group of formula —CN.

As used herein, the term “S-sulfonamido” refers to a group of formula —S(═O)₂NR₂.

As used herein, the term “N-sulfonamido” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “0 thiocarbamyl” refers to a group of formula —OC(═S)NR₂.

As used herein, the term “N thiocarbamyl” refers to a group of formula ROC(═S)NH—.

As used herein, the term “C-amido” refers to a group of formula —C(═O)NR₂.

As used herein, the term “N-amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

The term “optionally substituted” or “substituted” means that the referenced group may be substituted with one or more additional group(s) individually and independently selected from alkyl, cycloalkyl, aryl, heteroaryl, heterocyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, isocyanato, thiocyanato, isothiocyanato, nitro, perhaloalkyl, perfluoroalkyl, silyl, and amino, including mono- and di-substituted amino groups, and the protected derivatives thereof. By way of example an optional substituents may be LₛRₛ, wherein each Lₛ is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(substituted or unsubstituted C₁-C₆ alkyl), or -(substituted or unsubstituted C₂-C₆ alkenyl); and each Rₛ is independently selected from H, (substituted or unsubstituted lower alkyl), (substituted or unsubstituted lower cycloalkyl), heteroaryl, or heteroalkyl. The protecting groups that may form the protective derivatives of the above substituents are known to those of skill in the art and may be found in references such as Greene and Wuts, above.

The compounds presented herein may possess one or more stereocenters and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns. The methods and formulations described herein include the use of N-oxides, crystalline forms (also known as polymorphs), or pharmaceutically acceptable salts of compounds described herein, as well as active metabolites of these compounds having the same type of activity. A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

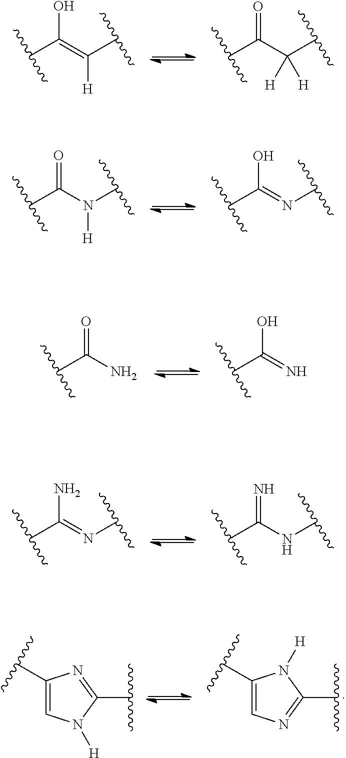

In addition, the compounds described herein can exist in unsolvated as well as solvated forms with pharmaceutically acceptable solvents such as water, ethanol, and the like. The solvated forms of the compounds presented herein are also considered to be disclosed herein.

Throughout the specification, groups and substituents thereof can be chosen by one skilled in the field to provide stable moieties and compounds.

The compounds of the Invention include three categories of compounds: Category A, Category B, and Category C.

Category A compounds include compounds of Formula I, or a pharmaceutically acceptable salt thereof:

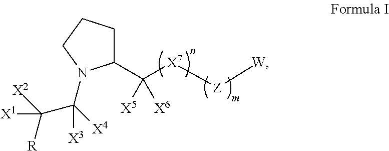

wherein:

X¹ and X² are independently R, OR, or halogen, or together form a carbonyl or a thiocarbonyl;

X³ and X⁴ are independently R, or together form a carbonyl or a thiocarbonyl;

X⁵ and X⁶ are independently R, or together form a carbonyl or a thiocarbonyl;

X⁷ is O, NR, or S;

R is H or a C₁-C₈ straight, branched, or cyclic hydrocarbon group;

Z is a C₁-C₈ straight, branched, or cyclic hydrocarbon group, optionally substituted with R, OR, NRR, or SR;

n is 0 or 1;

m is 0 or 1; and

W is

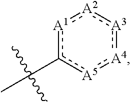

wherein:
A¹, A², A³, A⁴, and A⁵ are independently N, NR, CR, or CRR; and
each [symbol] is independently a single or double bond, or
   a pharmaceutically acceptable salt of any of the foregoing.

Category A compounds also include a compound of Formula I or a pharmaceutically acceptable salt thereof, wherein:

X¹ and X² are independently R, OR, or halogen, or together form a carbonyl or a thiocarbonyl;

X³ and X⁴ together form a carbonyl;

X⁵ and X⁶ together form a carbonyl;

X⁷ is O;

R is H or a C₁-C₈ straight, branched, or cyclic hydrocarbon group;

Z is a C₁-C₈ straight, branched, or cyclic hydrocarbon group, optionally substituted with R, OR, NRR, or SR;

n is 1;

m is 0 or 1; and

W is

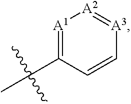

wherein:
A¹, A², and A³ are independently N or CR.

Category A compounds also include a compound of Formula I, or a pharmaceutically acceptable salt thereof, wherein:

X¹ and X² together form a carbonyl;

X³ and X⁴ together form a carbonyl;

X⁵ and X⁶ together form a carbonyl;

X⁷ is O;

R is H or a C₁-C₈ straight, branched, or cyclic hydrocarbon group;

Z is —(CH₂)—(CH₂)—(CH₂)—;

n is 1;

m is 1; and

W is

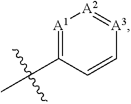

wherein:

one of A¹, A², and A³ is N, and the others are CR.

Category A compounds also include a compound of Formula I, wherein the compound is

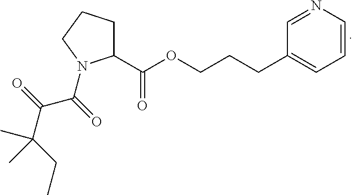

Category A compounds also include a compound of Formula I, wherein the compound is

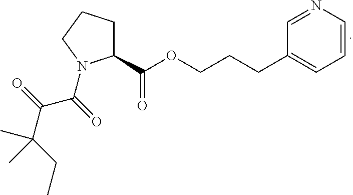

Category A compounds may also include: one or more proline derivatives, or pharmaceutically acceptable salt thereof; one or more non-immunosuppressive immunophilin ligands, or pharmaceutically acceptable salt thereof, or the compound known as GPI-1046, or pharmaceutically acceptable salt thereof.

Category B compounds include TGF-β (TGFβ) inhibitors or TGF-β receptor (TGFβR) inhibitors, or pharmaceutically acceptable salt thereof. In some cases, a TGFβR inhibitor is a compound having the structure of Formula (II):

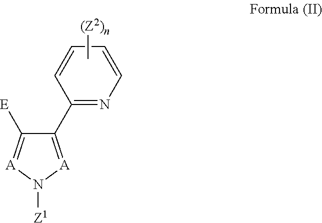

wherein,

E is selected from

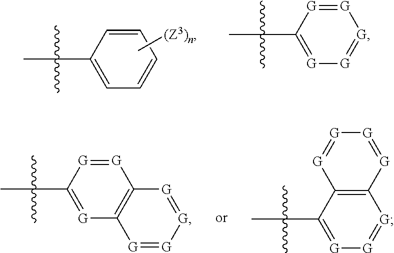

G is N, O, CH or CZ³, wherein at least one G is N;

each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;

Z¹ is H, CONHAr, or CSNHAr;

Ar is

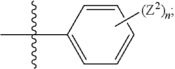

n is 0, 1, 2 or 3; and

each Z² is independently a C₁-C₈ straight, branched or cyclic hydrocarbon group, a C₁-C₆ alkoxy group, or, optionally, two Z² groups, together with the carbon atoms to which said Z² groups are attached, combine to form a cyclic group.

In some cases the TGFβ inhibitor compound having the structure of Formula (II) has the structure of Formula (IIa):

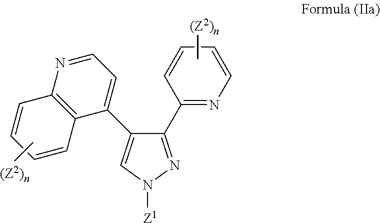

wherein:

Z¹ is H, CONHAr, or CSNHAr;

Z² is a C₁-C₈ straight, branched, or cyclic hydrocarbon group;

Ar is

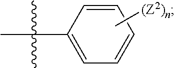

and

each n is independently 0, 1, 2, or 3.

Non-limiting examples of compounds of Formula (IIa) include, but are not limited to:

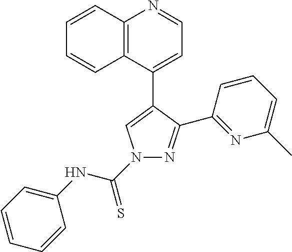

referred to herein as “A83-01” (or “A83”); or the compound

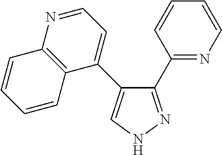

referred to herein as “LY3649” (or “LY”).

In other embodiments, a TGFβR inhibitor compound having the structure of Formula (II) has the structure of Formula (IIb):

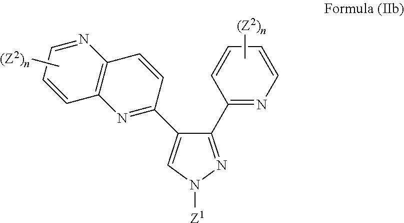

wherein:

Z¹ is H, CONHAr, or CSNHAr;

Z² is a C₁-C₈ straight, branched, or cyclic hydrocarbon group;

Ar is

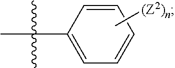

and

each n is independently 0, 1, 2, or 3.

In one non-limiting embodiment, the structure of the TGFβR inhibitor compound having the structure of Formula (IIb) has the structure:

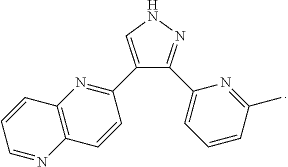

The above TGFβR inhibitor compound is also referred to herein as “616452”.

In some cases, a TGFβR inhibitor is a compound having the structure of Formula (III):

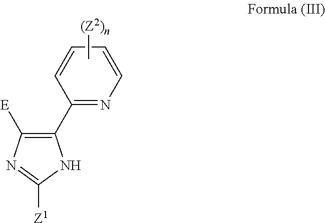

wherein,

E is selected from

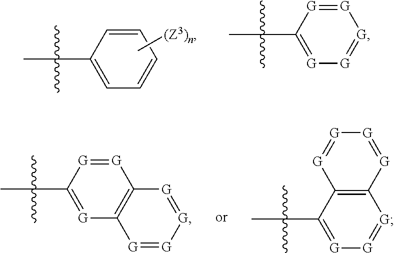

G is N, O, CH or CZ³, wherein at least one G is N;

each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;

Z¹ is a C₁-C₈ straight, branched, or cyclic hydrocarbon group, or an Ar group;

Ar is

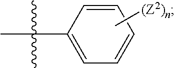

Z² is a C₁-C₈ straight, branched, or cyclic hydrocarbon group, a C₁-C₆ alkoxy group, a —CONH₂ group, or a —CSNH₂ group; and

each n is independently 0, 1, 2, or 3.

In some embodiments, a TGFβR inhibitor compound having the structure of Formula (III) has the structure of Formula (IIIa):

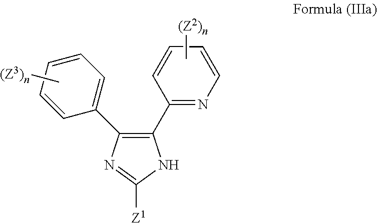

wherein,

Z¹ is C₁-C₈ straight, branched, or cyclic hydrocarbon group, or an Ar group;

Z² is a C₁-C₈ straight, branched, or cyclic hydrocarbon group, a C₁-C₆ alkoxy group, a —CONH₂ group, or a —CSNH₂ group;

Z³ is a C₁-C₈ straight, branched, or cyclic hydrocarbon group, a C₁-C₆ alkoxy group, or two Z³ groups together form a —OCH₂O— or —OCH₂CH₂O— group;

Ar is

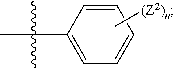

and

each n is independently 0, 1, 2, or 3.

In some non-limiting embodiments, the structure of the TGFβR inhibitor compound having the structure of Formula (IIIa) has the structure:

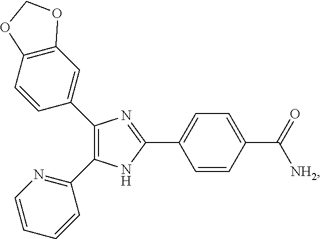

referred to herein as “SB431542” (or “SB”); or the compound:

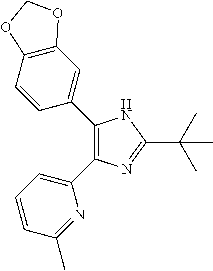

referred to herein as “616453”.

In further embodiments, a TGFβR inhibitor compound is compound having the structure of Formula (IV):

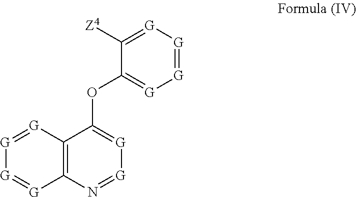

wherein,

G is N, CH or CZ²;

Z² is a C₁-C₈ straight, branched, or cyclic hydrocarbon group, or a C₁-C₆ alkoxy group;

Z⁴ is a COZ² group, a CON(R⁵)₂ group; and

each Z⁵ is independently a hydrogen or C₁-C₈ straight, branched, or cyclic hydrocarbon group.

In some embodiments, the TGFβR inhibitor compound of Formula (IV) is a compound having the structure of Formula (IVa):

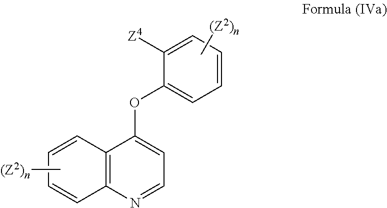

wherein,

Z² is a C₁-C₈ straight, branched, or cyclic hydrocarbon group, a C₁-C₆ alkoxy group;

Z⁴ is a COCH₃ group, a CONH₂ group, a CONH(CH₃) group; and

each n is independently 0, 1, 2, or 3.

In some embodiments the TGFβR compound having the structure of Formula (IVa) has the structure:

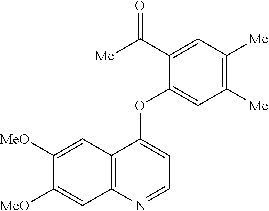

referred to herein as “616458.”
III. Increasing the Level of Induction factor(s)

In some cases, the disclosure provides a method of inducing gene expression comprising identifying one or more markers of pluripotency in a human cell (e.g., alkaline phosphatase) that has been previously contacted with a chemical compound and wherein the chemical compound is a compound of the invention, including Category A, B, or C compounds.

In another aspect, the disclosure provides a method of increasing the potency of a human cell comprising contacting a plurality of human cells (e.g., human primary cells) with a Category A, B, or C compound and forcing expression of one or more induction factors to obtain a plurality of human cells having increased potency. Such induction factors include, but are not limited to one or more of, Oct3/4, Klf4, Sox2, c-Myc, Nanog, and Lin-28. In some embodiments, the induction factors include Oct 3/4, Klf4, Sox2. In other embodiments, the induction factors include Oct 3/4, Klf4, Sox2, and c-Myc. In some cases, the Category B compound to be used in the just-mentioned method is a TGFβR inhibitor compound, e.g., a TGFβR inhibitor compound of Formula (II), Formula (III), or Formula (IV).

In one aspect, the disclosure provides a method of generating a human pluripotent cell (e.g., a human induced pluripotent stem cell) comprising contacting a plurality of human primary cells with a compound of the invention including Category A, B, or C compounds and forcing the expression of one or more induction factors. Such induction factors include, but are not limited to one or more of, Oct3/4, Klf4, Sox2, c-Myc, Nanog, and Lin-28. In some embodiments, the induction factors include Oct 3/4, Klf4, Sox2. In other embodiments, the induction factors include Oct 3/4, Klf4, Sox2, and c-Myc. In some cases, the Category B compound to be used in the just-mentioned method is a TGFβR inhibitor compound, e.g., a TGFβR inhibitor compound of Formula (II), Formula (III), or Formula (IV).

In some cases, the disclosure provides a method of inducing gene expression comprising contacting one or more cells, or a plurality of human cells, with a chemical compound and identifying one or more markers of pluripotency or multipotency expressed by the cell, wherein the chemical compound is a compound of the invention including Category A, B, or C compounds.

In some cases, the disclosure provides a method of increasing the level of one or more induction factors in a cell, comprising contacting one or more cells, or a plurality of human cells, with a compound of the invention, e.g., Category A, B, and/or C compounds.

In some cases, the disclosure provides a method of increasing the level of one or more induction factors in a cell, comprising contacting one or more cells, or a plurality of human cells, with

(1) a first chemical compound, wherein the first chemical compound is a Category A compound or a pharmaceutically acceptable salt thereof: and

(2) a second chemical compound, wherein the second chemical compound is a Category C compound or a pharmaceutically acceptable salt thereof.

In some cases, the disclosure provides a method of increasing the level of a induction factor in a cell, comprising contacting one or more cells, or a plurality of human cells, with

(1) a first chemical compound, wherein the first chemical compound is a compound of Category A; and

(2) a second chemical compound, wherein the second chemical compound is a compound of Category B.

In some cases, the disclosure provides a method of increasing the level of a induction factor in a cell, comprising contacting one or more cells, or a plurality of human cells, with

(1) a first chemical compound, wherein the first chemical compound is a compound of Category B; and

(2) a second chemical compound, wherein the second chemical compound is a compound of Category C.

In some cases, the disclosure provides a method of increasing the level of a induction factor in a cell, comprising contacting one or more cells, or a plurality of human cells, with

(1) a first chemical compound, wherein the first chemical compound is a compound of Category A, B; or C, and

(2) a second chemical compound, wherein the second chemical compound is a compound of HDAC inhibitor.

In some cases, the disclosure provides a composition or pharmaceutical composition, comprising:

(1) a first chemical compound, wherein the first chemical compound is a Category A compound or a pharmaceutically acceptable salt thereof: and

(2) a second chemical compound, wherein the second chemical compound is a Category B compound or a pharmaceutically acceptable salt thereof.

In some cases, the disclosure provides a composition or pharmaceutical composition, comprising:

(1) a first chemical compound, wherein the first chemical compound is a Category A compound or a pharmaceutically acceptable salt thereof: and

(2) a second chemical compound, wherein the second chemical compound is a Category C compound or a pharmaceutically acceptable salt thereof.

In some cases, the disclosure provides a composition or pharmaceutical composition, comprising:

(1) a first chemical compound, wherein the first chemical compound is a Category B compound or a pharmaceutically acceptable salt thereof: and

(2) a second chemical compound, wherein the second chemical compound is a Category C compound or a pharmaceutically acceptable salt thereof.

In some cases, the disclosure provides a composition or pharmaceutical composition, comprising:

(1) a first chemical compound, wherein the first chemical compound is a Category A, B, or C compound or a pharmaceutically acceptable salt thereof: and

(2) a second chemical compound, wherein the second chemical compound is a HDAC inhibitor compound or a pharmaceutically acceptable salt thereof.

The concentration of Category A compound (e.g., GPI-1046), Category B compound (e.g., 616452), or Category C compound may be any effective concentration, including, but not limited to: greater than 0 μM, greater than about 1 μM, greater than about 5 μM, greater than about 10 μM, greater than about 20 μM, greater than about 30 μM, greater than about 50 μM, greater than about 100 μM G; less than about 1 μM, less than about 5 μM, less than about 10 μM, less than about 20 μM, less than about 30 μM, less than about 50 μM, less than about 100 μM, about 1 μM, about 5 μM, about 10 μM, about 20 μM, about 30 μM, about 50 μM, or about 100 μM.

In some cases, the disclosure provides a method of increasing the level of one or more induction factors in a cell, comprising contacting one or more cells, or a plurality of human cells, with a Category A compound (e.g., a proline derivative such as GPI-1046, or pharmaceutically acceptable salt thereof), and a Category C compound (e.g., a DIM compound). In some embodiments, the concentration of Category A compounds (e.g., GPI-1046) is about 1 μM, about 5 μM, about 10 μM, about 20 μM, about 30 μM, about 50 μM, or about 100 μM; and the concentration of Category C compound (e.g., a DIM compound) is about 1 μM, about 5 μM, about 10 μM, about 20 μM, about 30 μM, about 50 μM, or about 100 μM.

Pharmaceutically acceptable salts include, but are not limited to, hydrochloride, hydrobromide, hydroiodide, oxalate, carbonate, bicarbonate, nitrate, sulfate, sulfite, bisulfate, phosphate, acid phosphate, isonicotinate, lactate, salicylate, tartrate, bitartrate, ascorbate, gentisinate, gluconate, glucaronate, saccarate, formate, benzoate, glutamate, pantothenate, acetate, fumarate, succinate, methanesulfonate, ethanesulfonate, benzenesulfonate, p-toluoylsulfonate, citrate, or maleate salts.

At some point after contacting the cells with a compound of the invention (e.g., a Category A, B, or C compound), the cells may have about a 1.3, 1.5, 2, 2.5, 3, 3.5, 4, 5, or 6-fold increase or greater than 1, 2, 3, 4, 5, or 6-fold increase in Oct3/4, Sox2, Klf4, and/or c-Myc expression. For example, contacting one or more cells with a Category A compound (e.g., Formula I compound, GPI-1046, etc.) may result in a greater than 1.5-, 2-, 3-, 4-, 5-, or 6-fold increase in Sox2 (e.g., Sox2 mRNA or Sox2 protein) expression.

In some cases, the method comprises contacting a cell with a compound described herein and then, after about 1, 2, 3, 4, 5, 6, 7, 8, 9, 10, 11, 12, 13, 14, 15, 16, 17, 18, 19, 20, 25, 30, 35, 40, 45, 50, 55, or 60 days, measuring the expression of one or more of the following genes: Oct3/4, Sox2, Klf4, c-Myc, SSEA-3, SSEA-4, TRA-1-60, TRA-1-81, TRA-2-49/6E (alkaline phosphatase), Nanog, GDF3, REX1, FGF4, ESG1, DPPA2, DPPA4, and hTERT.

In some cases, the method also comprises analyzing a cell for pluripotency or multipotency. The analysis may include identifying markers of pluripotency such as the appearance of colonies or colonies with an iPS-cell-like or Embryonic-Stem-cell like morphology. In some cases, wherein the marker of pluripotency is enhanced expression of one or more of the genes in the group consisting of: SSEA-3, SSEA-4, TRA-1-60, TRA-1-81, TRA-2-49/6E (alkaline phosphatase), Nanog, Oct-3/4, Sox2, GDF3, REX1, FGF4, ESG1, DPPA2, DPPA4, and hTERT. The analysis may also be an analysis of the teratoma-forming potential of the cell, as described herein or of the long-term self renewal capabilities of the cell, as described herein.

In some cases, the method comprises contacting one or more cells with one or more compounds of the invention (e.g., Category A, B, or C compounds) and also contacting the one or more cells with one or more of the following agents: DNA demethylating agent, histone methyltransferase inhibitor, histone deacetylase (HDAC) inhibitor, L-type calcium channel agonist, Wnt ligand, siRNA against p53, siRNA against Utf1 cDNA, a transducible protein, TGFβ inhibitor, TGFβR inhibitor, DIM, or non-immunosuppressive immunophilin ligand. Some examples in the literature of such additional factors that could be added include, but are not limited to: histone deacetylase (HDAC) inhibitors, see, e.g., Huangfu et al. (2008) Nature Biotechnol. 26:795-797; Huangfu et al. (2008) Nature Biotechnol. 26: 1269-1275; DNA demethylating agents, see, e.g., Mikkelson et al. (2008) Nature 454, 49-55; histone methyltransferase inhibitors, see, e.g., Shi et al. (2008) Cell Stem Cell 2:525-528; L-type calcium channel agonists, see, e.g., Shi et al. (2008) Cell Stem Cell 3:568-574; Wnt3a, see, e.g., Marson et al. (2008) Cell 134:521-533; and siRNA, see, e.g., Zhao et al. (2008) Cell Stem Cell 3: 475-479. These methods are described further herein. In some examples, one or more cells are contacted with a proline derivative, a chemical compound of Formula I, a non-immunosuppressive immunophilin ligand, or GPI-1046; and with one or more HDAC inhibitors e.g., valproic acid, as described herein. See, e.g., Huangfu et al. (2008) Nature Biotechnol. 26: 1269-1275.

In some cases, the method comprises contacting one or more cells with one or more compounds of the invention (e.g., Category A, B, or C compounds) and also contacting the one or more cells with a viral vector or nucleic acid (e.g., DNA, RNA) vector encoding one or more induction factors. For example, one or more cells may be contacted with (A) a Category A compound, a proline derivative, a compound of Formula I described above, or GPI-1046; and with (B) a viral or nucleic acid vector encoding (1) Klf4 alone; (2) Klf4 and Sox2; (3) Klf4 and c-Myc; (4) Klf4 and Oct4; (5) Sox2, Klf4, and c-Myc; (6) Oct4, Klf4 and c-Myc. In preferred embodiments, one or more cells are contacted with GPI-1046 (or a pharmaceutically-acceptable salt thereof) and with a viral or nucleic acid vector encoding Klf4. In some examples, one or more cells are contacted with (1) a Category A compound, a proline derivative, a chemical compound of Formula I, a non-immunosuppressive immunophilin ligand, or GPI-1046; (2) an HDAC inhibitor, e.g., valproic acid, see, e.g., Huangfu et al. (2008) Nature Biotechnol. 26:795-797; and (3) a viral or nucleic acid vector encoding Klf4 and/or c-Myc.

In some cases, one or more cells are contacted with (A) a Category B compound, e.g., a compound of Formula (II), Formula (III), or Formula (IV) and with (B) a viral or nucleic acid vector encoding (1) Oct3/4; (2) Oct3/4 and Klf4; (3) Klf4; (4) Klf4 and c-Myc; or (5) Oct3/4, Klf4, and c-Myc; (6) Klf4, Sox2, and Oct3/4; (7) Klf4, Sox2, and Oct3/4, and c-Myc; or encoding any other combination of the induction factors Oct3/4, Sox2, Klf4, c-Myc, Nanog, and Lin-28.

In some cases, one or more cells are contacted with (A) a Category C compound and with (B) a viral or nucleic acid vector encoding (1) Oct3/4 or Sox2; (2) Oct3/4 and Sox2; or (3) Oct3/4, Sox2, and c-Myc.

Methods of introducing viral or nucleic acid vectors into a cell are described herein.

Methods of generating human iPS cells are known in the art, and a wide range of methods can be used to generate iPS cells or iSC and may be combined with contacting one or more cells with one or more compounds of the invention (e.g., Category A, B, or C compounds). See, e.g., Takahashi and Yamanaka (2006) Cell 126:663-676; Yamanaka et al. (2007) Nature 448:313-7; Wernig et al. (2007) Nature 448:318-24; Maherali (2007) Cell Stem Cell 1:55-70; Maherali and Hochedlinger (2008) Cell Stem Cell 3:595-605; Park et al. (2008) Cell 134:1-10; Dimos et. al. (2008) Science 321:1218-1221; Blelloch et al. (2007) Cell Stem Cell 1:245-247; Stadtfeld et al. (2008) Science 322:945-949; Stadtfeld et al. (2008) Cell Stem Cell 2:230-240; Okita et al. (2008) Science 322:949-953; Kaji et al. (2009) Virus-free induction of pluripotency and subsequent excision of induction factors, Nature advance online publication, 1 Mar. 2009|doi:10.1038/nature07864; Woltjen et al. (2009) piggyBac transposition reprograms fibroblasts to induced pluripotent stem cells, Nature advance online publication, 1 Mar. 2009|doi:10.1038/nature07863. Methods for inducing multipotent and pluripotent stem cell lines are further disclosed in U.S. Patent publication number 20090191159.

A. Histone Deacetylase Inhibitors (HDAC)

The disclosure includes contacting cells with one or more compounds of the invention (e.g., Category A, B, or C compounds) and with one or more HDAC inhibitors.

The HDAC inhibitor treatment of the cells may be combined with one or more compounds of the invention shown to increase the expression of Oct3/4 and/or Sox2 (e.g., a Category A compound, GPI-1046, a compound of Formula I, a non-immunosuppressive immunophilin ligand, any pharmaceutically-acceptable salt thereof, etc.).

Cells may be treated with one or more HDACs for about 2 hours to about 5 days, e.g., 3 hours, 6 hours, 12 hours, 14 hours, 18 hours, 1 day, 2 days, 3 days, or 4 days. Treatment with HDAC inhibitor may be initiated prior to addition of one or more of the chemical compounds described herein. In some cases, HDAC inhibitor treatment begins during or after addition of one or more of the chemical compounds described herein. In other cases, HDAC inhibitor treatment begins prior to f addition of one or more of the chemical compounds described herein and is maintained during addition of one or more of the chemical compounds described herein.

Suitable concentrations of an HDAC inhibitor range from about 0.001 nM to about 10 mM, depending on the particular HDAC inhibitor to be used, but are selected so as to not significantly decrease cell survival in the treated cells. The HDAC inhibitor concentration may range from 0.01 nM, to 1000 nM. In some embodiments, the HDAC concentration ranges from about 0.01 nM to about 1000 nM, e.g., about 0.05 nM, 0.1 nM, 0.5 nM, 0.75 nM, 1.0 nM, 1.5 nM, 10 nM, 20 nM, 40 nM, 50 nM, 100 nM, 200 nM, 300 nM, 500 nM, 600 nM, 700 nM, 800 nM, or other concentration from about 0.01 nM to about 1000 nM. In some cases, the HDAC inhibitor concentration is greater than 1000 nM, greater than 10 μM, greater than 100 μM, greater than 200 μM, greater than 500 μM, or greater than 1 mM. Cells are exposed for about 1 to 3 days, 1 to 5 days, or greater than 5 days. For example, cells are exposed 1 day, 2 days, 3 days, 4 days or 5 days.

Multiple varieties of HDAC inhibitors can be used for the induction experiments. In a preferred embodiment, the HDAC inhibitor MS-275 is used. Examples of suitable HDAC inhibitors include, but are not limited to, any the following:

A. Trichostatin A and its analogs, for example: trichostatin A (TSA); and trichostatin C (Koghe et al. 1998, Biochem. Pharmacol. 56: 1359-1364).

B. Peptides, for example: oxamflatin [(2E)-5-[3-[(phenylsulfonyl)aminophenyl]-pent-2-ene-4-inohydroxamic acid (Kim et al., Oncogene 18: 2461-2470 (1999)); Trapoxin A (cylco-(L-phenylalanyl-L-phenylalanyl-D-pipecolinyl-L-2-amino-8-oxo-9,10-epoxy-decanoyl) (Kijima et al., J. Biol. Chem. 268: 22429-22435 (1993)); FR901228, depsipeptide (Nakajima et al., Ex. Cell RES. 241: 126-133 (1998)); FR225497, cyclic tetrapeptide (H. Mori et al., PCT International Patent Publication WO 00/08048 (Feb. 17, 2000)); apicidin, cyclic tetrapeptide [cyclo-(N—O-methyl-L-tryptophanyl-L-isoleucinyl-D-pipecolinyl-L-2-amino-8-oxodecanoyl)] (Darkin-Rattray et al., Proc. Natl. Acad. Sci. U.S.A. 93: 13143-13147 (1996); apicidin Ia, apicidin Ib, apicidin Ic, apicidin IIa, and apicidin IIb (P. Dulski et al., PCT International Patent Publication WO 97/11366); HC-toxin, cyclic tetrapeptide (Bosch et al., Plant Cell 7: 1941-1950 (1995)); WF27082, cyclic tetrapeptide (PCT International Patent Publication WO 98/48825); and chlamydocin (Bosch et al., supra).

C. Hybrid polar compounds (HPC) based on hydroxamic acid, for example: salicyl hydroxamic acid (SBHA) (Andrews et al., International J. Parasitology 30: 761-8 (2000)); suberoylanilide hydroxamic acid (SAHA) (Richon et al., Proc. Natl. Acad. Sci. U.S.A. 95: 3003-7 (1998)); azelaic bishydroxamic acid (ABHA) (Andrews et al., supra); azelaic-1-hydroxamate-9-anilide (AAHA) (Qiu et al., Mol. Biol. Cell 11: 2069-83 (2000)); M-carboxy cinnamic acid bishydroxamide (CBHA) (Ricon et al., supra); 6-(3-chlorophenylureido) carpoic hydroxamic acid, 3-Cl-UCHA) (Richon et al., supra); MW2796 (Andrews et al., supra); and MW2996 (Andrews et al., supra).

D. Short chain fatty acid (SCFA) compounds, for example: sodium butyrate (Cousens et al., J. Biol. Chem. 254: 1716-23 (1979)); isovalerate (McBain et al., Biochem. Pharm. 53: 1357-68 (1997)); valproic acid; valerate (McBain et al., supra); 4-phenyl butyric acid (4-PBA) (Lea and Tulsyan, Anticancer Research 15: 879-3 (1995)); phenyl butyric acid (PB) (Wang et al., Cancer Research 59: 2766-99 (1999)); propinate (McBain et al., supra); butylamide (Lea and Tulsyan, supra); isobutylamide (Lea and Tulsyan, supra); phenyl acetate (Lea and Tulsyan, supra); 3-bromopropionate (Lea and Tulsyan, supra); tributyrin (Guan et al., Cancer Research 60: 749-55 (2000)); arginine butyrate; isobutyl amide; and valproate.

E. Benzamide derivatives, for example: MS-275 [N-(2-aminophenyl)-4-[N-(pyridine-3-yl-methoxycarbonyl)aminomethyl]benzamide] (Saito et al., Proc. Natl. Acad. Sci. U.S.A. 96: 4592-7 (1999)); and a 3′-amino derivative of MS-275 (Saito et al., supra); and CI-994.

A histone deacetylase inhibitor treatment may be carried out, for example, as follows. The concentration of the HDAC inhibitor may depend on a particular inhibitor, but is preferably 0.001 nM to about 10 mM, or 0.01 nM to about 1000 nM. The effective amount or the dosage of a histone deacetylase inhibitor is defined as the amount of the histone deacetylase inhibitor that does not significantly decrease the survival rate of cells. Cells are exposed for 1 to 2 says, 1-5 days or 1 to 10 days. The exposure period may be less than one day. In a specific embodiment, cells are cultured for about 1 to 5 days, and then exposed to an effective amount of a histone deacetylase inhibitor. However, the histone deacetylase inhibitor may be added at the start of culturing. Within such a time frame, cells may be contacted with one or more compounds described herein (e.g., GPI-1046, a compound of Formula I, a non-immunosuppressive immunophilin ligand, any pharmaceutically-acceptable salt thereof, etc.) and/or a viral or nucleic acid vector encoding Oct3/4, Sox2, Klf4, and/or c-Myc.

B. DNA Demethylating Agents

Induction of the cells may be accomplished according to some embodiments of the present methods by combining treatment of cells with one or more compounds of the invention (e.g., Category A, B, or C compounds) with one or more DNA demethylating agents. Methylation contributing to epigenetic inheritance can occur through DNA methylation. DNA methylation in vertebrates typically occurs at CpG (cytosine-phosphate-guanine) sites, which methylation results in the conversion of the cytosine to 5-methylcytosine.

The DNA methyltransferase (DNMT) family of enzymes catalyze the transfer of a methyl group to DNA. The formation of Me-CpG is catalyzed by the DNA methyltransferases such as, i.e., DNMT1, 2 and 3. CpG sites are uncommon in vertebrate genomes but are often found at higher density near vertebrate gene promoters where they are collectively referred to as CpG islands. The methylation state of CpG sites can have a major impact on gene activity/expression. Demethylating agents are compounds that can inhibit methylation of DNA sequences, resulting in the expression of the previously hypermethylated silenced genes. Exemplary DNA demethylating agents include, without limitation, cytidine analogs such as 5-azacytidine (azacitidine) and 5-azadeoxycytidine (decitabine). These compounds work by binding to DNA methyltransferases, the enzymes that catalyze the methylation reaction, which binding titrates out these enzymes to reduce or eliminate activity (Holliday and Ho (2002) Methods 27 (2): 179-83). Both compounds have been approved in the treatment of myelodysplastic syndrome (MDS) by Food and Drug Administration (FDA) in the United States. Azacitidine and decitabine are marketed as Vidaza® and Dacogen® respectively. Azacitidine is approved by the FDA for treating MDS (Issa et al. (2005) Nat Rev Drug Discov 4 (4): 275-6; Gore et al. (2006). “Decitabine” Nat Rev Drug Discov 5 (11): 891-2.)

In some embodiments of the present methods, one or more cells are contacted with one or more compounds of the invention (e.g., Category A, B, or C compounds) and a DNMT. Since hypomethylation is known to induce apoptosis in differentiated cells, whereas embryonic stem cells are resistant (Jackson-Grusby et al. (2001) Nature Genet. 27, 31-39; Lei et al. (1996) Development 122, 3195-3205; Meissner et al. (2005) Nucleic Acids Res. 33, 5868-5877), the methyltransferase inhibitors can be added a sufficient period of time after commencing induction of dedifferentiation or reprogramming such that cytotoxicity is minimized and the frequency of dedifferentiation is maximized. The skilled artisan can readily determine such a time period by performing a time course in which methyltransferase inhibitor is added at various points following induction factor exposure, as is known in the art. An amount of methyltransferase inhibitor effective in improving the efficiency of dedifferentiation can be added, such as about 0.1 μM, generally about 0.5 μM, sometimes 1 μM, or more as needed for the cell type being treated (see, e.g., Mikkelsen, et al. (2008) Nature 454, 49-55).

Alternatively, targeted methods of inhibiting the expression of the methyltransferase inhibitor itself can be employed, such as, for example, by inhibition and/or degradation of the RNA encoding it. An example of modulation of gene expression by target RNA degradation is RNA interference (RNAi). RNAi is a form of antisense-mediated gene silencing involving the introduction of dsRNA leading to the sequence-specific reduction of targeted endogenous mRNA levels. Short interfering RNA (siRNA) molecules of use to this end include a double stranded RNA that comprises about 19 base pairs of a target gene sequence and is capable of inhibiting target gene expression of RNA interference. See, e.g., Scherr et al., (2007), Cell Cycle, 6(4):444-449.

Antisense technology is an effective means for reducing the expression of one or more specific gene products and can therefore prove to be uniquely useful in a number of therapeutic, diagnostic, and research applications. Chemically modified nucleosides are routinely used for incorporation into antisense compounds to enhance one or more properties, such as nuclease resistance, pharmacokinetics or affinity for a target RNA. Generally, the principle behind antisense technology is that an antisense compound hybridizes to a target nucleic acid and effects modulation of gene expression activity or function, such as transcription, translation or splicing.

The modulation of gene expression can also be achieved by, for example, target degradation or occupancy-based inhibition. An example of modulation of RNA target function by degradation is RNase H-based degradation of the target RNA upon hybridization with a DNA-like antisense compound.

In general, their sequence-specificity makes these and other techniques which target the products of genes expressing DNMTs attractive as tools for enhancing the efficiency of pluripotency induction. The use of any such methods as known in the art are envisioned as being of use to the present methods.

C. Histone Methyltransferase Inhibitors

In some embodiments of the present methods, one or more cells are contacted with one or more compounds of the invention (e.g., Category A, B, or C compounds) and one or more histone methyltransferase inhibitors (e.g., BIX01294). The histone methyltransferase inhibitor BIX01294 has been shown to restore reprogramming efficiency in neural progenitor cells with Oct4/Klf4 to those expressing all of Oct4, Sox2, Klf4 and c-Myc polypeptides (Shi et al. (2008) Cell Stem Cell 2, 525-528).

Histone modifying enzymes including histone methyltransferases have been implicated in the formation of cancer via negative regulation of tumor suppressor genes. U.S. Patent Publication No. 20050266473 teaches a method for identifying compounds that inhibit histone methyltransferases for use in treating cancer, which application is herein incorporated by reference in its entirety. Since histone methyltransferases affect the transcrptional availability of genes, their inhibition positively influences the efficiency of reprogramming in the presence of factors which promote pluripotency. The presence of BIX01294 has been demonstrated to permit the reprogramming of mouse neural progenitor cells in the absence of Oct4 and in the presence of Sox2, Klf4 and c-Myc. It is envisioned that that other histone methyltransferase inhibitors with established activity in the art may find use in the present methods.

D. Calcium Channel Agonists

In some embodiments of the present methods, one or more cells are contacted with one or more compounds of the invention (e.g., Category A, B, or C compounds) and one or more calcium channel agonist, e.g., BayK8644 L-type calcium channel agonist. It has been shown that BayK8644 L-type calcium channel agonist cooperates with BIX01294 to enable reprogramming of mouse embryonic fibroblasts in the presence of Oct4 and Klf4 (Shi et al. (2008) Cell Stem Cell 3, 568-574). This and other calcium channel agonists as known in the art may therefore find use in combination with IFs and other small molecules in promoting induction of pluripotency according to the present methods.

E. Wnt3a Cell Signaling Molecule

In some embodiments of the present methods, one or more cells are contacted with one or more compounds of the invention (e.g., Category A, B, or C compounds) and one or more Wnt cell signaling molecules (wnts). Wnts promote both differentiation of midbrain dopaminergic cells and self-renewal of haematopoietic stem cells. It has been demonstrated that conditioned medium prepared from cells expressing wnt3a can replace feeder cell layers and medium containing LIF in maintaining mouse ES cells in a self-renewing state, while conditioned medium from cells expressing wnt11 cannot (Singla et al. (2006) Biochem Biophys Res Commun. 345(2):789-95). Wnt3a promotes the transcriptional activation of multiple downstream targets, including c-Myc. Incubation of mouse fibroblasts with Wnt3a yields a 1.2-fold enhancement of reprogramming efficiency with Oct4, Sox2, Klf4 and c-Myc (Marson et al. (2008) Cell 134, 521-533) and about 20-fold enhancement in the presence of factors Oct4, Sox2 and Klf4. Accordingly, signaling molecules such as Wnt3a which are known or discovered to positively regulate the expression of genes associated with pluripotency, directly or through intermediary signalling molecules, are of interest to the present methods as factors which can obviate the need for exogenous expression of IFs.

F. Supporting Factors

The presently disclosed methods can further include additional factors which influence or amplify the effects of the IFs previously described without themselves being capable of functional replacement of any of the IFs. Such supporting factors can thus be present or introduced during induction without the removal or replacement of other IFs so as to enhance the efficiency of pluripotency induction. The addition of an upstream inducer or a downstream effector of any of Oct4, Sox2, Klf4 and c-Myc function are of interest to the present methods. By way of example, it has been demonstrated that the expression of UTF1 significantly increases the efficiency of iPS/iSC generation (Zhao et al. (2008) Cell Stem Cell 3, 475-479). UTF1 possesses histone-like properties and is able to function as a stable chromatin-associated transcriptional repressor (van den Boom et al. (2007) J. Cell Biol. 178, 913-924). Without wishing to be bound by theory, UTF1 may promote a histone configuration which favors pluripotency over more differentiated states, through the establishment of an epigenetic profile or a specific chromatin state susceptible to appropriate cell fate stimuli. UTF1 is also reported to be a downstream factor of the OCT4/SOX2 complex, highly expressed in ESCs and downregulated at the onset of differentiation (Nishimoto et al. (1999) Mol. Cell. Biol. 19, 5453-5465). It has been theorized that OCT4 and SOX2 may act through UTF1 in the iPSC reprogramming process. UTF1 is not sufficient to replace other factors, but enhances their ability to induce pluripotency. It is thus envisioned that other factors known or determined to enhance the effects of genes which induce pluripotency can be of use in the present methods.

Modulators of tumor suppressor expression or activity may also be of interest. For example, siRNA directed against the p53 tumor suppressor by itself enhances the efficiency of iPSC generation in the presence of Oct4, Sox2, Klf4 and c-Myc. (Zhao et al. (2008) Cell Stem Cell 3, 475-479). When UTF1 is combined with p53 in the presence of the other four factors, 100-fold enhancement of efficiency of induction occurs, while either factor individually cannot replace any of the four IFs. Other such factors known to the art can likewise be used to supplement IFs so as to increase the efficiency of induction.

G. Viral or Nucleic Acid Vectors

Inducing a cell to become multipotent or pluripotent can be accomplished in numerous ways. In some embodiments, the methods for induction of pluripotency or multipotency in one or more cells include adding one or more compounds of the invention (e.g., Category A, B, or C compounds) and adding a viral or nucleic acid vector capable of forcing expression of a set of induction factors (IFs). In some cases, only a vector(s) encoding Klf4 is introduced to the cells. In some cases, only a vector encoding Klf4 or c-Myc is introduced to the cells. In some cases, only a vector encoding c-Myc is introduced to the cells. In some cases, only a vector encoding Oct3/4 and/or Sox2 is introduced to the cells. In some cases, the set of IFs is one or more: an Oct3/4 polypeptide, a Sox2 polypeptide, a Klf4 polypeptide, or a c-Myc polypeptide. In some cases, the set does not include a c-Myc polypeptide. For example, the set of IFs can include: an Oct3/4 polypeptide, a Sox2 polypeptide, and a Klf4 polypeptide, but not a c-Myc polypeptide. In some cases, the set of IFs does not include polypeptides that might increase the risk of cell transformation.

In some cases, the set may include a c-Myc polypeptide. In certain cases, the c-Myc polypeptide is a constitutively active variant of c-Myc. In some instances, the set includes a c-Myc polypeptide capable of inducible activity, e.g., a c-Myc-ER polypeptide, see, e.g., Littlewood, et al. (1995) Nucleic Acid Res. 23(10):1686-90.

In some cases, the set of IFs includes three IFs, wherein two of the three IFs are an Oct3/4 polypeptide and a Sox2 polypeptide. In other cases, the set of IFs includes two IFs, wherein the two polypeptides are a c-Myc polypeptide and a Sox2 polypeptide; an Oct3/4 polypeptide and a Sox2 polypeptide; a Klf4 polypeptide and a c-Myc polypeptide; a c-Myc polypeptide and an Oct3/4 polypeptide; a Sox2 polypeptide and a Klf4 polypeptide; or an Oct3/4 polypeptide and a Klf4 polypeptide. In some cases, the set of induction factors is limited to Oct 3/4, Sox2, and Klf4 polypeptides. In other cases, the set of induction factors may be limited to a set of four IFs: an Oct3/4 polypeptide, a Sox2 polypeptide, a Klf4 polypeptide, and a c-Myc polypeptide.

A set of IFs may include IFs in addition to an Oct 3/4, a Sox2, and a Klf4 polypeptide. Such additional IFs include, but are not limited to Nanog, TERT, LIN28, CYP26A1, GDF3, FoxD3, Zfp42, Dnmt3b, Ecat1, and Tel1 polypeptides. In some cases, the set of additional IFs does not include a c Myc polypeptide. In some cases, the set of additional IFs does not include polypeptides that might increase the risk of cell transformation.

Forced expression of IFs may be maintained for a period of at least about 7 days to at least about 40 days, e.g., 8 days, 9 days, 10 days, 11 days, 12 days, 13 days, 14 days, 15 days, 16 days, 17 days, 18 days, 19 days, 20 days, 21 days, 25 days, 30 days, 33 days, or 37 days.

The efficiency of inducing pluripotency in cells of a human population of cells is from at least about 0.001% to at least about 0.01% of the total number of cells to be induced, e.g., 0.002%, 0.0034%, 0.004%, 0.005%, 0.0065%, 0.007%, 0.008%, or 0.0085%.

Forced expression of the IFs may comprise introducing one or more mammalian expression vectors encoding an Oct 3/4, a Sox2, and a Klf4 polypeptide to a population of cells. The IFs may be introduced into the cells as exogenous genes. In some cases, the exogenous genes are integrated into the genome of a host cell and its progeny. In other cases, the exogenous genes persist in an episomal state in the host cell and its progeny. Exogenous genes are genes that are introduced to the cell from an external source. A gene as used herein is a nucleic acid that includes an open reading frame encoding a polypeptide of interest, e.g., an IF. The gene preferably includes a promoter operably linked to an open reading frame. In some cases, a natural version of the gene may already exist in the cell but an additional “exogenous gene” is added to the cell to induce polypeptide expression.

The one or more mammalian expression vectors may be introduced into greater than 20% of the total population of cells, e.g., 25%, 30%, 35%, 40%, 44%, 50%, 57%, 62%, 70%, 74%, 75%, 80%, 90%, or other percent of cells greater than 20%. A single mammalian expression vector may contain two or more of the just-mentioned IFs. In other cases, one or more expression vectors encoding an Oct 3/4, Sox2, Klf4, and c Myc polypeptide are used. In some embodiments, each of the IFs to be expressed is encoded on a separate mammalian expression vector.

In some cases, the IFs are genetically fused in frame with a transport protein amino acid sequence, e.g., that of a VP22 polypeptide as described in, e.g., U.S. Pat. Nos. 6,521,455, 6,251,398, and 6,017,735. Such VP22 sequences confer intercellular transport of VP22 fusion polypeptides from cells that have been transfected with a VP22 fusion polypeptide expression vector to neighboring cells that have not been transfected or transduced. See, e.g., Lemken et al. (2007), Mol Ther, 15(2):310-319. Accordingly, the use of IF-VP22 fusion polypeptides can significantly increase the functional efficiency of transfected mammalian expression vectors in the induction methods described herein.

Examples of suitable mammalian expression vectors include, but are not limited to: recombinant viruses, nucleic acid vectors, such as plasmids, bacterial artificial chromosomes, yeast artificial chromosomes, human artificial chromosomes, cDNA, cRNA, and PCR product expression cassettes. Examples of suitable promoters for driving expression of IFs in include retroviral LTR elements; constitutive promoters such as CMV, HSV1-TK, SV40, EF-1, βactin; PGK, and inducible promoters, such as those containing Tet-operator elements. In some cases, one or more of the mammalian expression vectors encodes, in addition to an IF, a marker gene that facilitates identification or selection of cells that have been transfected or infected. Examples of marker genes include, but are not limited to, fluorescent protein genes, e.g., for EGFP, DS-Red, YFP, and CFP; proteins conferring resistance to a selection agent, e.g., the neoR gene, and the blasticidin resistance gene.

1. Recombinant Viruses

Forced expression of an IF may be accomplished by introducing a recombinant virus carrying DNA or RNA encoding an IF to one or more cells. Additionally, the recombinant virus may carry DNA or RNA encoding more than 1 IF. This includes multiple copies of a single IF or multiple IFs containined within a single virus. For ease of reference, at times a virus will be referred to herein by the IF it is encoding. For example, a virus encoding an Oct3/4 polypeptide, may be described as an “Oct3/4 virus.” In certain cases, a virus may encode more than one copy of an IF or may encode more than one IF, e.g., two IFs, at a time.

Different combinations or sets of recombinant viruses may be introduced to the cells. The set of recombinant viruses may include combinations included in any set of IFs described herein. The set of recombinant viruses may include at least: an Oct3/4 virus, a Sox2 virus, and a Klf4 virus. The set of recombinant viruses may be limited to a set of four recombinant viruses: an Oct3/4 virus, a Sox2 virus, a Klf4 virus, and a c-Myc virus. In some cases, the set of recombinant viruses is limited to a set of at least: an Oct3/4 virus, a Sox2 virus, a Klf4 virus, and a c-Myc virus. In some cases, the set of recombinant viruses is limited to Oct 3/4, Sox2, and Klf4 viruses. The set of recombinant viruses may be limited a set of at least: an Oct3/4 virus, a Sox2 virus, and a Klf4 virus. In some cases, the set of recombinant viruses includes three recombinant viruses, wherein two of the three recombinant viruses are an Oct3/4 virus and a Sox2 virus. In still other cases, the set of recombinant viruses may be limited to a Sox2 virus and a c-Myc virus.

In some cases, the set of recombinant viruses does not include a recombinant virus that encodes a polypeptide that might increase the risk of cell transformation, e.g., a c-Myc polypeptide. For example, the set of recombinant viruses can include: an Oct3/4 virus, a Sox2 virus, and a Klf4 virus but not a c-Myc virus.

In other cases, the set of recombinant viruses includes a c-Myc virus. The c-Myc polypeptide encoded by the c-Myc virus may be wild-type c-Myc or a constitutively active variant of c-Myc. In some instances, the set includes a virus encoding c-Myc polypeptide capable of inducible activity, e.g., a c-Myc-ER polypeptide, see, e.g., Littlewood, et al. (1995) Nucleic Acid Res. 23(10):1686-90.

The set of recombinant viruses may include: an Oct3/4 virus, a Sox2 virus, and a Klf4 virus, but not a TERT virus, a SV40 Large T antigen virus, HPV16 E6 virus, a HPV16 E7 virus, or a Bmi1 virus. At times, the set of recombinant viruses does not include a TERT virus. In some cases, the set of recombinant viruses does not include a SV40 virus. In other cases, the set of recombinant viruses does not include a HPV16 E6 virus or a HPV16 E7 virus.

A set of recombinant viruses may include viruses in addition to an Oct 3/4, a Sox2, and a Klf4 virus. Such additional recombinant viruses include, but are not limited to Nanog, TERT, CYP26A1, GDF3, FoxD3, Zfp42, Dnmt3b, Ecat1, and Tel1 viruses. In some cases, thet set of recombinant viruses includes any IF variant described herein.

Individual viruses may be added to the cells sequentially in time or simultaneously. In some cases, at least one virus, e.g., an Oct3/4 virus, a Sox2 virus, a Klf4 virus, or a c-Myc virus, is added to the cells at a time different from the time when one or more other viruses are added. In some examples, the Oct3/4 virus, Sox2 virus and KlF4 virus are added to the cells simultaneously, or very close in time, and the c-Myc virus is added at a time different from the time when the other viruses are added.

At least two recombinant viruses may be added to the cells simultaneously or very close in time. In some examples, Oct3/4 virus and Sox2 virus are added simultaneously, or very close in time, and the Klf4 virus or c-Myc virus is added at a different time. In some examples, Oct3/4 virus and Sox2 virus; Oct3/4 virus and Klf4 virus; Oct3/4 virus and c-Myc virus; Sox2 virus and Klf4 virus; Sox2 virus and c-Myc virus; or Klf4 and c-Myc virus are added simultaneously or very close in time.

In some cases, at least three viruses, e.g., an Oct3/4 virus, a Sox2 virus, and a Klf4 virus, are added to the cells simultaneously or very close in time. In other instances, at least four viruses, e.g., Oct3/4 virus, Sox2 virus, Klf4 virus, and c-Myc virus are added to the cells simultaneously or very close in time.

At times, the efficiency of viral infection can be improved by repetitive treatment with the same virus. In some cases, one or more Oct3/4 virus, Sox2 virus, Klf4 virus, or c-Myc virus is added to the cells at least two, at least three, or at least four separate times.

Examples of recombinant viruses include, but are not limited, to retroviruses (including lentiviruses); adenoviruses; and adeno-associated viruses. Often, the recombinant retrovirus is murine moloney leukemia virus (MMLV), but other recombinant retroviruses may also be used, e.g., Avian Leukosis Virus, Bovine Leukemia Virus, Murine Leukemia Virus (MLV), Mink-Cell focus-Inducing Virus, Murine Sarcoma Virus, Reticuloendotheliosis virus, Gibbon Abe Leukemia Virus, Mason Pfizer Monkey Virus, or Rous Sarcoma Virus, see, e.g., U.S. Pat. No. 6,333,195.

In other cases, the recombinant retrovirus is a lentivirus (e.g., Human Immunodeficiency Virus-1 (HIV-1); Simian Immunodeficiency Virus (SIV); or Feline Immunodeficiency Virus (FIV)), See, e.g., Johnston et al. (1999), Journal of Virology 73(6)″4991-5000 (Fly); Nègre D et al. (2002) Current Topics in Microbiology and Immunology 261:53-74 (SIV); Naldini et al. (1996) Science 272:263-267 (HIV).

The recombinant retrovirus may comprise a viral polypeptide (e.g., retroviral env) to aid entry into the target cell. Such viral polypeptides are well-established in the art, see, e.g., U.S. Pat. No. 5,449,614. The viral polypeptide may be an amphotropic viral polypeptide, e.g., amphotropic env, that aids entry into cells derived from multiple species, including cells outside of the original host species. See, e.g., id. The viral polypeptide may be a xenotropic viral polypeptide that aids entry into cells outside of the original host species. See, e.g., id. In some embodiments, the viral polypeptide is an ecotropic viral polypeptide, e.g., ecotropic env, that aids entry into cells of the original host species. See, e.g., id.

Examples of viral polypeptides capable of aiding entry of retroviruses into cells include but are not limited to: MMLV amphotropic env, MMLV ecotropic env, MMLV xenotropic env, vesicular stomatitis virus-g protein (VSV-g), HIV-1 env, Gibbon Ape Leukemia Virus (GALV) env, RD114, FeLV-C, FeLV-B, MLV 10A1 env gene, and variants thereof, including chimeras. See e.g., Yee et al. (1994), Methods Cell Biol. Pt A:99-112 (VSV-G); U.S. Pat. No. 5,449,614. In some cases, the viral polypeptide is genetically modified to promote expression or enhanced binding to a receptor.

In general, a recombinant virus is produced by introducing a viral DNA or RNA construct into a producer cell. In some cases, the producer cell does not express exogenous genes. In other cases, the producer cell is a “packaging cell” comprising one or more exogenous genes, e.g., genes encoding one or more gag, pol, or env polypeptides and/or one or more retroviral gag, pol, or env polypeptides. The retroviral packaging cell may comprise a gene encoding a viral polypeptide, e.g., VSV-g that aids entry into target cells. In some cases, the packaging cell comprises genes encoding one or more lentiviral proteins, e.g., gag, pol, env, vpr, vpu, vpx, vif, tat, rev, or nef. In some cases, the packaging cell comprises genes encoding adenovirus proteins such as E 1A or E1B or other adenoviral proteins. For example, proteins supplied by packaging cells may be retrovirus-derived proteins such as gag, pol, and env, lentivirus-derived proteins such as gag, pol, env, vpr, vpu, vpx, vif, tat, rev, and nef; and adenovirus-derived proteins such as E1A and E1B. In many examples, the packaging cells supply proteins derived from a virus that differs from the virus from which the viral vector derives.

Packaging cell lines include but are not limited to any easily-transfectable cell line. Packaging cell lines can be based on 293T cells, NIH3T3, COS or HeLa cell lines. As packaging cells, any cells may be used that can supply a lacking protein of a recombinant virus vector plasmid deficient in at least one gene encoding a protein required for virus packaging. Examples of packaging cell lines include but are not limited to: Platinum-E (Plat-E); Platinum-A (Plat-A); BOSC 23 (ATCC CRL 11554); and Bing (ATCC CRL 11270), see, e.g., Morita et al. (2000) Gene Therapy 7:1063-1066; Onishi et al. (1996) Experimental Hematology 24:324-329; U.S. Pat. No. 6,995,009. Commercial packaging lines are also useful, e.g., Ampho-Pak 293 cell line, Eco-Pak 2-293 cell line, RetroPack PT67 cell line, and Retro-X Universal Packaging System (all available from Clontech).

The retroviral construct may be derived from a range of retroviruses, e.g., MMLV, HIV-1, SIV, FIV, or other retrovirus described herein. The retroviral construct may encode all viral polypeptides necessary for more than one cycle of replication of a specific virus. In some cases, the efficiency of viral entry is improved by the addition of other factors or other viral polypeptides. In other cases, the viral polypeptides encoded by the retroviral construct do not support more than one cycle of replication, e.g., U.S. Pat. No. 6,872,528. In such circumstances, the addition of other factors or other viral polypeptides can help facilitate viral entry. In an exemplary embodiment, the recombinant retrovirus is HIV-1 virus comprising a VSV-g polypeptide but not comprising a HIV-1 env polypeptide.

The retroviral construct may comprise: a promoter, a multi-cloning site, and/or a resistance gene. Examples of promoters include but are not limited to CMV, SV40, EF1, β actin; retroviral LTR promoters, and inducible promoters. The retroviral construct may also comprise a packaging signal (e.g., a packaging signal derived from the MFG vector; a psi packaging signal). Examples of retroviral constructs known in the art include but are not limited to: pMX, pBabeX or derivatives thereof. See e.g., Onishi et al. (1996) Experimental Hematology 24:324-329. In some cases, the retroviral construct is a self-inactivating lentiviral vector (SIN) vector, see, e.g., Miyoshi et al., (1998) J. Virol. 72(10): 8150-8157. In some cases, the retroviral construct is LL-CG, LS-CG, CL-CG, CS-CG, CLG or MFG. Miyoshi et al., (1998) J. Virol. 72(10): 8150-8157; Onishi et al. (1996) Experimental Hematology 24:324-329; Riviere et al. (1995) PNAS 92: 6733-6737. Virus vector plasmids (or constructs), include: pMXs, pMXs-IB, pMXs-puro, pMXs-neo (pMXs-IB is a vector carrying the blasticidin-resistant gene in stead of the puromycin-resistant gene of pMXs-puro) [Experimental Hematology, 2003, 31 (11): 1007-14], MFG [Proc. Natl. Acad. Sci. U.S.A. 92, 6733-6737 (1995)], pBabePuro [Nucleic Acids Research 18, 3587-3596 (1990)], LL-CG, CL-CG, CS-CG, CLG [Journal of Virology 72: 8150-8157 (1998)] and the like as the retrovirus system, and pAdex1 [Nucleic Acids Res. 23: 3816-3821 (1995)] and the like as the adenovirus system. In exemplary embodiments, the retroviral construct comprises blasticidin (e.g., pMXs-IB), puromycin (e.g., pMXs-puro, pBabePuro); or neomycin (e.g., pMXs-neo). See, e.g., Morgenstern et al. (1990) Nucleic Acids Research 18: 3587-3596.

The retroviral construct may encode one or more IFs. In an exemplary embodiment, pMX vectors encoding Oct3/4, Sox2, Klf4, or c-Myc polypeptides, or variants thereof, are generated or obtained. For example, Oct3/4 is inserted into pMXs-puro to create pMX-Oct3/4; Sox2 is inserted into pMXs-neo to create pMX-Sox2; Klf4 is inserted into pMXs-IB to create pMX-Klf4; and c-Myc is inserted into pMXs-IB to create pMX-c-Myc.

Methods of producing recombinant viruses from packaging cells and their uses are well-established, see, e.g., U.S. Pat. Nos. 5,834,256; 6,910,434; 5,591,624; 5,817,491; 7,070,994; and 6,995,009, incorporated herein by reference. Many methods begin with the introduction of a viral construct into a packaging cell line. The viral construct may be introduced by any method known in the art, including but not limited to: the calcium phosphate method [Kokai (Japanese Unexamined Patent Publication) No. 2-227075], the lipofection method [Proc. Natl. Acad. Sci. U.S.A. 84: 7413 (1987)], the electroporation method, microinjection, Fugene transfection, and the like, and any method described herein.

In one example, pMX-Oct3/4, pMX-Sox2, pMX-Klf4 or pMX-c-Myc is introduced into PlatE cells by Fugene HD (Roche) transfection. The cell culture medium may be replaced with fresh medium comprising FBM (Lonza) supplemented with FGM-2 Single Quots (Lonza). In some embodiments, the medium is replaced from about 12 to about 60 hours following the introduction of the viral construct, e.g., from about 12 to about 18 hours; about 18 to about 24; about 24 to about 30; about 30 to about 36; about 36 to about 42; about 42 to about 48; about 48 to about 54; or about 54 to about 60 hours following introduction of the viral construct to the producer cells. The medium may be replaced from about 24 to about 48 hours after introduction of the viral construct to the producer cells. The supernatant can be recovered from about 4 to about 24 hours following the addition of fresh media, e.g., about 4 hours. In some cases, the supernatant may be recovered about every 4 hours following the addition of fresh media. The recovered supernatant may be passed through a 0.45 uM filter (Millipore). In some cases, the recovered supernatant comprises retrovirus derived from one or more: pMX-Oct3/4, pMX-Sox2, pMX-Klf4 or pMX-c-Myc.

Adenoviral transduction may be used to force expression of the sets of IFs. Methods for generating adenoviruses and their use are well established as described in, e.g., Straus, The Adenovirus, Plenum Press (NY 1984), 451 496; Rosenfeld, et al, Science, 252:431-434 (1991); U.S. Pat. Nos. 6,203,975, 5,707,618, and 5,637,456. In other cases, adenoviral-associated viral transduction is used to force expression of the sets of IFs. Methods for preparing adeno-associated viruses and their use are well established as described in, e.g., U.S. Pat. Nos. 6,660,514 and 6,146,874.

In an exemplary embodiment, an adenoviral construct is obtained or generated, wherein the adenoviral construct, e.g., Adeno-X, comprises DNA encoding Oct3/4, Sox2, Klf4, or c-Myc. An adenoviral construct may be introduced by any method known in the art, e.g., Lipofectamine 2000 (Invitrogen) or Fugene HD (Roche), into HEK 293 cells. In some cases, the method further comprises (1) collecting the cells when they exhibit a cytopathic effect (CPE), such effect occurring from about 10 to about 20 days, e.g., about 11, 13, 14, 15, 18, or 20 days after transfection (2) subjecting the cells to from about 2 to about 5 freeze-thaw cycles, e.g., about 3, (3) collecting the resulting virus-containing liquid; (4) purifying the virus using an adenovirus purification kit (Clontech) and (5) storing the virus at −80° C. In some cases, the titer, or plaque-forming unit (PFU), of the adenoviral stocks is determined using an Adeno-X rapid titer kit (Clontech), as described herein.

IFs can also be delivered using nonintegrating approaches. Two such approaches, adenoviral delivery and transient transfection, have been successfully used in the reprogramming of mouse cells (Okita et al., (2008) Science 322, 949-953; Stadtfeld et al., (2008) Science 322, 945-949) and can be adapted to the purposes of the present methods. Adenoviral transduction may be used to force expression of the sets of IFs. Methods for generating adenoviruses and their use are well established as described in, e.g., Straus, The Adenovirus, Plenum Press (NY 1984), 451 496; Rosenfeld, et al., (1991), Science, 252:431-434; U.S. Pat. Nos. 6,203,975; 5,707,618; and 5,637,456. In other cases, adenoviral-associated viral transduction is used to force expression of the sets of IFs. Methods for preparing adeno-associated viruses and their use are well established as described in, e.g., U.S. Pat. Nos. 6,660,514 and 6,146,874.

In an exemplary embodiment, an adenoviral construct is obtained or generated, wherein the adenoviral construct, e.g., Adeno-X, comprises DNA encoding Oct3/4, Sox2, Klf4, or c-Myc. An adenoviral construct may be introduced by any method known in the art, e.g., Lipofectamine 2000 (Invitrogen) or Fugene HD (Roche), into HEK 293 cells. In some cases, the method further comprises (1) collecting the cells when they exhibit a cytopathic effect (CPE), such effect occurring from about 10 to about 20 days, e.g., about 11, 13, 14, 15, 18, or 20 days after transfection (2) subjecting the cells to from about 2 to about 5 freeze-thaw cycles, e.g., about 3, (3) collecting the resulting virus-containing liquid; (4) purifying the virus using an adenovirus purification kit (Clontech) and (5) storing the virus at −80° C. In some cases, the titer, or plaque-forming unit (PFU), of the adenoviral stocks is determined using an Adeno-X rapid titer kit (Clontech), as described herein.

The cells may be infected with a recombinant retrovirus that naturally targets a different cell type or cells originating from a different host. To aid infection efficiency, an exogenous receptor may be first introduced into the human cells. For example, an exogenous mouse receptor may be added to human cells, e.g., postnatal dermal fibroblasts, in order help entry of murine moloney leukemia virus (MMLV). The exogenous receptor may improve infection efficiency by facilitating viral entry, especially if the receptor recognizes a viral polypeptide, e.g., MMLV env, or HIV env. Examples of exogenous receptors include but are not limited to any receptor recognized by a specific retrovirus or lentivirus known in the art. For example, a murine receptor, mCAT1, GenBank Accession No NM_{—}007513 protein is used in order to aid MMLV infection of a human target cell. In another example, a CXCR4 or CCR5 receptor is used to aid HIV-1 infection of a target cell.

The exogenous receptor may be introduced by methods described herein. Methods of introducing the exogenous receptor include but are not limited to: calcium phosphate transfection, Lipofectamine transfection, Fugene transfection, microinjection, or electroporation. In exemplary embodiments, a virus, e.g., recombinant adenovirus or retrovirus (including lentivirus), is used to introduce the exogenous receptor to the target cell. In a further exemplary embodiment, a recombinant adenovirus is used to introduce MCAT1 to human cells and then a recombinant retrovirus, e.g., MMLV, is used to introduce the IF genes, e.g., Oct 3/4, a Sox2, a Klf4, or c-Myc, to the cells.

In some cases, a solution of adenovirus comprising DNA encoding the mCAT1 protein, e.g., an adenovirus generated by using a pADEX-mCAT1 construct, is generated or obtained. The adenovirus solution can comprise Hanks' balanced salt solution. In exemplary embodiments, infection of cells is accomplished by: (1) contacting the p-ADEX-mCAT1 adenovirus solution with cells, e.g., human, non-embryonic fibroblasts, at a multiplicity of infection (m.o.i.) from about 1:5 to about 1:50, e.g., about 1:5, about 1:7; about 1:10; about 1:15, about 1:20, about 1:25; about 1:30, about 1:35; about 1:40; about 1:45, or about 1:50; (2) incubating the cells with the adenovirus solution at room temperature from about 15 minutes to about 2 hours, e.g., about 15 minutes, about 30 minutes, about 45 minutes, about 1 hour, about 1.25 hours, about 1.5 hours, about 1.75 hours, or about 2 hours; and (3) culturing the somatic cell population in culture medium from about 24 hours to about 60 hours, e.g., about 24 hours, about 30 hours, about 36 hours, about 42 hours, about 48 hours, about 54 hours, or about 60 hours.

The cells can be infected using a wide variety of methods. In some cases, the infection of cells occurs by (1) combining one or more, two or more, three or more, or all four: pMX-Oct3/4 retrovirus, pMX-Sox2 retrovirus, pMX-Klf4, or pMX-c-Myc to obtain a retrovirus solution (2) supplementing the retrovirus solution with from about 2 ug/ml to about 15 ug/ml Polybrene, e.g., about 2 ug/ml, about 3 ug/ml, about 5 ug/ml, about 7 ug/ml, about 10 ug/ml, about 12 ug/ml, or about 15 ug/ml Polybrene; (3) contacting the retroviral solution with the somatic cells, at a m.o.i. of from about 1:100 to about 1:500, e.g., about 1:100, about 1:150, about 1:200, about 1:250, about 1:300, about 1:350, about 1:400, about 1:450, or about 1:500 m.o.i.; (4) allowing the contacting of step (3) to continue at 37° C. from about 2 hours to about 24 hours, e.g., about 2 hours, about 3 hours, about 4 hours, about 5 hours, about 6 hours, about 7 hours, about 9 hours, about 10 hours, about 11 hours, about 12 hours, about 14 hours, about 15 hours, about 16 hours, about 17 hours, about 18 hours, about 19 hours, about 20 hours, about 21 hours, about 22 hours, about 23 hours, or about 24 hours; (5) soon after the contacting of step (4), changing the medium to MC-ES medium, as described herein; and (6) changing the MC-ES medium with fresh medium every 1 to 2 days. In some cases, infection of somatic cells occurs by following steps (1) through (6) described herein, with the added step of pre-incubating the somatic cells for a length of time, e.g., about 48 hours, prior to contacting the cells with the retroviral solution. Such pre-incubation may be necessary when the somatic cell expresses an exogenous receptor that was introduced by viral transduction, transfection, or other method. Thus, in some embodiments, if an adenovirus or lentivirus is used to introduce an exogenous receptor, e.g., mCAT1, to the somatic cell; such cells may need to be cultured for a length of time from at least about 30 hours to at least about 60 hours, e.g., about 30, about 35, about 40, about 48, about 52, about 55, or about 60 hours.

The infection of cells may be accomplished by any method known in the art. e.g., Palsson, B., et al. WO95/10619. Apr. 20, 1995; Morling, F. J. et al. (1995). Gene Therapy. 2: 504-508; Gopp et al. (2006) Methods Enzymol. 420:64-81. For example, the infection may be accomplished by spin-infection or “spinoculation” methods that involve subjecting the cells to centrifugation during the period closely following the addition of virus to the cells. In some cases, virus may be concentrated prior to the infection, e.g., by ultracentrifugation. In some cases, other technologies may be used to aid or improve entry of retroviruses into the target cell. For example, the retrovirus may be contacted with a liposome or immunoliposome to aid or direct entry into a specific cell type. See, e.g., Tan et al. (2007) Mol. Med. 13(3-4): 216-226.

The methods of infecting cells described herein may be used to infect cells expressing an exogenous receptor, e.g., MCAT1 or other exogenous receptor described herein. Depending on how the exogenous receptor was introduced, the preincubation period of the cells prior to infection may need to be varied. In some cases, cells that do not express an exogenous receptor are used. Some recombinant retroviruses, e.g., VSV-G pseudotyped recombinant retroviruses, may not need the aid of an exogenous receptor in order to efficiently enter cells. In some examples, VSV-G pseudotyped recombinant retrovirus is introduced to cells following the method described herein, except that the timing of the preculturing of the cells may vary.

2. Nucleic Acid Vectors

Nucleic acid vector transfection (e.g., transient transfection) methods may be used to introduce IFs into human cells. Methods for preparation of transfection-grade nucleic acid expression vectors are well established. See, e.g., Sambrook and Russell (2001), “Molecular Cloning: A Laboratory Manual,” 3rd Ed., (CSHL Press). Examples of high efficiency transfection efficiency methods include “nucleofection,” as described in, e.g., Trompeter (2003), J Immunol Methods, 274(1-2):245-256, and in international patent application publications WO2002086134, WO200200871, and WO2002086129, transfection with lipid-based transfection reagents such as Fugene® 6 and Fugene® HD (Roche), DOTAP, and Lipofectamine™ LTX in combination with the PLUS™ (Invitrogen, Carlsbad, Calif.), Dreamfect™ (OZ Biosciences, Marseille, France), GeneJuice™ (Novagen, Madison, Wis.), polyethylenimine (see, e.g., Lungwitz et al. (2005), Eur J Pharm Biopharm, 60(2):247-266), and GeneJammer™ (Stratagene, La Jolla, Calif.), and nanoparticle transfection reagents as described in, e.g., U.S. patent application Ser. No. 11/195,066.

In some embodiments, transfection may be implemented multiple times in order to increase the efficiency of transfection. In some embodiments, the methods comprises: contacting cells with one or more compounds of the invention (e.g., Category A, B, or C compounds) and with a nucleic acid vector described, e.g., in Kaji et al. (2009) Virus-free induction of pluripotency and subsequent excision of induction factors, Nature advance online publication, 1 Mar. 2009|doi:10.1038/nature07864; Woltjen et al. (2009) piggyBac transposition reprograms fibroblasts to induced pluripotent stem cells, Nature advance online publication, 1 Mar. 2009|doi:10.1038/nature07863

H. Protein Transduction

The induction methods may use protein transduction to introduce at least one of the IFs directly into cells. In some cases, protein transduction method includes contacting cells with a composition containing a carrier agent and at least one purified polypeptide comprising the amino acid sequence of one of the above-mentioned IFs. Examples of suitable carrier agents and methods for their use include, but are not limited to, commercially available reagents such as Chariot™ (Active Motif, Inc., Carlsbad, Calif.) described in U.S. Pat. No. 6,841,535; Bioport® (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents as described in, e.g., in U.S. patent application Ser. No. 138,593.

The protein transduction method may comprise contacting a cells with at least one purified polypeptide comprising the amino acid sequence of one of the above-mentioned TAs fused to a protein transduction domain (PTD) sequence (IF-PTD fusion polypeptide). The PTD domain may be fused to the amino terminal of an IF sequence; or, the PTD domain may be fused to the carboxy terminal of an IF sequence. In some cases, the IF-PTD fusion polypeptide is added to cells as a denatured polypeptide, which may facilitate its transport into cells where it is then renatured. Generation of PTD fusion proteins and methods for their use are established in the art as described in, e.g., U.S. Pat. Nos. 5,674,980, 5,652,122, and 6,881,825. See also, Becker-Hapak et al. (2003), Curr Protocols in Cell Biol, John Wiley & Sons, Inc. Exemplary PTD domain amino acid sequences include, but are not limited to, any of the following: YGRKKRRQRRR (SEQ ID NO:1); RKKRRQRR (SEQ ID NO:2); YARAAARQARA (SEQ ID NO:3); THRLPRRRRRR (SEQ ID NO:4); and GGRRARRRRRR (SEQ ID NO:5).

In some cases, individual purified IF polypeptides are added to cells sequentially at different times. In other embodiments, a set of at least three purified IF polypeptides, but not a purified c-Myc polypeptide, e.g., an Oct3/4 polypeptide, a Sox2 polypeptide, and a Klf4 polypeptide are added to cells. In some embodiments, a set of four purified IF polypeptides, e.g., purified Oct3/4, Sox2, Klf4, and c-Myc polypeptides are added to cells. In some embodiments, the purified IF polypeptides are added to cells as one composition (i.e., a composition containing a mixture of the IF polypeptides). In some embodiments, cells are incubated in the presence of a purified IF polypeptide for about 30 minutes to about 24 hours, e.g., 1 hours, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours. In some embodiments, protein transduction of cells is repeated with a frequency of about every day to about every 4 days, e.g., every 1.5 days, every 2 days, every 3 days, or any other frequency from about every day to about every four days

Forced expression of IFs may also be achieved by using nucleic acid-free IF-containing protein transducing nanoparticles (PTN). Details of methods for generating and using PTNs are found in, e.g., Link et al. (2006), Nuc Acids Res, 34(2):e16.

In some cases, the methods described herein utilize protein transduction and expression vector transduction/transfection in any combination to force expression of a set of IFs as described herein. In some embodiments, retroviral expression vectors are used to force expression of Oct 3/4, a Sox2, and a Klf4 polypeptides in cells, and purified c-Myc purified polypeptide is introduced into cells by protein transduction as described herein. HDAC inhibitor treatment can be used in addition to the purified IF polypeptide. In some cases, a set of at least three purified IF polypeptides, but not a purified c-Myc polypeptide, e.g., an Oct3/4 polypeptide, a Sox2 polypeptide, and a Klf4 polypeptide are added to cells which are also subjected to HDAC inhibitor treatment.

### IV. Cells to be Induced

iSC cells are generated from mammalian cells (including mammalian somatic cells) using, e.g., known methods. Examples of suitable mammalian cells include, but are not limited to: fibroblasts, skin fibroblasts, dermal fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, epithelial cells, gastric epithelial cells, lung epithelial cells, synovial cells, kidney cells, skin epithelial cells, pancreatic beta cells, and osteoblasts.

Mammalian cells used to generate iPS cells can originate from a variety of types of tissue including but not limited to: bone marrow, skin (e.g., dermis, epidermis), muscle, adipose tissue, peripheral blood, foreskin, skeletal muscle, and smooth muscle. The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

iPS or iSC cells produce and express on their cell surface one or more of the following cell surface antigens: SSEA-3, SSEA-4, TRA-1-60, TRA-1-81, TRA-2-49/6E (alkaline phophatase), and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT.

The cells may be from non-embryonic tissue, e.g., at a stage of development later than the embryonic stage. In some cases, the cells may be derived from a fetus. In some cases, the cells are not from a fetus. In some cases, the cells are from an embryo. In some cases, the cells are not from an embryo.

The cells can be obtained from a single cell or a population of cells. The population may be homogenous or heterogeneous. The cells may be a population of cells found in a human cellular sample, e.g., a biopsy or blood sample. In some cases, the cells are a cell line. In some cases, the cells are somatic cells. In some cases, the cells are derived from cells fused to other cells. In some cases, the cells are not derived from cells fused to other cells. In some cases, the cells are not derived from cells artificially fused to other cells. In some cases, the cells are not: a cell that has been fused with an embryonic stem cell, or a cell that has undergone the procedure known as somatic cell nuclear transfer.

The cellular population may include both differentiated and undifferentiated cells. In some cases, the population primarily contains differentiated cells. In other cases, the population primarily contains undifferentiated cells, e.g., undifferentiated stem cells. The undifferentiated cells within the population may be induced to become pluripotent or multipotent. In some cases, differentiated cells within the cellular population are induced to become pluripotent or multipotent.

Methods for obtaining human somatic cells are well established, as described in, e.g., Schantz and Ng (2004), A Manual for Primary Human Cell Culture, World Scientific Publishing Co., Pte, Ltd. In some cases, the methods include obtaining a cellular sample, e.g., by a biopsy, blood draw, or alveolar or other pulmonary lavage. Other suitable methods for obtaining various types of human somatic cells include, but are not limited to, the following exemplary methods:

A. Bone Marrow

The donor is given a general anesthetic and placed in a prone position. From the posterior border of the ilium, a collection needle is inserted directly into the skin and through the iliac surface to the bone marrow, and liquid from the bone marrow is aspirated into a syringe. A mononuclear cell fraction is then prepared from the aspirate by density gradient centrifugation. The collected crude mononuclear cell fraction is then cultured prior to use in the methods described herein for induction pluripotency. For convenience, methods for induction of pluripotency, as described herein, are collectively referred to as “induction.”

B. Postnatal Skin

Skin tissue containing the dermis is harvested, for example, from the back of a knee or buttock. The skin tissue is then incubated for 30 minutes at 37° C. in 0.6% t sin/DMEM (Dulbecco's Modified Eagle's Medium)/F-12 with 1% antibiotics/antimycotics, with the inner side of the skin facing downward.

After the skin tissue is turned over to scrub slightly the inner side with tweezers, the skin tissue is finely cut into 1 mm2 sections using scissors, which are then centrifuged at 1200 rpm and room temperature for 10 minutes. The supernatant is removed, and to the tissue precipitate is added 25 ml of 0.1% trypsin/DMEM/F-12/1% antibiotics, antimycotics, and stirred using a stirrer at 37° C. and 200-300 rpm for 40 minutes. After confirming that the tissue precipitate is fully digested, 3 ml fetal bovine serum (FBS) (manufactured by JRH) is added, and filtered sequentially with gauze (Type I manufactured by PIP), a 100 μm nylon filter (manufactured by FALCON) and a 40 μm nylon filter (manufactured by FALCON). After centrifuging the resulting filtrate at 1200 rpm and room temperature for 10 minutes to remove the supernatant, DMEM/F-12/1% antibiotics, antimycotics is added to wash the precipitate, and then centrifuged at 1200 rpm and room temperature for 10 minutes. The cell fraction thus obtained is then cultured prior to induction.

C. Postnatal Skeletal Muscle

After the epidermis of a connective tissue containing muscle such as the lateral head of the biceps brachii muscle or the sartorius muscle of the leg is cut and the muscle tissue is excised, it is sutured. The whole muscle obtained is minced with scissors or a scalpel, and then suspended in DMEM (high glucose) containing 0.06% collagenase type IA and 10% FBS, and incubated at 37° C. for 2 hours.

By centrifugation, cells are collected from the minced muscle, and suspended in DMEM (high glucose) containing 10% FBS. After passing the suspension through a microfilter with a pore size of 40 μm and then a microfilter with a pore size of 20 μm, the cell fraction obtained may be cultured according to any method known in the art below as crude purified cells, and used for the induction of human pluripotent stem cells of the present invention.

D. Postnatal Adipose Tissue

Cells derived from adipose tissue for use in the present invention may be isolated by various methods known to a person skilled in the art. For example, such a method is described in U.S. Pat. No. 6,153,432, which is incorporated herein in its entirety. A preferred source of adipose tissue is omental adipose tissue. In humans, adipose cells are typically isolated by fat aspiration.

In one method of isolating cells derived from adipose cells, adipose tissue is treated with 0.01% to 0.5%, preferably 0.04% to 0.2%, and most preferably about 0.1% collagenase, 0.01% to 0.5%, preferably 0.04%, and most preferably about 0.2% trypsin and/or 0.5 ng/ml to 10 ng/ml dispase, or an effective amount of hyaluronidase or DNase (DNA digesting enzyme), and about 0.01 to about 2.0 mM, preferably about 0.1 to about 1.0 mM, most preferably 0.53 mM concentration of ethylenediaminetetraacetic acid (EDTA) at 25 to 50° C., preferably 33 to 40° C., and most preferably 37° C. for 10 minutes to 3 hours, preferably 30 minutes to 1 hour, and most preferably 45 minutes.

Cells are passed through nylon or a cheese cloth mesh filter of 20 microns to 800 microns, more preferably 40 microns to 400 microns, and most preferably 70 microns. Then the cells in the culture medium are subjected to differential centrifugation directly or using Ficoll or Percoll or another particle gradient. The cells are centrifuged at 100 to 3000×g, more preferably 200 to 1500×g, most preferably 500×g for 1 minute to 1 hours, more preferably 2 to 15 minutes and most preferably 5 minutes, at 4 to 50° C., preferably 20 to 40° C. and more preferably about 25° C.

The adipose tissue-derived cell fraction thus obtained may be cultured according to the method described herein as crude purified cells containing undifferentiated stem cells, and used for the induction of human pluripotent or multipotent stem cells.

E. Blood

About 50 ml to about 500 ml vein blood or cord blood is collected, and a mononuclear cell fraction is obtained by the Ficoll-Hypaque method, as described in, e.g., Kanof et al. (1993), Current Protocols in Immunology (J. E. Coligan, A. M. Kruisbeek, D. H. Margulies, E. M. Shevack, and W. Strober, eds.), ch. 7.1.1.-7.1.5, John Wiley & Sons, New York). After isolation of the mononuclear cell fraction, approximately 1×10⁷ to 1×10⁸ human peripheral blood mononuclear cells are suspended in a RPMI 1640 medium containing 10% fetal bovine serum, 100 μg/ml streptomycin and 100 units/ml penicillin, and after washing twice, the cells are recovered. The recovered cells are resuspended in RPMI 1640 medium and then plated in a 100 mm plastic petri dish at a density of about 1×10⁷ cells/dish, and incubated in a 37° C. incubator at 8% CO₂. After 10 minutes, cells remaining in suspension are removed and adherent cells are harvested by pipetting. The resulting adherent mononuclear cell fraction is then cultured prior to the induction period as described herein. In some cases, the peripheral blood-derived or cord blood-derived adherent cell fraction thus obtained may be cultured according to the method described herein as crude purified cells containing undifferentiated stem cells, and used for the induction of human pluripotent stem cells of the present invention.

### V. Induction and Culturing

During the induction process, forced expression of certain polypeptides is carried out in cultured cells for a period of time, after which the induced cells are screened for a number of morphological and gene expression properties that characterize multipotent and pluripotent stem cells. Induced cells that meet these screening criteria may then be subcloned and expanded. In some cases, the cells to be induced may be cultured for a period of time prior to the induction procedure. Alternatively, the cells to be induced may be used directly in the induction process without a prior culture period. In some embodiments, the type of cell culture medium used is the same or very similar before, during, and after the induction process. In other cases, different cell culture media are used at different points. For example, one type of culture medium may be used directly before the induction process, while a second type of media is used during the induction process. At times, a third type of culture medium is used during the induction process.

Cells may be cultured in medium supplemented with a particular serum. In some embodiments, the serum is fetal bovine serum (FBS). The serum can also be fetal calf serum (FCS). In some cases, the serum may be Human AB serum. Mixtures of serum may also be used, e.g. mixture of FBS and Human AB, FBS and FCS, or FCS and Human AB.

Culture of cells may be carried out under a low serum culture conditions prior to, during, or following induction. A “low serum culture condition” refers to the use of a cell culture medium containing a concentration of serum ranging from 0% (v/v) (i.e., serum-free) to about 5% (v/v), e.g., 0% to 2%, 0% to 2.5%, 0% to 3%, 0% to 4%, 0% to 5%, 0.1% to 2%, 0.1% to 5%, 0.1%, 0.5%, 1%, 1.2%, 1.5%, 2%, 2.5%, 3%, 3.5%, or 4%. In some embodiments, the serum concentration is from about 0% to about 2%. In some cases, the serum concentration is about 2%. In some cases, the serum concentration is preferably 2% or less. In other embodiments, cells are cultured under a “high serum condition,” i.e., greater than 5% serum to about 20% serum, e.g., 6%, 7%, 8%, 10%, 12%, 15%, or 20%. Culturing under high serum conditions may occur prior to, during, and/or after induction.

Some representative media that the cells can be cultured in include: hFib media (DMEM, 20% Earl's 199 media, 10% FBS, Glutamax, beta-mercaptoethanol (bME)); MAPC, FBM, ES, MEF-conditioned ES (MC-ES), and mTeSR™ (available, e.g., from StemCell Technologies, Vancouver, Canada), See Ludwig et al. (2006), Nat Biotechnol, 24(2):185-187. In other cases, alternative culture conditions for growth of human ES cells are used, as described in, e.g., Skottman et al. (2006), Reproduction, 132(5):691-698. hES cell media may also be prepared (according to Cowan et al., NEJM 2004) from:

467 mL KO-DMEM (Invitrogen, Inc.)

60 mL Plasmanate (Telaris, Inc.)

60 mL knock-out serum replacement (Invitrogen, Inc.)

6 mL GlutaMAX (Invitrogen, Inc.)

6 mL non-essential amino-acids (Invitrogen, Inc.)

1.2 mL 2-mercaptoethanol (Invitrogen, Inc.)

10 ng/mL bFGF (Invitrogen, Inc.)

In some embodiments, the cells are cultured in hFib media, MAPC, FBM, MC-ES, or mTeSR™ prior to and/or during the introduction of induction factors to the cells; and the cells are cultured in hES, MC-ES or mTeSR™ medium later in the induction process.

MAPC (2% FBS) Medium may comprise: 60% Dulbecco's Modified Eagle's Medium-low glucose, 40% MCDB 201, Insulin Transferrin Selenium supplement, (0.01 mg/ml insulin; 0.0055 mg/ml transferrin; 0.005 μg/ml sodium selenite), 1× linolenic acid albumin (1 mg/mL albumin; 2 moles linoneic acid/mole albumin), 1 nM dexamethasone, 2% fetal bovine serum, 1 nM dexamethasone, 10-4 M ascorbic acid, and 10 μg/mlgentamycin.

FBM (2% FBS) Medium may comprise: MCDB202 modified medium, 2% fetal bovine serum, 5 μg/ml insulin, 50 mg/ml gentamycin, and 50 ng/ml amphotericin-B.

ES Medium may comprise: 40% Dulbecco's Modified Eagle's Medium (DMEM) 40% F12 medium, 2 mM L-glutamine, 1× non-essential amino acids (Sigma, Inc., St. Louis, Mo.), 20% Knockout Serum Replacement™ (Invitrogen, Inc., Carlsbad, Calif.), and 10 μg/mlgentamycin.

MC-ES medium may be prepared as follows. ES medium is conditioned on mitomycin C-treated murine embryonic fibroblasts (MEFs), harvested, filtered through a 0.45-μM filter, and supplemented with about 0.1 mM B mercaptoethanol, about 10 ng/ml bFGF or FGF-2, and, optionally, about 10 ng/ml activin A. In some cases, irradiated MEFs are used in place of the mitomycin C-treated MEFs.

When either low or high serum conditions are used for culturing the cells, one or more growth factors such as fibroblast growth factor (FGF)-2; basic FGF (bFGF); platelet-derived growth factor (PDGF), epidermal growth factor (EGF); insulin-like growth factor (IGF); or insulin can be included in the culture medium. Other growth factors that can be used to supplement cell culture media include, but are not limited to one or more: Transforming Growth Factor beta-1 (TGF beta-1), Activin A, Noggin, Brain-derived Neurotrophic Factor (BDNF), Nerve Growth Factor (NGF), Neurotrophin (NT)-1, NT-2, or NT 3. In some cases, one or more of such factors is used in place of the bFGF or FGF-2 in the MC-ES medium or other cell culture medium.

In some cases, the concentration of growth factors in the culture media described herein (e.g., hFib media, MAPC, FBM, MC-ES, mTeSR™) is from about 2 ng/ml to about 20 ng/ml, e.g., about 2 ng/ml, 3 ng/ml, 4 ng/ml, 5 ng/ml, 6 ng/ml, 7 ng/ml, 8 ng/ml, 10 ng/ml, 12 ng/ml, 14 ng/ml, 15 ng/ml, 17 ng/ml, or 20 ng/ml. In some embodiments, the concentration of bFGF or FGF2 is from about 2 ng/ml to about 5 ng/ml; from about 5 ng/ml to about 8 ng/ml; from about 9 ng/ml to about 11 ng/ml; from about 11 ng/ml to about 15 ng/ml; or from about 15 ng/ml to about 20 ng/ml.

The growth factors may be used alone or in combination. For example, FGF-2 may be added alone to the medium; in another example, both PDGF and EGF are added to the culture medium.

In some examples, following initiation of the forced expression of genes or polypeptides (e.g., immediately after a retroviral infection period) in cells, the “induced cells” are maintained in MC-ES medium as described herein.

In some embodiments, cells are maintained in the presence of a rho, or rho-associated, protein kinase (ROCK) inhibitor to reduce apoptosis. In some cases, an inhibitor of Rho associated kinase is added to the culture medium. For example, the addition of Y-27632 (Calbiochem; water soluble) or Fasudil (HA1077: Calbiochem), an inhibitor of Rho associated kinase (Rho associated coiled coil-containing protein kinase) may be used to culture the human pluripotent and multipotent stem cells of the present invention. In some cases the concentration of Y-27632 or Fasudil, is from about 5 μM to about 20 μM, e.g., about 5 μM, 10 μM, 15 μM, or 20 μM.

The cells may be cultured for about 1 to about 12 days e.g., 2 days, 3 days, 4.5 days, 5 days, 6.5 days, 7 days, 8 days, 9 days, 10 days, or any other number of days from about 1 day to about 12 days prior to undergoing the induction methods described herein.

In some cases, the induced cells are cultured in complete ES medium in a 37° C., 5% CO₂ incubator, with medium changes about every 1 to 2 days. In some embodiments, induced the induced cells are cultured and observed for about 14 days to about 40 days, e.g., 15, 16, 17, 18, 19, 20, 23, 24, 27, 28, 29, 30, 31, 33, 34, 35, 36, 37, 38 days, or any other period from about 14 days to about 40 days prior to identifying and selecting clones comprising “induced cells” based on morphological characteristics. Morphological characteristics for identifying induced cell clones include, but are not limited to, a small cell size with a high nucleus-to-cytoplasm ratio; formation of small monolayer colonies within the space between parental cells (e.g., between fibroblasts).

The cells may be plated at a cell density of about 1×10³ cells/cm² to about 1×10⁴ cells/cm², e.g., 2×10³ cells/cm², 3.5×10³ cells/cm², 6×10³ cells/cm², 7×10³ cells/cm², 9×10³ cells/cm², or any other cell density from about 1×10³ cells/cm² to about 1×10⁴ cells/cm².

The cells can be plated and cultured directly on tissue culture-grade plastic. Alternatively, cells are plated and cultured on a coated substrate, e.g., a substrate coated with fibronectin, gelatin, Matrigel™, collagen, or laminin Suitable cell culture vessels include, e.g., 35 mm, 60 mm, 100 mm, and 150 mm cell culture dishes, 6-well cell culture plates, and other size-equivalent cell culture vessels. In some cases, the cells are cultured with feeder cells. For example, the cells may be cultured on a layer, or carpet, of MEFs.

### VI. Screening and Selection of Subject Samples

Some of the methods described herein utilize induced stem cell lines or panels of induced stem cell lines derived from subjects that meet one or more pre-determined criteria. In some cases subjects and cellular samples from such subjects may be selected for the generation of induced stem cell lines and panels of induced stem cell lines based on one or more of such pre-determined criteria. These include, but are not limited to, the presence or absence of a health condition in a subject, one or more positive diagnostic criteria for a health condition, a family medical history indicating a predisposition or recurrence of a health condition, the presence or absence of a genotype associated with a health condition, or the presence of at least one polymorphic allele that is not already represented in a panel of induced stem cell lines.

In some cases, a panel of induced stem cell lines is generated specifically from individuals diagnosed with a health condition, and from subjects that are free of the health condition. Such health conditions include, without limitation, neurodegenerative disorders; neurological disorders such as cognitive impairment, and mood disorders; auditory disease such as deafness; osteoporosis; cardiovascular diseases; diabetes; metabolic disorders; respiratory diseases; drug sensitivity conditions; eye diseases such as macular degeneration; immunological disorders; hematological diseases; kidney diseases; proliferative disorders; genetic disorders, traumatic injury, stroke, organ failure, or loss of limb.

Examples of neurodegenerative disorders include, but are not limited to, Alexander's disease, Alper's disease, Alzheimer's disease, amyotrophic lateral sclerosis, ataxia telangiectasia, Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoffs disease, Schilder's disease, subacute combined degeneration of spinal cord secondary to pernicious anaemia, schizophrenia, spinocerebellar ataxia, spinal muscular atrophy, Steele-Richardson-Olszewski disease, and tabes dorsalis.

Examples of neurological disorders include, stroke, cognitive impairment, and mood disorders.

Examples of immunological disorders include but are not limited to acquired immune deficiency, leukemia, lymphoma, hypersensitivities (allergy), autoimmune diseases, and severe combined immune deficiency.

Examples of autoimmune diseases include but are not limited to acute disseminated encephalomyelitis, addison's disease, ankylosing spondylitis, antiphospholipid antibody syndrome, autoimmune hemolytic anemia, autoimmune hepatitis, bullous pemphigoid, coeliac disease, dermatomyositis, diabetes mellitus type 1, Goodpasture's syndrome, Graves' disease, Guillain-Barré syndrome, Hashimoto's disease, idiopathic thrombocytopenic purpura, lupus erythematosus, multiple sclerosis, myasthenia gravis, pemphigus, pernicious anaemia, polymyositis, primary biliary cirrhosis, rheumatoid arthritis, Sjögren's syndrome, temporal arthritis (also known as “giant cell arthritis”), vasculitis, Wegener's granulomatosis.

Examples of cardiovascular diseases include but are not limited to aneurysm, angina, arrhythmia, atherosclerosis, cardiomyopathy, cerebrovascular accident (stroke), cerebrovascular disease, congenital heart disease, congestive heart failure, myocarditis, valve disease coronary, artery disease dilated, cardiomyopathy, diastolic dysfunction, endocarditis, high blood pressure (hypertension), hypertrophic cardiomyopathy, mitral valve prolapse, myocardial infarction (heart attack), and venous thromboembolism.

Examples of metabolic disorders include but are not limited to acid lipase disease, amyloidosis, Barth Syndrome, biotinidase deficiency, carnitine palmitoyl transferase deficiency type II, central pontine myelinolysis, metabolic diseases of muscle including muscular dystrophy, Farber's Disease, glucose-6-phosphate dehydrogenase deficiency, gangliosidoses, trimethylaminuria, Lesch-Nyhan syndrome, lipid storage diseases, metabolic myopathies, methylmalonic aciduria, mitochondrial myopathies, mucopolysaccharidoses, mucolipidoses, mucolipidoses, mucopolysaccharidoses, multiple CoA carboxylase deficiency, nonketotic hyperglycinemia, Pompe disease, propionic acidemia, type I glycogen storage disease, urea cycle disorders, hyperoxaluria, and oxalosis.

Examples of proliferative disorders include but are not limited to one or more of the following: carcinomas, sarcomas, lymphomas, leukemias, germ cell tumors, blastic tumors, prostate cancer, lung cancer, colorectal cancer, bladder cancer, cutaneous melanoma, breast cancer, endometrial cancer, and ovarian cancer.

Further examples of diseases or disorders may be found in US applications WSGR Docket Number 36588-704.201; filed on Jun. 13, 2008; First Inventor Kazuhiro Sakurada, and WSGR Docket Number 36588-707.101; filed on Jun. 13, 2008; First Inventor Kazuhiro Sakurada, which is hereby incorporated by reference. It is also anticipated that the methods of the present invention include marketing and selling products and services for the treatment of diseases and disorders including, but not limited to, those mentioned herein.

Such subjects may be identified in, e.g., gene association studies, clinical studies, and hospitals, preferably after a final diagnosis of a health condition has been made. Preferably, subjects are identified in gene association studies that include non-affected control individuals.

In other cases, iSC lines are generated from subjects screened for the presence or absence of at least one allele associated with a health condition or a predisposition for a health condition. Such alleles indicate that an individual, though not exhibiting overt symptoms of a health condition, has a high risk of developing the health condition. For example, BRCA1 have been used to indicate a high likelihood of developing breast cancer. Genotyping of subjects may be performed on samples from a number of sources, e.g., blood banks, sperm banks, gene-association studies, hospitals, clinical trials, or any other source as long as a living cellular sample can be obtained from the individual that is genotyped. While not wishing to be bound by theory, it is believed that one or more that cellular phenotypes from individuals carrying alleles associated with health conditions will exhibit abnormalities that can serve as more reliable prognostic indicators of a health condition in combination with a genotype than a genotype alone. Further, identification of specific abnormal cellular phenotypes associated with a health condition may indicate a target pathway for screening of prophylactic and therapeutic agents for the health condition.

There is an ongoing effort to identify associations between polymorphic alleles present in the human population, e.g., single polymorphisms (SNPs) and the occurrence of common health conditions, e.g., neurodegenerative diseases, psychiatric disorders, metabolic disorders, and cardiovascular diseases. Various types of polymorphic alleles can be found in the human genome as summarized in Table 1.

A number of studies have identified alleles associated with a health condition or a predisposition towards a health condition.

### VII. Induction Factor Sequences

Described herein are polypeptides comprising the amino acid sequences of IFs used in the induction methods described herein, and exogenous genes encoding such polypeptides. In some embodiments, an IF amino acid sequence is a naturally occurring amino acid sequence, e.g., that of: human or mouse Oct 3/4, human or mouse Sox2, human or mouse Klf4, or human or mouse c-Myc polypeptides. In other embodiments, the amino acid sequence of an IF is a non-naturally occurring amino acid sequence variant of an IF that is, nevertheless, functionally or structurally homologous to an IF amino acid sequence, as described herein.

Evaluating the structural and functional homology of two or polypeptides generally includes determining the percent identity of their amino acid sequences to each other. Sequence identity between two or more amino acid sequences is determined by conventional methods. See, for example, Altschul et al., (1997), Nucleic Acids Research, 25(17):3389-3402; and Henikoff and Henikoff (1982), Proc. Natl. Acad. Sci. USA, 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art will appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of another peptide. The FASTA algorithm is described by Pearson and Lipman (1988), Proc. Nat'l Acad. Sci. USA, 85:2444, and by Pearson (1990), Meth. Enzymol., 183:63. Briefly, FASTA first characterizes sequence similarity by identifying regions shared by the query sequence (e.g., any of SEQ ID NOs:6-13) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch (1970), J. Mol. Biol., 48:444-453; Sellers (1974), SIAM J. Appl. Math., 26:787), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson (1990), Meth. Enzymol., 183:63.

Also described herein are nucleic acids (e.g., exogenous genes) encoding Oct3/4, Sox2, Klf4, or c-Myc polypeptides, as described herein, that hybridize specifically under low, medium, or high stringency conditions to a probe of at least 100 nucleotides from a nucleic acid encoding the amino acid sequence any of SEQ ID NOs:6-13. Low stringency hybridization conditions include, e.g., hybridization with a 100 nucleotide probe of about 40% to about 70% GC content; at 42° C. in 2×SSC and 0.1% SDS. Medium stringency hybridization conditions include, e.g., at 50° C. in 0.5×SSC and 0.1% SDS. High stringency hybridization conditions include, e.g., hybridization with the above-mentioned probe at 65° C. in 0.2×SSC and 0.1% SDS. Under these conditions, as the hybridization temperature is elevated, a nucleic acid with a higher homology can be obtained. Such nucleic acids encoding Oct 3/4, Sox2, Klf4, or c-Myc polypeptides are useful in the forced expression of these IFs as described herein.

A number of considerations are useful to the skilled artisan in determining if a particular amino acid sequence variant of an IF is suitable for use in the methods described herein. These considerations include, but are not limited to: (1) known structure-function relationships for the IF, e.g., the presence of modular domains such as a DNA binding domain or a transactivation domain, which, in many cases, have been shown to be functionally discrete and capable of independent function; (2) the presence of amino acid sequence conservation among naturally occurring homologs (e.g., in paralogs and orthologs) of the IF, as revealed by sequence alignment algorithms as described herein. Notably, a number of bioinformatic algorithms are known in the art that successfully predict the functional effect, i.e., “tolerance” of particular amino substitutions in the amino acid sequence of a protein on its function. Such algorithms include, e.g., pMUT, SIFT, PolyPhen, and SNPs3D. For a review see, e.g., Ng and Henikoff (2006), Ann Rev Genomics Hum Genet., 7:61-80. For example, pMUT predicts with a high degree of accuracy (about 84% overall) whether a particular amino acid substitution at a given sequence position affects a protein's function based on sequence homology. See Ferrer-Costa et al., (2005), Bioinformatics, 21(14):3176-3178; Ferrer-Costa et al., (2004), Proteins, 57(4):811-819; and Ferrer-Costa et al., (2002), J Mol Biol, 315:771-786. The PMUT algorithm server is publicly available on the world wide web at: //mmb2.pcb.ub.es:8080/PMut/. Thus, for any IF polypeptide amino acid sequence, an “amino acid substitution matrix” can be generated that provides the predicted neutrality or deleteriousness of any given amino acid substitution on IF polypeptide function.

Non-naturally occurring sequence variants can be generated by a number of known methods. Such methods include, but are not limited to, “Gene Shuffling,” as described in U.S. Pat. No. 6,521,453; “RNA mutagenesis,” as described in Kopsidas et al., (2007), BMC Biotechnology, 7:18-29; and “error-prone PCR methods.” Error prone PCR methods can be divided into (a) methods that reduce the fidelity of the polymerase by unbalancing nucleotides concentrations and/or adding of chemical compounds such as manganese chloride (see, e.g., Lin-Goerke et al., (1997), Biotechniques, 23:409-412), (b) methods that employ nucleotide analogs (see, e.g., U.S. Pat. No. 6,153,745), (c) methods that utilize ‘mutagenic’ polymerases (see, e.g., Cline, J. and Hogrefe, H. H. (2000), Strategies (Stratagene Newsletter), 13:157-161 and (d) combined methods (see, e.g., Xu et al., (1999), Biotechniques, 27:1102-1108. Other PCR-based mutagenesis methods include those, e.g., described by Osuna et al., (2004), Nucleic Acids Res., 32(17):e136 and Wong et al., (2004), Nucleic Acids Res., 10;32(3):e26), and others known in the art.

Confirmation of the retention, loss, or gain of function of the amino acid sequence variants of an IF can be determined in various types of assays according to the protein function being assessed. For example, where the IF is a transcriptional activator, e.g., an Oct3/4, function is readily assessed using cell-based, promoter-reporter assays, where the reporter construct comprises one or more cognate target elements for the transactivator polypeptide to be assayed. Methods for generating promoter-reporter constructs, introducing them into cells, and assaying various reporter polypeptide activities, can be found in detail in, e.g., Current Protocols in Molecular Biology, John Wiley & Sons, N.Y. (2005), 3.16-3.17 and 9.1-9.14, respectively). Promoter activity can be quantified by measuring a property of the reporter polypeptide (e.g., enzymatic activity or fluorescence), reporter polypeptide expression (e.g., by an ELISA assay), or reporter mRNA expression (e.g., by a fluorescent hybridization technique). Suitable reporter polypeptides include, e.g., firefly luciferase, Renilla luciferase, fluorescent proteins (e.g., enhanced green fluorescent protein), β-galactosidase, β lactamase, ALP, and horseradish peroxidase.

For example, luciferase activity can be detected by providing an appropriate luminogenic substrate, e.g., firefly luciferin for firefly luciferase or coelenterazine for Renilla luciferase. Luciferase activity in the presence of an appropriate substrate can be quantified by a number of standard techniques, e.g., luminometry. See, e.g., U.S. Pat. No. 5,744,320. Fluorescent polypeptides (e.g., EGFP) can be detected and quantified in live cells by a number of detection methods known in the art (e.g., fluorimetry or fluorescence microscopy). Details of reporter assay screens in live cells using fluorescent polypeptides, including high-throughput screening methods, can be found, e.g., in U.S. Pat. No. 6,875,578.

Described herein are a number of IFs that are transcriptional activators, i.e., polypeptides that transactivate promoters containing specific target elements to which the transcriptional activator binds as a monomer, a multimer, or in a heteromeric complex with other polypeptides. Naturally occurring transcriptional activators, e.g., Klf4, are modular proteins minimally composed of two domains as follows: a DNA binding domain that dictates the genes to be targeted and an activation domain that governs the nature and the extent of the transcriptional response through interactions with the transcriptional machinery. The two domains typically operate in an independent fashion such that the DNA binding domain of one transcriptional activator, e.g., the DNA binding domain Sox2, can be attached to the transactivation domain of another transcriptional activator, e.g., Herpes VP16, to generate a fully functional, “chimeric” transcriptional activator, e.g., a chimeric Sox2 transcriptional activator as described in, e.g., Kamachi et al., (1999), Mol Cell Biol., 19(1):107-120.

In view of the guidance provided herein, a broad range of IF sequence variants (e.g., Oct3/4, Sox2, Klf4, or c-Myc sequence variants), operable in the methods described herein, can readily be identified by those of ordinary skill in the art without undue effort.

Oct3/4 Polypeptide

As referred to herein, an “Oct3/4 polypeptide” includes human Oct 3/4, mouse Oct 3/4, or any polypeptide that:

(i) includes a DNA binding domain (DBD) that binds to the human nanog gene

### Octamer Element:

5′-TTTTGCAT-3′; and

(ii) is capable of transactivating a promoter comprising one or more nanog Octamer elements. See, e.g., Kuroda et al., (2005), Mol and Cell Biol., 25(6):2475-2485.

In some embodiments, an Oct3/4 is a polypeptide having the above-mentioned functional properties, and comprising an amino acid sequence at least 70% identical to SEQ ID NO:6 corresponding to the amino acid sequence of human Oct 3/4, also known as Homo sapiens POU class 5 homeobox 1 (POU5F1; GenBank Accession No. NP_{—}002692), e.g., 75%, 80%, 85%, 90%, 91%, 92%, 95%, 97%, 99%, or any other percent identical from at least 70% to 100% identical to SEQ ID NO:6. In some embodiments, an Oct3/4 is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:6, e.g., SEQ ID NO: 6 with at least one amin amino acid substitution, deletion, or insertion. In other embodiments, an Oct-3/4 is a polypeptide having the above-mentioned functional properties comprising the amino acid sequence of SEQ ID NO:6 with up to a total of 30 amino acid substitutions, deletions, insertions, or any combination thereof, e.g., SEQ ID NO:6 with 0, 1, 2, 3, 4, 5, 6, 7, 8, 9, 10, 11, 15, 20, 25, or any other number of amino acid substitutions, deletions, insertions, or any combination thereof, from 0 to 30.

(Human Oct 3/4)
SEQ ID NO: 6
MAGHLASDFAFSPPPGGGGDGPGGPEPGWVDPRTWLSFQGPPGGPGIGP
GVGPGSEVWGIPPCPPPYEFCGGMAYCGPQVGVGLVPQGGLETSQPEGE
AGVGVESNSDGASPEP CTVTPGAVKLEKEKLEQNPEESQDIKALQKEL
EQFAKLLKQKRITLGYTQADVGLTLGVLFGKVFSQTTICRFEALQLSFK
NMCKLRPLLQKWVEEADNNENLQEICKAETLVQARKRKRTSIENRVRGN
LENLFLQCPKPTLQQISHIAQQLGLEKDVVRVWFCNRRQKGKRSSSDYA
QREDFEAAGSPFSGGPVSFPLAPGPHFGTPGYGSPHFTALYSSVPFPEG
EAFPPVSVTTLGSPMHSN

In some embodiments, an Oct3/4 is a polypeptide having the above-mentioned functional properties, and comprising an amino acid sequence at least 70% identical to SEQ ID NO:7, e.g., 75%, 80%, 85%, 90%, 91%, 92%, 95%, 97%, 99%, or any other percent identical from at least 70% to 100% identical to SEQ ID NO:7, corresponding to amino acids 138-290 of Human Oct3/4 comprising the highly conserved POU DNA binding domain. In some embodiments, an Oct3/4 is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:7, e.g., SEQ ID NO: 7 with at least one amino acid substitution, deletion, or insertion (e.g., 1 to 10 amino acid substitutions, deletions, or insertions).

(POU/DNA Binding Domain of Human Oct 3/4)
SEQ ID NO: 7
DIKALQKELEQFAKLLKQKRITLGYTQADVGLTLGVLFGKVFSQTTICR
FEALQLSFKNMCKLRPLLQKWVEEADNNENLQEICKAETLVQARKRKRT
SIENRVRGNLENLFLQCPKPTLQQISHIAQQLGLEKDVVRVWFCNRRQK
GKRSSS

Oct3/4 polypeptides, as described herein, may include naturally occurring or non-naturally occurring homologs of human Oct 3/4. Examples of naturally occurring homologs of human Oct3/4 include, but are not limited to, those listed under GenBank Accession Nos: NP 002692; NP_{—}001108427; NP_{—}001093427; NP_{—}001009178; and NP_{—}038661, or any other Oct family members that meet the above-mentioned structural and functional criteria.

Examples of non-naturally occurring homologs of human Oct 3/4, include, but are not limited to those described in, e.g., Niwa et al., (2002), Mol Cell Biol., 22(5):1526-1536; and Lunde et al., (2004), Curr. Biol., 14(1):48-55.

pMUT analysis of the human Oct3/4 amino acid sequence (SEQ ID NO:6) based on a PSI-BLAST multiple alignment encompassing 250 sequences yields an amino acid substitution matrix (ASM) as shown in Table 17. For each wild-type amino acid position in the human Oct3/4 amino acid sequence, Table 17 shows which amino acid substitutions (of 20 possible amino acids) are predicted to be deleterious (bold and underlined) or neutral (plain text) to the protein's function. Functional assays for the ability of Oct3/4 polypeptides to bind to the cognate nanog gene octamer element (described above) and to transactivate a promoter containing one or more nanog target elements are known in the art as described in, e.g., Kuroda et al., (supra); and Loh et al., (2006), Nat. Genet., 39(4):431-440.

Sox2 Polypeptide

As referred to herein, a “Sox2 polypeptide” includes human Sox2, mouse Sox2, or any polypeptide that:

(i) includes a DNA binding domain (DBD) that binds to the human nanog gene Sox element: 5′-TACAATG-3′; and

(ii) is capable of transactivating a promoter comprising one or more nanog gene promoter Sox elements. See, e.g., Kuroda et al., (2005), Mol and Cell Biol., 25(6):2475-2485.

In some embodiments, a Sox2 polypeptide is a polypeptide having the above-mentioned functional properties, and comprising the amino acid sequence at least 70% identical to SEQ ID NO:8 corresponding to the amino acid sequence of human Sox2, i.e., sex-determining region Y-box 2 protein (GenBank Accession No. NP_{—}003097), e.g., 75%, 80%, 85%, 90%, 91%, 92%, 95%, 97%, 99%, or any other percent identical from at least 70% to 100% identical to SEQ ID NO:8. In some embodiments, a Sox2 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:8, e.g., SEQ ID NO: 8 with at least one amino acid substitution, deletion, or insertion (e.g., 1 to 10 amino acid substitutions, deletions, or insertions).

In other embodiments, a Sox2 polypeptide is a polypeptide having the above-mentioned functional properties, and comprising the amino acid sequence of SEQ ID NO:8 with up to a total of 30 amino acid substitutions, deletions, insertions, or any combination thereof, e.g., SEQ ID NO:8 with 0, 1, 2, 3, 4, 5, 6, 7, 8, 9, 10, 11, 15, 20, 25, or any other number of amino acid substitutions, deletions, insertions, or any combination thereof, from 0 to 30.

(Human Sox2)
SEQ ID NO: 8
MYNMMETELKPPGPQQTSGGGGGNSTAAAAGGNQKNSPDRVKRPMNAFM
VWSRGQRRKMAQENPKMHNSEISKRLGAEWKLLSETEKRPFIDEAKRLR
ALHMKEHPDYKYRPRRKTKTLMKKDKYTLPGGLLAPGGNSMASGVGVGA
GLGAGVNQRMDSYAHMNGWSNGSYSMMQDQLGYPQHPGLNAHGAAQMQP
MHRYDVSALQYNSMTSSQTYMNGSPTYSMSYSQQGTPGMALGSMGSVVK
SEASSSPPVVTSSSHSRAPCQAGDLRDMISMYLPGAEVPEPAAPSRLHM
SQHYQSGPVPGTAINGTLPLSHM

In some embodiments, a Sox2 polypeptide is a polypeptide having the above-mentioned functional properties, and comprising an amino acid sequence at least 70% identical to SEQ ID NO:9, e.g., 75%, 80%, 85%, 90%, 91%, 92%, 95%, 97%, 99%, or any other percent identical from at least 70% to 100% identical to SEQ ID NO:9, amino acids 40-115 of Human Sox2 comprising the highly conserved High Mobility Group-Sox-TCF (HMG-Sox-TCF) motif DNA binding domain (DBD). In some embodiments, a Sox2 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:9, e.g., SEQ ID NO: 9 with at least one amino acid substitution, deletion, or insertion (e.g., 1 to 5 amino acid substitutions, deletions, or insertions).

(HMG-Sox2-TCF DBD)
SEQ ID NO: 9
RVKRPMNAFMVWSRGQRRKMAQENPKMHNSEISKRLGAEWKLLSETEKR
PFIDEAKRLRALHMKEHPDYKYRPRRK

Sox2 polypeptides, as described herein, may include naturally occurring or non-naturally occurring homologs of human Sox2. Examples of naturally occurring homologs of human Sox2 include, but are not limited to, those listed under GenBank Accession Nos: NP_{—}001098933; NP_{—}035573, ACA58281; BAA09168; NP_{—}001032751; and NP_{—}648694, or any other Sox family members that meet the above-mentioned structural and functional criteria.

Examples of non-naturally occurring homologs of human Sox2, include, but are not limited to those described in, e.g., Kamachi et al., (1999), Mol Cell Biol., 19(1):107-120.

pMUT analysis (described above) of the human Sox2 amino acid sequence (SEQ ID NO:8) based on a PSI-BLAST multiple alignment encompassing 250 sequences yields an ASM (Table 18) showing amino acid substitutions predicted to be deleterious or neutral to the protein's function. Functional assays for the ability of Sox2 polypeptides to bind to the nanog gene Sox element and to transactivate a promoter containing one or more nanog Sox elements are known in the art as described in, e.g., Kuroda et al., (supra).

Klf4 Polypeptide

As referred to herein, a “Klf4 polypeptide” includes human Klf4, mouse Klf4, or any polypeptide that:

(i) includes a zinc-finger DNA binding domain (DBD) that binds to a Klf target element, e.g., 5′-GAGGTCC-3′ OR5′-GGGGTGT-3′; and

(ii) is capable of transactivating a promoter comprising one or more of the above-mentioned target elements. See, e.g., Nakatake et al., (2006), Mol Cell Biol., 24(20):7772-7782.

In some embodiments, a Klf4 polypeptide is a polypeptide having the above-mentioned functional properties, and comprising the amino acid sequence at least 70% identical to SEQ ID NO:10 corresponding to the amino acid sequence of human Klf4, i.e., Kruppel-Like Factor 4 (GenBank Accession No. NP_{—}004226), e.g., 75%, 80%, 85%, 90%, 91%, 92%, 95%, 97%, 99%, or any other percent identical from at least 70% to 100% identical to SEQ ID NO:10. In some embodiments, a Klf4 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:10, e.g., SEQ ID NO:10 with at least one amino acid substitution, deletion, or insertion (e.g., 1 to 10 amino acid substitutions, deletions, or insertions).

In other embodiments, a Klf polypeptide is a polypeptide having the above-mentioned functional properties, and comprising the amino acid sequence of SEQ ID NO:10 with up to a total of 30 amino acid substitutions, deletions, insertions, or any combination thereof, e.g., SEQ ID NO:10 with 0, 1, 2, 3, 4, 5, 6, 7, 8, 9, 10, 11, 15, 20, 25, or any other number of amino acid substitutions, deletions, insertions, or any combination thereof, from 0 to 30.

(Human Klf4)
SEQ ID NO: 10
MAVSDALLPSFSTFASGPAGREKTLRQAGAPNNRWREELSHMKRLPPVL
PGRPYDLAAATVATDLESGGAGAACGGSNLAPLPRRETEEFNDLLDLDF
ILSNSLTHPPESVAATVSSSASASSSSSPSSSGPASAPSTCSFTYPIRA
GNDPGVAPGGTGGGLLYGRESAPPPTAPFNLADINDVSPSGGFVAELLR
PELDPVYIPPQQPQPPGGGLMGKFVLKASLSAPGSEYGSPSVISVSKGS
PDGSHPVVVAPYNGGPPRTCPKIKQEAVSSCTHLGAGPPLSNGHRPAAH
DFPLGRQLPSRTTPTLGLEEVLSSRDCHPALPLPPGFHPHPGPNYPSFL
PDQMQPQVPPLHYQELMPPGSCMPEEPKPKRGRRSWPRKRTATHTCDYA
GCGKTYTKSSHLKAHLRTHTGEKPYHCDWDGCGWKFARSDELTRHYRKH
TGHRPFQCQKCDRAFSRSDHLALHMKRHF

In some embodiments, a Klf4 polypeptide is a polypeptide having the above-mentioned functional properties, and comprising an amino acid sequence at least 70% identical to SEQ ID NO:11, e.g., 75%, 80%, 85%, 90%, 91%, 92%, 95%, 97%, 99%, or any other percent identical from at least 70% to 100% identical to SEQ ID NO:11, amino acids 382-469 of Human Klf4 comprising the highly conserved Zinc Finger motif DNA binding domain (ZF-DBD). In some embodiments, a Klf4 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:11, e.g., SEQ ID NO:11 with at least one amino acid substitution, deletion, or insertion (e.g., 1 to 5 amino acid substitutions, deletions, or insertions).

(Human Klf4-ZF-DBD)
SEQ ID NO: 11
KRTATHTCDYAGCGKTYTKSSHLKAHLRTHTGEKPYHCDWDGCGWKFAR
SDELTRHYRKHTGHRPFQCQKCDRAFSRSDHLALHMKRH

Klf4 polypeptides, as described herein, may include naturally occurring or non-naturally occurring homologs of human Klf4. Examples of naturally occurring homologs of human Klf4 include, but are not limited to, those listed under listed under GenBank Accession Nos: NP_{—}001017280, NP_{—}057354 (Klf2); AAP36222 (Klf5); NP_{—}034767; and NP_{—}446165, or any other Klf family members that meet the above-mentioned structural and functional criteria. Examples of non-naturally occurring Klf4 polypeptides include, but are not limited to, those having the above-mentioned functional properties and comprising an amino acid sequence at least 70%, e.g., 75%, 80%, 85%, 90%, or a percent from 70% to 100% identical to SEQ ID NO:10 or SEQ ID NO:11.

In some embodiments, a Klf4 polypeptide is a non-naturally occurring polypeptide having the above-mentioned functional properties.

pMUT analysis (described above) of the human Klf4 amino acid sequence (SEQ ID NO:10) based on a PSI-BLAST multiple alignment encompassing 136 sequences yields an ASM (Table 19) showing amino acid substitutions predicted to be deleterious or neutral to the protein's function. Functional assays for the ability of Klf4 polypeptides to bind to any of the above-mentioned target elements and to transactivate a promoter containing one or more of the target elements are known in the art as described in, e.g., Nakatake et al., (supra).

c-Myc Polypeptide

As referred to herein, a “c-Myc polypeptide” includes human c-Myc, mouse c-Myc, or any polypeptide that:

(i) includes a basic helix-loop-helix leucine zipper domain and binds to a target element

comprising the sequence: 5′-CACGTG-3′; or 5′-C/GACCACGTGGTG/C-3′ and

(ii) is capable of transactivating a promoter comprising one or more of the above-mentioned target elements. See, e.g., Cowling et al., (2006), Seminars in Canc. Biol., 16:242-252.

In some embodiments, a c-Myc polypeptide is a polypeptide having the above-mentioned functional properties, and comprising an amino acid sequence at least 70% identical to SEQ ID NO:12 corresponding to the amino acid sequence of human c-Myc, i.e., myelocytomatosis viral oncogene homolog (GenBank Accession No. NP_{—}002458), e.g., 75%, 80%, 85%, 90%, 91%, 92%, 95%, 97%, 99%, or any other percent identical from at least 70% to 100% identical to SEQ ID NO:12. In some embodiments, a c-Myc polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:12, e.g., SEQ ID NO:12 with at least one amino acid substitution, deletion, or insertion (e.g., 1 to 10 amino acid substitutions, deletions, or insertions).

In other embodiments, a c-Myc polypeptide is a polypeptide having the above-mentioned functional properties, and comprising the amino acid sequence of SEQ ID NO:12 with up to a total of 30 amino acid substitutions, deletions, insertions, or any combination thereof, e.g., SEQ ID NO:12 with 0, 1, 2, 3, 4, 5, 6, 7, 8, 9, 10, 11, 15, 20, 25, or any other number of amino acid substitutions, deletions, insertions, or any combination thereof, from 0 to 30.

(Human c-Myc):
SEQ ID NO: 12
MDFFRVVENQQPPATMPLNVSFTNRNYDLDYDSVQPYFYCDEEENFYQQ
QQQSELQPPAPSEDIWKKFELLPTPPLSPSRRSGLCSPSYVAVTPFSLR
GDNDGGGGSFSTADQLEMVTELLGGDMVNQSFICDPDDETFIKNIIIQD
CMWSGFSAAAKLVSEKLASYQAARKDSGSPNPARGHSVCSTSSLYLQDL
SAAASECIDPSVVFPYPLNDSSSPKSCASQDSSAFSPSSDSLLSSTESS
PQGSPEPLVLHEETPPTTSSDSEEEQEDEEEIDVVSVEKRQAPGKRSES
GSPSAGGHSKPPHSPLVLKRCHVSTHQHNYAAPPSTRKDYPAAKRVKLD
SVRVLRQISNNRKCTSPRSSDTEENVKRRTHNVLERQRRNELKRSFFAL
RDQIPELENNEKAPKVVILKKATAYILSVQAEEQKLISEEDLLRKRREQ
LKHKLEQLRNSCA

In some embodiments, a c-Myc polypeptide is a polypeptide having the above-mentioned functional properties, and comprising an amino acid sequence at least 70% identical to SEQ ID NO:13, e.g., 75%, 80%, 85%, 90%, 91%, 92%, 95%, 97%, 99%, or any other percent identical from at least 70% to 100% identical to SEQ ID NO:13, amino acids 370-454 of Human c-Myc comprising the highly conserved basic helix-loop-helix (bHLH)-leucine zipper (LZ) DNA binding domain. In some embodiments, a Klf4 polypeptide is a polypeptide having the above-mentioned functional properties, and comprising an amino acid sequence from at least 70% to less than 100% identical (e.g., 75%, 80%, 85%, 90%, 91%, 92%, 95%, 97%, 99% identical) to SEQ ID NO:13, e.g., SEQ ID NO:13 with at least one amino acid substitution, deletion, or insertion (e.g., 1 to 5 amino acid substitutions, deletions, or insertions).

(Human c-Myc bHLH-LZ domain)
SEQ ID NO: 13
KRRTHNVLERQRRNELKRSFFALRDQIPELENNEKAPKVVILKKATAYI
LSVQAEEQKLISEEDLLRKRREQLKHKLEQLRNSCA

c-Myc polypeptides, as described herein, may include naturally occurring or non-naturally occurring homologs of human c-Myc. Examples of naturally occurring homologs of human c-Myc include, but are not limited to, those listed under listed under GenBank Accession Nos: NP_{—}001005154, NP_{—}036735, NP 034979, POCON9, and NP_{—}001026123, or any other c-Myc family members that meet the above-mentioned structural and functional criteria. Examples of non-naturally occurring homologs of human c-Myc include, but are not limited to, those described in, e.g., Chang et al., (2000), Mol Cell Biol., 20:4309-4319.

pMUT analysis (described above) of the human c-Myc amino acid sequence (SEQ ID NO:12) based on a PSI-BLAST multiple alignment encompassing 250 sequences yields an ASM (Table 20) showing amino acid substitutions predicted to be deleterious or neutral to the protein's function. Functional assays for the ability of c-Myc polypeptides to bind to any of the above-mentioned target elements and to transactivate a promoter containing one or more of the target elements are known in the art as described in, e.g., Gu et al., (1993), Proc. Natl. Acad. Sci. USA, 90:2935-2939.

In some cases, any of the Oct3/4, Sox2, Klf4, or c-Myc polypeptide DNA binding domains are fused to the Herpes VP 16 transactivation domain to generate chimeric fusion proteins that can be used as induction factors in the induction methods described herein. In one embodiment the Herpes VP16 transactivation domain comprises the following amino acid sequence:

TKTLMKKDKYTLPGGLLAPGGNSMASGVGVGAGLGAGVNQRMDSYAHMN
GWSNGSYSMMQDQLGYPQHSTTAPITDVSLGDELRLDGEEVDMTPADAL
DDFDLEMLGDVESPSPGMTHDPVSYGALDVDDFEFEQMFTDALGIDDFG
G

In some embodiments, any of the Oct 3/4, Sox2, Klf4, or c-Myc polypeptides, or combinations thereof are provided as polypeptide transduction compositions for use in the induction methods described herein. Such compositions contain at least one of the following:

(i) a purified 3/4Oct3/4 polypeptide comprising a protein transduction domain at the amino or carboxy terminus;

(ii) a carrier reagent and a purified 3/4Oct3/4 polypeptide;

(iii) a purified Sox2 polypeptide comprising a protein transduction domain and the amino acid sequence of a Sox2 polypeptide;

(iv) a carrier reagent and a purified Sox2 polypeptide;

(v) a purified Klf4 polypeptide comprising a protein transduction domain;

(vi) a carrier reagent and a purified Klf4 polypeptide;

(vii) a purified c-Myc polypeptide comprising a protein transduction domain

(viii) a carrier reagent and a purified c-Myc-polypeptide

(ix) any combination of (i) to (vi) where the composition is substantially free of a purified polypeptide comprising the amino acid of a c-Myc polypeptide.

In some embodiments, the protein transduction domain is fused to the amino terminal of an IF sequence. In other embodiments, the PTD domain is fused to the carboxy terminal of an IF sequence. In some embodiments, the IF-PTD fusion polypeptide is added to cells as a denatured polypeptide, which may facilitate its transport into cells where it is then renatured. The generation of PTD fusion proteins and methods for their use are known the art as described in, e.g., U.S. Pat. Nos. 5,674,980, 5,652,122, 6,881,825. See also, Becker-Hapak et al., (2003), Curr. Protocols in Cell Biol., John Wiley & Sons, Inc. Exemplary PTD domain amino acid sequences include, but are not limited to, any of the following: YGRKKRRQRRR (SEQ ID NO:1); RKKRRQRR (SEQ ID NO:2); YARAAARQARA (SEQ ID NO:3); THRLPRRRRRR (SEQ ID NO:4); and GGRRARRRRRR (SEQ ID NO:5).

Examples of suitable carrier agents and methods for their use include, but are not limited to those described in U.S. Pat. No. 6,841,535.

### VIII. Subcloning Induced Cell Colonies

Cell colonies may be subcloned by any method known in the art. In some cases, the induced cells are cultured and observed for about 14 days to about 40 days, e.g., 15, 16, 17, 18, 19, 20, 23, 24, 27, 28, 29, 30, 31, 33, 34, 35, 36, 37, 38 days, or any other period from about 14 days to about 40 days prior to identifying and selecting clones comprising “induced cells” based on morphological characteristics. Morphological characteristics for identifying induced cell clones include, but are not limited to, a small cell size with a high nucleus-to-cytoplasm ratio; formation of small monolayer colonies within the space between parental cells (e.g., between fibroblasts).

After washing cell cultures with a physiological buffer, e.g., Hank's balanced salt solution, colonies displaying the morphological characteristics of interest are surrounded by a cloning ring to the bottom of which silicone grease has been applied. About 100 μl (or 50 μl to 150 μl) of “Detachment Medium For Primate ES Cells” (manufactured by ReproCELL, Tokyo Japan) is then added to the cloning ring and incubated at 37° C. for about 20 minutes to form a cell suspension. The cell suspension in the ring containing the detached colonies is then added to about 2 ml of MC ES medium (or other medium described herein), and plated in one well of a MEF-coated 24-well plate or other cell culture vessel of equivalent surface area. After culturing the colony-derived cells in a 5% CO₂ cell culture incubator at 37° C. for about 14 hours, the medium is replaced. Subsequently, the medium is replaced about every two days until about 8 days later when a second subculture is carried out.

In some embodiments, in the first subculture, the medium is removed, the cells are washed with Hank's balanced salt solution, and Detachment Medium For Primate ES Cells (ReproCell, Tokyo, Japan) is then added to the cells and incubated at 37° C. for 10 minutes. After the incubation, MC-ES medium (2 ml) is added to the resulting cell suspension to quench the activity of the Detachment Medium. The cell suspension is then transferred to a centrifuge tube, and centrifuged at 200×g at 4° C. for 5 minutes. The supernatant is removed, the cell pellet is resuspended in MC ES medium, and the resuspended cells are plated on four wells of a MEF-coated 24-well plate and cultured for about seven days until a second subculture is prepared.

In the second subculture, prepared by the method described above, cells are plated on a 60 mm cell culture dish coated with matrigel at a concentration of 20 mg/cm². About eight days later (approximately 5 weeks after initiating forced expression of IFs), a third subculture is prepared in which cells are plated on two matrigel-coated 60 mm cell culture dishes, one of which can subsequently be used for gene expression analysis and the other for continued passaging as described below. One of the subcultures is used for gene expression analysis, as described herein, and the other is passaged as needed to maintain a cell line derived from the induced cell clone.

### IX. Passaging and Maintaining Induced Cells

After subcloning, the induced cells may be subcultured about every 5 to 7 days. In some cases, the cells are washed with Hank's balanced salt solution, and dispase or Detachment Medium For Primate ES Cells is added, and incubated at 37° C. for 5 to 10 minutes. When approximately more than half of the colonies are detached, MC-ES medium is added to quench enzymatic activity of the detachment medium, and the resulting cell/colony suspension is transferred to a centrifuge tube. Colonies in the suspension are allowed to settle on the bottom of the tube, the supernatant is carefully removed, and MC-ES medium is then added to resuspend the colonies. After examining the size of the colonies, any extremely large ones are broken up into smaller sizes by slow up and down pipetting. Appropriately sized colonies are plated on a matrigel-coated plastic culture dish with a base area of about 3 to 6 times that before subculture.

Examples of culture media useful for culturing human pluripotent stem cells induced from undifferentiated stem cells present in a human postnatal tissue of the present invention include, but are not limited to, the ES medium, and a culture medium suitable for culturing human ES cells such as MEF-conditioned ES medium (MC-ES) or other medium described herein, e.g., mTeSR™. In some examples, the cells are maintained in the presence of a ROCK inhibitor, as described herein.

### X. Analysis of Induced Cells

Cell colonies subcultured from those initially identified on the basis of morphological characteristics may be assayed for any of a number of properties associated with pluripotent stem cells, including, but not limited to, expression of alkaline phosphatase activity, expression of ES cell marker genes, expression of protein markers, hypomethylation of Oct3/4 and Nanog promoters relative to a parental cells, long term self-renewal, normal diploid karyotype, and the ability to form a teratoma comprising ectodermal, mesodermal, and endodermal tissues.

A number of assays and reagents for detecting alkaline phosphatase activity in cells (e.g., in fixed cells or in living cells) are known in the art. In an exemplary embodiment, colonies to be analyzed are fixed with a 10% formalin neutral buffer solution at room temperature for about 5 minutes, e.g., for 2 to 5 minutes, and then washed with PBS. A chromogenic substrate of alkaline phosphatase, 1 step BCIP (5-Bromo-4-Chloro-3′-Indolyphosphate p-Toluidine Salt) and NBT (Nitro-Blue Tetrazolium Chloride) manufactured by Pierce (Rockford, Ill.) is then added and reacted at room temperature for 20 to 30 minutes. Cells having alkaline phosphatase activity are stained blue-violet.

Putative iPS cell colonies tested for alkaline phosphatase activity may be then assayed for expression of a series of human embryonic stem cell marker (ESCM) genes including, but not limited to, Nanog, TDGF1, Dnmt3b, Zfp42, FoxD3, GDF3, CYP26A1, TERT, Oct 3/4, Sox2, Sao14, and HPRT. See, e.g., Assou et al. (2007), Stem Cells, 25:961-973. Many methods for gene expression analysis are known in the art. See, e.g., Lorkowski et al. (2003), Analysing Gene Expression, A Handbook of Methods: Possibilities and Pitfalls, Wiley-VCH. Examples of suitable nucleic acid-based gene expression assays include, but are not limited to, quantitative RT-PCR (qRT-PCR), microarray hybridization, dot blotting, RNA blotting, RNAse protection, and SAGE.

In some embodiments, levels of ESCM gene mRNA expression levels in putative iPS cell colonies are determined by qRT-PCR. Putative iPS cell colonies are harvested, and total RNA is extracted using the “Recoverall total nucleic acid isolation kit for formaldehyde- or paraformaldehyde-fixed, paraffin-embedded (FFPE) tissues” (manufactured by Ambion, Austin, Tex.). In some instances, the colonies used for RNA extraction are fixed colonies, e.g., colonies that have been tested for alkaline phosphatase activity. The colonies can be used directly for RNA extraction, i.e., without prior fixation. In an exemplary embodiment, after synthesizing cDNA from the extracted RNA, the target gene is amplified using the TaqMan® PreAmp mastermix (manufactured by Applied Biosystems, Foster City, Calif.). Real-time quantitative PCR is performed using an ABI Prism 7900HT using the following PCR primer sets (from Applied Biosystems) for detecting mRNA of the above-mentioned ESCM genes: Nanog, Hs02387400_g1, Dnmt3b, Hs00171876_ml, FoxD3, Hs00255287_s1, Zfp42, Hs01938187_s1, TDGF1, Hs02339499_g1, TERT, Hs00162669_m1, GDF3, Hs00220998_m1, CYP26A1, Hs00175627_m1, GAPDH, Hs99999905_m1).

Putative iPS cell colonies may be assayed by an immunocytochemistry method for expression of protein markers including, but not limited to, SSEA-3, SSEA-4, TRA-1-60, TRA-1-81, CD9, CD24, Thy-1, and Nanog. A wide range of immunocytochemistry assays, e.g., fluorescence immunocytochemistry assays, are known as described in, e.g., Harlow et al. (1988), Antibodies: A Laboratory Manual, Cold Spring Harbor Laboratory, Cold Spring Harbor, N.Y., 353-355, and see also, The Handbook—A Guide to Fluorescent Probes and Labeling Technologies, Molecular Probes, Inc., Eugene, Oreg., (2004).

In an exemplary embodiment, expression of one or more of the above-mentioned protein markers in putative iPS cell colonies is assayed as follows. Cultured cells are fixed with 10% formaldehyde for 10 min and blocked with 0.1% gelatin/PBS at room temperature for about an hour. The cells are incubated overnight at 4° C. with primary antibodies against SSEA-3 (MC-631; Chemicon), SSEA-4 (MC813-70; Chemicon), TRA-1-60 (ab16288; abcam), TRA-1-81 (ab16289; abcam), CD9 (M-L13; R&D systems), CD24 (ALBS; abcam), Thy1 (5E10; BD Bioscience), or Nanog (MAB1997; R&D Systems). For Nanog staining, cells are permeabilized with 0.1% Triton X-100/PBS before blocking. The cell colonies are washed with PBS three times, then incubated with AlexaFluor 488-conjugated secondary antibodies (Molecular Probes) and Hoechst 33258 (Nacalai) at room temperature for 1 h. After further washing, fluorescence is detected with a fluorescence microscope, e.g., Axiovert 200M microscope (Carl Zeiss).

Expression of embryonic stem cell (ESC) marker genes in induced cell colonies may be assayed in live cells, which increases the efficiency of identifying iSC colonies following an induction method as described herein. Examples of ESC marker genes useful for identifying induced stem cell colonies include, e.g., Oct3/4, Nanog, Klf4, Lin28, Sox2, c-Myc, or TERT. In some embodiments, mRNA for one or more of these genes is detected in live cells. In other embodiments, mRNAs for two or more of the ESC marker genes is detected. In one approach, cells are contacted with one or more molecular beacon probes that hybridize to and signal the presence of one or more stem cell marker genes. Molecular beacons (MBs) are single-stranded oligonucleotide hybridization probes that form a stem-and-loop structure. The loop contains a probe sequence that is complementary to a target sequence, and the stem is formed by the annealing of complementary arm sequences that are located on either side of the probe sequence. A fluorophore is covalently linked to the end of one arm and a quencher is covalently linked to the end of the other arm. MBs do not fluoresce when they are free in solution. However, when they hybridize to a target sequence they undergo a conformational change that enables them to fluoresce brightly. The probe sequence may range in length from about 15 to about 30 nucleotides depending on the GC content of the target probe sequence. Generally, the GC content of the target probe sequence should be from about 40 to about 60%. The flanking stem sequences may range from about 5 to about 7 nucleotides with a GC content of about 75 to about 100 percent. The design of MBs and their use to detect mRNA expression in living cells is known in the art, as described in, e.g., Rhee et al. (2008), Nuc Acid Res, 36(5):e30. Useful algorithms for determining melting temperatures of an MB duplex and an MB/target duplex are known in the art. See, e.g., the “Mfold” algorithm described in Zucker (2003), Nuc Acids Res 31(13): 3406-3415, which is public available on a web server: frontend.bioinfospi.edu/applications/mfold/cgi-bin/dna-forml.cgi. See also the Hyther Server at: ozone3.chem.wayne.edu/. Typical parameters for use in these algorithms are 200 nM concentration for beacons and nucleic acid target, a folding temperature of 37° C., and ionic condition of 10 mM KCl and 5 mM MgCl2. The induced cell colonies to be evaluated may be contacted about 14 days to about 50 days after initiating induction, e.g., 14 days to 21 days, 14 days to 28 days, 20 days to 45 days, 25 days to 40 days, 30 days to 35 days, 30 days to 50 days after induction. Preferably, cells are contacted with as low a concentration of an MB and as short a period as compatible with reliably detecting a signal. In some embodiments, the concentration of an MB of about 0.1 μM to about 5 μM (for each MB), e.g., 0.1 μM to 0.5 μM, 0.2 μM to 1 μM, 0.5 μM to 2 μM, or 3 μM to 5 μM. Incubation periods with a MB may range from about 5 minutes to about two hours, e.g., 15 minutes to 30 minutes, 20 minutes to one hour, 30 minutes to 1.5 hours, 45 minutes to 2 hours, or any other time period form about 5 minutes to two hours. In some cases, MBs are introduced into the cells without the use of a transfection reagent. In other cases, a transfection reagent optimized for oligonucleotide transfection is utilized, e.g., TransIT® oligo transfection reagent kit or any other transfection reagents known in the art. In other cases, streptolysin-O is used to transiently permealize cells to allow entry of the MBs into the cells. This method is described in, e.g., Rhee et al supra and Santangelo et al. (2004), Nuc Acids Res, 32(6): e57.

In some cases, MBs are added to adherent cell cultures and cell colonies found to be positive for expression of one or more ESC marker genes are picked off the substrate as described above. In other cases, MBs are added to induced cells in suspension and ESC-positive cells are selected by FACS or any other fluorescence based sorting method. Alternatively, MBs are added to adherent induced cells, which are then dispersed prior to FACS selection. Use of FACS for selection of iSCs is particularly useful for high throughput generation of iSC lines and panels of iSC lines.

A. Methylation Analysis

In some embodiments, a characteristic of the induced cells is reduced methylation of the genomic promoters of Oct3/4 and Nanog relative to those of their parental cells. Suitable Oct3/4 promoter regions to be analyzed include, but are not limited to, the Oct3/4 proximal promoter including conserved region 1 (CR1) and the Oct3/4 promoter distal enhancer including CR4. Suitable Nanog promoter regions to be analyzed include, but are not limited to, the Nanog proximal promoter including the Oct3/4 and Sox2 binding sites. See, e.g., Rodda et al. (2005), J Biol Chem, 280:24731-24737 and Yang et al. (2005), J Cell Biochem, 96:821-830. A number of methods for the quantitative analysis of genomic DNA are known as described in, e.g., Brena et al. (2006), J Mol Med, 84(5):365-377. In an exemplary embodiment, genomic DNA isolated from putative induced cells and cells used for a comparison is isolated and treated with bisulfite. Bisulfite-treated genomic DNA is then PCR-amplified with primers containing a T7 promoter sequence. Afterwards, RNA transcripts are generated using T7 polymerase and then treated with RNAse A to generate methylation-specific cleavage products. Methylation of individual CpG sites is assessed by MALDI-TOF mass spectrometry of the cleavage products. A detailed description of the method is provided in, e.g., Ehich et al. (2005), Proc Natl Acad Sci USA, 102:15785-15790.

B. Self-Renewal Assay

One of the characteristics of stem cells is their ability to proliferate continuously without undergoing senescence. Accordingly, induced cells are assessed for their ability to be passaged continuously in vitro. In some cases, the induced cells are assayed for their ability to be passaged for at least about 30 to at least about 100 times in vitro, e.g., about 33, 35, 40, 45, 51, 56, 60, 68, 75, 80, 90, 93, 100, or any other number of passages from at least about 30 to at least about 100 passages.

In another evaluation, induced cells are assayed for their ability to proliferate for a period of about 30 days to about 500 days from initiation of forced expression of IFs in parental cells, e.g., 40 days, 50 days, 60 days, 70 days, 80 days, 100 days, 150 days, 180 days, 200 days, 250 days, 300 days, 400 days, 450 days or any other period from about 30 days to about 500 days from initiation of forced expression of IFs in the parental cells. In some embodiments, long-term self-renewal of induced cells is determined when the cells are passaged in a defined medium (e.g., mTeSR1 medium) and in the absence of feeder cells, e.g., mTeSR1 medium as described herein. In other embodiments, cells are passaged in MC-ES medium as described herein.

C. Karyotype Analysis

As another possible analysis, induced cells are assessed for diploidy and a normal, stable karyotype, e.g., stable after the cells of have been passaged for at least one year in vitro. A number of karotype analysis methods are known in the art. In some embodiments, the karyotype analysis method is multicolor FISH as described in, e.g., Bayani et al. (2004), Curr Protoc Cell Biol, Chapter 22: Unit 22.5. In other embodiments, the karyotype analysis includes a molecular karyotype analysis as described in, e.g., Vermeesch et al. (2007), Eur J Hum Genet, 15(11):1105-1114. In an exemplary embodiment, induced cells are pretreated with 0.02 μg/ml colecemid for about 2 to about 3 hours, incubated with about 0.06 to about 0.075M KCl for about 20 minutes, and then fixed with Carnoy's fixative. Afterwards, for multicolor FISH analysis, cells are hybridized with multicolor FISH probes, e.g., those in the Star*FISH© Human Multicolour FISH (M-FISH) Kit from Cambio, Ltd (Cambridge, UK).

D. Teratoma Analysis

It is generally believed that pluripotent stem cells have the ability to form a teratoma, comprising ectodermal, mesodermal, and endodermal tissues, when injected into an immunocompromised animal. Induced cells or induced pluripotent stem cells (iPS) or ES cell-like pluripotent stem cells may refer to cells having an in vitro long-term self-renewal ability and the pluripotency of differentiating into three germ layers, and the pluripotent stem cells may form a teratoma when transplanted into a test animal such as mouse.

The induced cells may be assessed for pluripotency in a teratoma formation assay in an immunocompromised animal model. The immunocompromised animal may be a rodent that is administered an immunosuppressive agent, e.g., cyclosporin or FK-506. For example, the immunocompromised animal model may be a SCID mouse. About 0.5×106 to about 2.0×106, e.g., 0.6×106, 0.8×106, 1.0×106, 1.2×106, 1.5×106, 1.7×106, or other number of induced cells from about 0.5×106 to about 2.0×106 induced cells/mouse may be injected into the medulla of a testis of a 7- to 8-week-old immunocompromised animal After about 6 to about 8 weeks, the teratomas are excised after perfusing the animal with PBS followed by 10% buffered formalin. The excised teratomas are then subjected to immunohistological analysis. One method of distinguishing human teratoma tissue from host (e.g., rodent) tissue includes immunostaining for the human-specific nuclear marker HuNu. Immunohistological analysis includes determining the presence of ectodermal (e.g., neuroectodermal), mesodermal, and endodermal tissues. Protein markers for ectodermal tissue include, but are not limited to, nestin, GFAP, and integrin β1. Protein markers for mesodermal tissue include, but are not limited to, collagen II, Brachyury, and osteocalcin. Protein markers for endodermal tissue include, but are not limited to, α-fetoprotein (αFP) and HNF3beta.

E. Global Gene Expression

In some embodiments, global gene expression analysis is performed on putative iPS cell colonies. Such global gene expression analysis may include a comparison of gene expression profiles from a putative iPS cell colony with those of one or more cell types, including but not limited to, (i) parental cells, i.e., one or more cells from which the putative iPS cell colony was induced; (ii) a human ES cell line; or (iii) an established iPS cell line. As known in the art, gene expression data for human ES cell lines are available through public sources, e.g., on the world wide web in the NCBI “Gene Expression Omnibus” database. See, e.g., Barrett et al. (2007), Nuc Acids Res, D760-D765. Thus, in some embodiments, comparison of gene expression profiles from a putative iPS colony to those of an ES cell line entails comparison experimentally obtained data from a putative iPS cell colony with gene expression data available through public databases. Examples of human ES cell lines for which gene expression data are publicly available include, but are not limited to, hE14 (GEO data set accession numbers GSM151739 and GSM151741), Sheff4 (GEO Accession Nos GSM194307, GSM194308, and GSM193409), h_ES 01 (GEO Accession No. GSM194390), h_ES H9 (GEO Accession No. GSM194392), and h_ES BG03 (GEO Accession No. GSM194391).

It is also possible to accomplish global gene expression by analyzing the total RNA isolated from one or more iPS cell lines by a nucleic acid microarray hybridization assay. Examples of suitable microarray platforms for global gene expression analysis include, but are not limited to, the Human Genome U133 plus 2.0 microarray (Affymetrix) and the Whole Human Genome Oligo Micoarray (Agilent). A number of analytical methods for comparison of gene expression profiles are known as described in, e.g., Suarez-Farinas et al. (2007), Methods Mol Biol, 377:139-152, Hardin et al. (2007), BMC Bioinformatics, 8:220, Troyanskaya et al. (2002), Bioinformatics, 18(11):1454-1461, and Knudsen (2002), A Biologist's Guide to Analysis of DNA Microarray Data, John Wiley & Sons. In some embodiments, gene expression data from cells produced by the methods described herein are compared to those obtained from other cell types including, but not limited to, human ES cell lines, parental cells, and multipotent stem cell lines. Suitable statistical analytical metrics and methods include, but are not limited to, the Pearson Correlation, Euclidean Distance, Hierarchical Clustering (See, e.g., Eisen et al. (1998), Proc Natl Acad Sci USA, 95(25): 14863-14868), and Self Organizing Maps (See, e.g., Tamayo et al. (1999), Proc Natl Acad Sci USA, 96(6):2907-2912.

### XI. Methods for Differentiating Induced Stem Cell Lines

iSC lines may be differentiated into cell-types of various lineages. Examples of differentiated cells include any differentiated cells from ectodermal (e.g., neurons and fibroblasts), mesodermal (e.g., cardiomyocytes), or endodermal (e.g., pancreatic cells) lineages. The differentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, or cardiomyocytes.

The differentiated cells derived from the induced cells may be terminally differentiated cells, or they may be capable of giving rise to cells of a specific lineage. For example, induced cells can be differentiated into a variety of multipotent cell types, e.g., neural stem cells, cardiac stem cells, or hepatic stem cells. The stem cells may then be further differentiated into new cell types, e.g., neural stem cells may be differentiated into neurons; cardiac stem cells may be differentiated into cardiomyocytes; and hepatic stem cells may be differentiated into hepatocytes. Methods for differentiating iSCs are further disclosed in WSGR docket number 36588-704.201; filed Jun. 13, 2008; first inventor Kazuhiro Sakurada, WSGR Docket Number 36588-707.101; filed on Jun. 13, 2008; First Inventor Kazuhiro Sakurada, and WSGR Docket Number 36588-702.101; filed on Jun. 13, 2008; First Inventor Kazuhiro Sakurada, which are hereby incorporated by reference in their entirety.

There are numerous methods of differentiating the induced cells into a more specialized cell type. Methods of differentiating induced cells may be similar to those used to differentiate other stem cells, particularly ES cells, MSCs, MAPCs, MIAMI, hematopoietic stem cells (HSCs). In some cases, the differentiation occurs ex vivo; in some cases the differentiation occurs in vivo.

Any known method of generating neural stem cells from ES cells may be used to generate neural stem cells from induced cells, See, e.g., Reubinoff et al. (2001) Nat. Biotechnol. 19(12):1134-40. For example, neural stem cells may be generated by culturing the induced cells as floating aggregates in the presence of noggin, or other bone morphogenetic protein antagonist, see e.g., Itsykson et al. (2005) Mol Cell Neurosci. 30(1):24-36. In another example, neural stem cells may be generated by culturing the induced cells in suspension to form aggregates in the presence of growth factors, e.g., FGF-2, Zhang et al. (2001), Nat. Biotech. (19) 1129-1133. In some cases, the aggregates are cultured in serum-free medium containing FGF-2. In another example, the induced cells are co-cultured with a mouse stromal cell line, e.g., PA6 in the presence of serum-free medium comprising FGF-2. In yet another example, the induced cells are directly transferred to serum-free medium containing FGF-2 to directly induce differentiation.

Neural stems derived from the induced cells may be differentiated into neurons, oligodendrocytes, or astrocytes. Dopaminergic neurons play a central role in Parkinson's Disease and are thus of particular interest. In order to promote differentiation into dopaminergic neurons, induced cells may be co-cultured with a PA6 mouse stromal cell line under serum-free conditions, see, e.g., Kawasaki et al. (2000) Neuron 28(1):31-40. Other methods have also been described, see, e.g., Pomp et al. (2005), Stem Cells 23(7):923-30; U.S. Pat. No. 6,395,546.

Oligodendrocytes may also be generated from the induced cells. For example, oligodendrocytes may be generated by co-culturing induced cells or neural stem cells with stromal cells, e.g., Lee et al. (2000) Nature Biotechnol 18:675-679. In another example, oligodendrocytes may be generated by culturing the induced cells or neural stem cells in the presence of a fusion protein, in which the Interleukin (IL)-6 receptor, or derivative, is linked to the IL-6 cyotkine, or derivative thereof.

Astrocytes may also be produced from the induced cells. Astrocytes may be generated by culturing induced cells or neural stem cells in the presence of neurogenic medium with bFGF and EGF, see e.g., Brustle et al. (1999) Science 285:754-756.

Induced cells may be differentiated into pancreatic beta cells by methods known in the art, e.g., Lumelsky et al. (2001) Science 292:1389-1394; Assady et al., (2001) Diabetes 50:1691-1697; D'Amour et al. (2006) Nat Biotechnol 24:1392-1401; D'Amour et al. (2005) Nat Biotechnol 23:1534-1541. The method may comprise culturing the induced cells in serum-free medium supplemented with Activin A, followed by culturing in the presence of serum-free medium supplemented with all-trans retinoic acid, followed by culturing in the presence of serum-free medium supplemented with bFGF and nicotinamide, e.g., Jiang et al. (2007) Cell Res 4:333-444. In other examples, the method comprises culturing the induced cells in the presence of serum-free medium, activin A, and Wnt protein from about 0.5 to about 6 days, e.g., about 0.5, 1, 2, 3, 4, 5, 6, days; followed by culturing in the presence of from about 0.1% to about 2%, e.g., 0.2%, FBS and activin A from about 1 to about 4 days, e.g., about 1, 2, 3, 4 days; followed by culturing in the presence of 2% FBS, FGF-10, and KAAD-cyclopamine (keto-N-aminoethylaminocaproyl dihydro cinnamoylcyclopamine and retinoic acid from about 1 to about 5 days, e.g., 1, 2, 3, 4, or 5 days; followed by culturing with 1% B27, gamma secretase inhibitor and extendin-4 from about 1 to about 4 days, e.g., 1, 2, 3, or 4 days; and finally culturing in the presence of 1% B27, extendin-4, IGF-1, and HGF for from about 1 to about 4 days, e.g., 1, 2, 3, or 4 days.

Hepatic cells or hepatic stem cells may be differentiated from the induced cells. For example, culturing the induced cells in the presence of sodium butyrate may generate hepatocytes, see e.g., Rambhatla et al. (2003) Cell Transplant 12:1-11. In another example, hepatocytes may be produced by culturing the induced cells in serum-free medium in the presence of Activin A, followed by culturing the cells in fibroblast growth factor-4 and bone morphogenetic protein-2, e.g., Cai et al. (2007) Hepatology 45(5):1229-39. In an exemplary embodiment, the induced cells are differentiated into hepatic cells or hepatic stem cells by culturing the induced cells in the presence of Activin A from about 2 to about 6 days, e.g., about 2, about 3, about 4, about 5, or about 6 days, and then culturing the induced cells in the presence of hepatocyte growth factor (HGF) for from about 5 days to about 10 days, e.g., about 5, about 6, about 7, about 8, about 9, or about 10 days.

The method may also comprise differentiating induced cells into cardiac muscle cells. In an exemplary embodiment, the method comprises culturing the induced cells in the presence of noggin for from about two to about six days, e.g., about 2, about 3, about 4, about 5, or about 6 days, prior to allowing formation of an embryoid body, and culturing the embryoid body for from about 1 week to about 4 weeks, e.g., about 1, about 2, about 3, or about 4 weeks.

In other examples, cardiomyocytes may be generated by culturing the induced cells may in the presence of LIF, or by subjecting them to other methods in the art to generate cardiomyocytes from ES cells, e.g., Bader et al. (2000) Circ Res 86:787-794, Kehat et al. (2001) J Clin Invest 108:407-414; Mummery et al. (2003) Circulation 107:2733-2740.

Examples of methods to generate other cell-types from induced cells include: (1) culturing induced cells in the presence of retinoic acid, leukemia inhibitory factor (LIF), thyroid hormone (T3), and insulin in order to generate adipoctyes, e.g., Dani et al. (1997) J. Cell Sci 110:1279-1285; (2) culturing induced cells in the presence of BMP-2 or BMP-4 to generate chondrocytes, e.g., Kramer et al. (2000) Mech Dev 92:193-205; (3) culturing the induced cells under conditions to generate smooth muscle, e.g., Yamashita et al. (2000) Nature 408: 92-96; (4) culturing the induced cells in the presence of beta-mercaptoethanol to generate keratinocytes, e.g., Bagutti et al. (1996) Dev Biol 179: 184-196; Green et al. (2003) Proc Natl Acad Sci USA 100:15625-15630; (5) culturing the induced cells in the presence of Interleukin-3(IL-3) and macrophage colony stimulating factor to generate macrophages, e.g., Lieschke and Dunn (1995) Exp Hemat 23:328-334; (6) culturing the induced cells in the presence of IL-3 and stem cell factor to generate mast cells, e.g., Tsai et al. (2000) Proc Natl Acad Sci USA 97:9186-9190; (7) culturing the induced cells in the presence of dexamethasone and stromal cell layer, steel factor to generate melanocytes, e.g., Yamane et al. (1999) Dev Dyn 216:450-458; (8) co-culturing the induced cells with fetal mouse osteoblasts in the presence of dexamethasone, retinoic acid, ascorbic acid, beta-glycerophosphate to generate osteoblasts, e.g., Buttery et al. (2001) Tissue Eng 7:89-99; (9) culturing the induced cells in the presence of osteogenic factors to generate osteoblasts, e.g., Sottile et al. (2003) Cloning Stem Cells 5:149-155; (10) overexpressing insulin-like growth factor-2 in the induced cells and culturing the cells in the presence of dimethyl sulfoxide to generate skeletal muscle cells, e.g., Prelle et al. (2000) Biochem Biophys Res Commun 277:631-638; (11) subjecting the induced cells to conditions for generating white blood cells, e.g., Rathjen et al. (1998) Reprod Fertil Dev 10:31-47; or (12) culturing the induced cells in the presence of BMP4 and one or more: SCF, FLT3, IL-3, IL-6, and GCSF to generate hematopoietic progenitor cells, e.g., Chadwick et al. (2003) Blood 102:906-915.

In some cases, sub-populations of differentiated cells may be purified or isolated. In some cases, one or more monoclonal antibodies specific to the desired cell type are incubated with the cell population and those bound cells are isolated. In other cases, the desired subpopulation of cells expresses a reporter gene that is under the control of a cell type specific promoter.

In a specific embodiment, the hygromycin B phosphotransferase-EGFP fusion protein is expressed in a cell type specific manner. The method of purifying comprises sorting the cells to select green fluorescent cells and reiterating the sorting as necessary, in order to obtain a population of cells enriched for cells expressing the construct (e.g., hygromycin B phosphotransferase-EGFP) in a cell-type-dependent manner. Selection of desired sub-populations of cells may also be accomplished by negative selection of proliferating cells with the herpes simplex virus thymidine kinase/ganciclovir (HSVtk/GCV) suicide gene system or by positive selection of cells expressing a bicistronic reporter, e.g., Anderson et al. (2007) Mol. Ther. 15(11):2027-2036.

### XII. Panels of Induced Stem Cell Lines

In some cases, the methods described herein utilize a panel of iSC lines or a panel of cells differentiated from iSC lines. A panel of iSC lines comprises multiple iSC lines, e.g., multipotent or pluripotent iSC lines, that meet certain selection criteria. Also provided herein are panels of cells differentiated from iSC lines as described herein. Such panels of differentiated cells include, but are not limited to, panels of neural stem cells, neurons, retinal cells, glial progenitor cells, glial cells, cardiac progenitor cells, cardiomyocytes, pancreatic progenitor cells, pancreatic beta cells, hepatic stem cells, hepatocytes or lung progenitor cells. In some cases, the selection criteria for inclusion of an iSC line in a panel of iSC lines are determined prior to generating the iSC lines that will constitute the panel. In other cases, the selection criteria are applied to iSC lines generated before hand, e.g., a bank of iSC lines. Selection criteria include, but are not limited to, the presence or absence of a particular health condition in an iSC donor, a positive drug response in an iSC donor, negative, positive, or adverse drug responses in an iSC donor, the presence or absence of a particular phenotype in an iSC line or in cells differentiated from the iSC line, and the presence or absence of one or more polymorphic alleles in the cell lines or their corresponding donors.

In some embodiments, where selection criteria include the presence or absence of one or more polymorphic alleles, the panel includes genetically diverse human iSC lines in which each iSC line carries at least one polymorphic allele that is unique among the iSCs to be included in the panel, e.g., 5 to 10, 20 to 50, 50 to 200, 200 to 500, 500 to 1000, 1000 to 5000, 5000 to 20000, or 20000 to 50000 polymorphic alleles that are unique within the panel of iSC lines. Such polymorphic alleles may include, e.g., a SNP allele, a promoter allele, or a protein-encoding allele. Polymorphic alles can be screened and scored for by genotyping using any of a number of known genotyping assays. In some cases, the genotyping assay is a multiplexed genotyping assay, e.g., a nucleic acid microarray assay platform such as a “SNP chip.” In some cases, the one or more polymorphic alleles are pre-selected. In some embodiments, the one or more preselected alleles are polymorphic alleles associated with a health condition or a predisposition to a health condition. Examples of polymorphic alleles associated with a health condition or a predisposition to a health condition, include, but are not limited to, polymorphic alleles associated with a neurodegenerative disorder, a neurological disorder, an eye disease, a mood disorder, a respiratory disease, a cardiovascular disease, an immunological disorder, a hematological disease, a metabolic disorder, or a drug sensitivity condition. Polymorphic alleles may include polymorphic alleles in an encoded protein or a regulatory sequence affecting the expression of the encoded protein. In some cases, the encoded protein is a drug target. Examples of drug target proteins include, but are not limited to, GPCRs, ion channels, kinases, enzymes, and transcription factors.

In other embodiments, the one or more polymorphic alleles are pre-selected based on the presence of a high degree of surrounding linkage disequilibrium in the genome, which has been proposed as a signature of genomic loci that are likely to impact many common health conditions. Methods for identifying SNPs having a high surrounding linkage disequilibrium and genes near such SNPs are described in, e.g., Wang et al. (2006), Proc Natl Acad Sci USA, 103(1):135-140.

In some cases, a panel of iSC lines includes iSC lines generated from subjects that are diagnosed as suffering from one or more health conditions. The one or more health conditions may be one or more health conditions that are common to all of the iSC donors, or they may be health conditions that are different between the iSC donors.

In certain cases, a panel of iSC lines includes iSC lines generated from subjects that are both diagnosed as suffering from a health condition and carry a polymorphic allele associated with a health condition, e.g., a polymorphic allele associated with the diagnosed health condition.

A panel of iSC lines may include iSC lines from at least about 10 individuals to at least about 50,000 individuals, e.g., 10 to 50, 20 to 100, 50 to 250, 100 to 1000, 250, to 2000, 500 to 5000, 1000 to 10,000, 2500 to 20,000, 10,000, to 30,000, 20,000 to 40,000, or 30,000 to 50,000 individuals.

A panel of iSC lines may include iSC lines from at least two ethnic groups, e.g., 3, 4, 5, 6, 7, 8, 9, 10, 12, 15, 20, 25, 30, or 50 ethnic groups. Examples of ethnic groups include, but are not limited to, Europeans, Japanese, Chinese, and the Yoruba of Nigeria.

### XIII. Methods for Use of Induced Stem Cell Lines and Panels of Induced Stem Cell Lines

The iSC lines and panels of iSC lines described herein are useful in a number of methods relating to drug discovery and development. Typically, a drug candidate compound will be evaluated in a biochemical assay (e.g., a receptor binding assay) that evaluates only a single or very few sequence variants of the drug target expressed in a patient population. Thus, such assays provide little information as to how effective the drug candidate compound is likely to be in patients that express a drug target allele that differs from the particular drug target allele that was originally screened. Along the same lines, drug candidate compounds often undergo functional cellular screens in one or few cell lines engineered to express a specific allele of the drug target, again ignoring the genetic diversity of a human patient population not only with respect to the drug target itself, but also to that of the various downstream signal transduction proteins that play a role in the response endpoint of cells to a drug. Likewise, adverse effects of candidate drug compounds (e.g., liver toxicity) are generally evaluated in inbred animal models, which are likely to be uninformative for a variable fraction of a human patient population. In contrast, drug screening in panels of genetically diverse iSC lines, as described herein, addresses the lack of genetic diversity in the prevailing drug screening models.

The panels of genetically diverse iSC lines described herein (e.g., human iSC lines) or cells differentiated from panels of genetically diverse iSC lines, as described herein, may be used to identify test compounds that act on a drug target of interest. In some embodiments, the panels of iSCs cell lines include a sufficient number of iSC lines such that at least two, e.g., at least 3, 5, 10, 20, 50, 100, or 200 polymorphic alleles of a drug target (e.g., a GPCR, ion channel, or kinase) are represented in the panel. In some embodiments, panels of iSC lines are derived from subjects diagnosed as suffering from a health condition or identified as having a predisposition to the health condition. In other embodiments, the iSC line panels comprise iSC lines each of which that has at least one polymorphic allele associated with a health condition or a predisposition to the health condition.

Drug targets for many health conditions are known. Such drug targets may include, but are not limited to, receptors, GPCRs, growth factor receptors, neurotransmitter receptors, ion channels, enzymes, protein kinases, proteases, cytoskeletal proteins, and transcription factors. Test compounds can be assayed for their effect on a drug target by a number of assays known in the art. Such assays include cell-based assays including, but not limited to, assays for determining second messenger levels, e.g., intracellular calcium, cAMP, cGMP, arachidonic acid, and inositol phosphates; channel currents; apoptosis; proliferation; morphological changes; changes in adhesion. Examples of cell-based assays include, but are not limited to those described in, U.S. Pat. Nos. 7,319,009, 7,288,368, and 7,238,213, Cell based assays may also include determining the cellular localization of one or more proteins (e.g., protein kinases, receptors, and transcription factors) in cells in the presence or absence of a test compound. Test compounds may also be screened for their ability to alter a gene expression profile by any gene expression profiling method known in the art. In some cases, the cells to be screened may be genetically modified to express one or more reporter proteins that can indicate activation of a signaling pathway. For example protein-protein interactions between fusion proteins introduced into cells may be detected by a number of methods known in the art, e.g., by fluorescence resonance energy transfer (FRET) or enzyme fragment complementation.

Assays of drug candidate compounds in an iSC line or a panel of iSC lines can include determining a dose-response. In some embodiments, the dose response of an iSC line or that of one or more types of cells differentiated from the iSC line provides an indication that of the likely efficacy of the compound in the corresponding iSC donor. In some embodiments, the fraction of iSC lines in a panel of iSC lines that exhibit an acceptable dose-response to a test compound indicates an expected probability of an acceptable dose-response relationship in the target patient population of interest. In some cases, cell-based assays of drug candidate include a comparison of responses obtained in a panel of iSC lines or iSC-derived cells to one or more reference iSC lines or cells that serve as a positive or negative control for the effect of a drug candidate compound. The reference iSC lines or cells may be from a healthy iSC donor, from an iSC donor diagnosed as suffering from a health condition, or an iSC donor carrying a polymorphic allele associated with a health condition. In other embodiments, assays of drug candidate compounds in an iSC line or a panel of iSC lines can include determining effective concentrations, maximum tolerated dose and minimum effective concentration. Additional methods and assays are disclosed in US application WSGR Docket Number 36588-707.101; filed Jun. 13, 2008; First Inventor Kazuhiro Sakurada, hereby incorporated by reference.

In some cases, the drug screening may be conducted on cells differentiated from induced cells. Examples of such differentiated cells are described herein (e.g., hepatic cells, neural stem cells, neurons, pancreatic beta cells, cardiomyocytes, hepatic stem cells, oligodendrocytes). The drugs may be targeted to treat a specific disease or condition, e.g., a disease or condition described herein. For example, the induced cells may be differentiated into dopaminergic neurons, which are used to screen drugs for Parkinson's disease. In other cases, neurons or neural stem cells differentiated from induced cells may be used to screen drugs for treating Alzheimer's disease, multiple sclerosis, or other neurological disorders. In other cases, the induced cells may be transplanted directly into an immunocompromised animal, e.g., SCID mouse, which is then used to establish in vitro or in vivo assay systems that mimic physiologic conditions in humans or other animals. The in vitro or in vivo assay systems may be used to screen for drugs, e.g., drugs for Parkinson's disease, or as a means to identify biological mechanisms.

Screening of test compounds may also be conducted in iSC-derived cells when an abnormal cellular phenotype (e.g., abnormal cell morphology, gene expression, or signaling), associated with a health condition or a predisposition to the health condition is known, but a drug target has not yet been identified. Such assays may include contacting a test population of iSC-derived cells from one or more iSC donors with a test compound and contacting with a negative control compound a negative control population of iSC-derived cells from the same one or more iSC donors. The assayed cellular phenotype associated with the health condition of interest in the test and negative control populations can then be compared to a normal cellular phenotype. Where the assayed cellular phenotype in the test population is determined as being closer to a normal cellular phenotype than that exhibited by the negative control population, the drug candidate compound is identified as normalizing the phenotype. A normal cellular phenotype with respect to a particular health condition or a predisposition for a health condition may be established in iSC-derived cells from iSC donors that do not suffer from the health condition or a predisposition for the health condition.

Test compounds identified as lead compounds may be tested on a panel of iSC-derived cells in a manner analogous to a clinical trial. In some cases, the efficacy of the lead compound versus a negative control compound, e.g., a placebo compound is determined in a panel of iSC-derived cells from patients suffering from the same health condition. Preferably, such a panel of iSC-derived cells is from subjects that are genetically diverse. For example, such patients may be carry at least one polymorphic allele that is unique among the iSC-derived cells to be included in the panel, e.g., 5 to 10, 20 to 50, 50 to 200, 200 to 500, 500 to 1000, 1000 to 5000, 5000 to 20000, or 20000 to 50000 polymorphic alleles that are unique within the panel of iSC lines. A number of methods for quantifying the genetic diversity of a population are known in the art, e.g., the analysis of molecular variance (AMOVA) and generalized analysis of molecular variance (GAMOVA). See, e.g., Excoffier et al. (1992), Genetics, 131:479-491; Nievergelt et al. (2008), PLOS Genetics, 3(4):e51. Various clinical experimental designs known in the art may be used for comparing the effect of a lead compound versus a negative control compound. See, e.g., Chow et al. (2004) “Design and Analysis of Clinical Trials: Concepts and Methodologies,” John Wiley & Sons, Inc., Hoboken, N.J.

The efficacy of the lead compound in iSC-derived cells may be determined based on any cellular response endpoint, e.g., a response obtained in any of the cell-based assays or gene expression profiling assays mentioned herein.

In some cases, potential adverse effects of a lead compound are tested on a panel of iSC-derived cells. The iSC-derived cells may include any cell type that hepatocytes, cardiomyocytes, neurons,

Drug candidate compounds may be individual small molecules of choice (e.g., a lead compound from a previous drug screen) or in some cases, the drug candidate compounds to be screened come from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Indeed, theoretically, the systematic, combinatorial mixing of 100 interchangeable chemical building blocks results in the synthesis of 100 million tetrameric compounds or 10 billion pentameric compounds. See, e.g., Gallop et al. (1994), J. Med. Chem. 37(9), 1233. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90: 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

### EXAMPLES

### Example 1

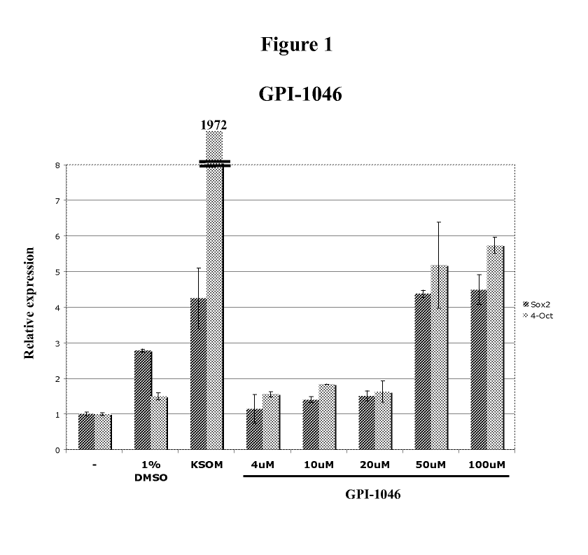

Neonatal human dermal fibroblasts (fibroblast cell line NHDF-3955-Neo from Lonza) are cultured under standard cell culture conditions using hFib media (DMEM, 20% Earl's 199 media, 10% FBS, Glutamax, beta-mercaptoethanol (bME)). Approximately 12,000 cells per well are plated on a 12-well plate. 24 hrs after plating, cells are daily fed with media containing 4, 10, 20, 50 or 100 μM GPI-1046 (ACC Corporation) or 1% DMSO. After 6 days, cells are collected and RNA is extracted using a Qiagen RNeasy kit. Gene expression is analyzed using specific Taqman primer-probe sets for Sox2, Oct4, and GAPDH on an ABI 7900HT. Gene expression of cells previously infected with retroviruses encoding Sox2, Oct4, Klf4, and c-Myc (“KSOM”) is also analyzed and is used for positive control purposes. The expression of Sox2 and Oct4 is normalized to the expression of housekeeping gene GAPDH. As indicated in FIG. 1, cells treated with 50 μM or 100 μM GPI-1046 display a 4.4 and 4.5-fold increase in Sox2 expression, respectively, compared to cells that receive no drug treatment; and about a 1.6-fold increase of relative Sox2 expression when compared to the relative expression of cells that are treated with DMSO. As also indicated in FIG. 1, cells that are treated with 50 μM or 100 μM GPI-1046 express about 5.2-fold and 5.7-fold, respectively, increases in Oct4 when compared to cells that receive no agent treatment and approximately a 3.5- and 3.8-fold increase, respectively, in relative expression of Oct4 when compared to cells that are treated with DMSO. Furthermore, cells treated with 4 μM, 10 μM, or 20 μM GPI-1046 express higher levels of Sox2, Oct3/4 or both than do cells that are treated with no agent, see FIG. 1.

### Example 2

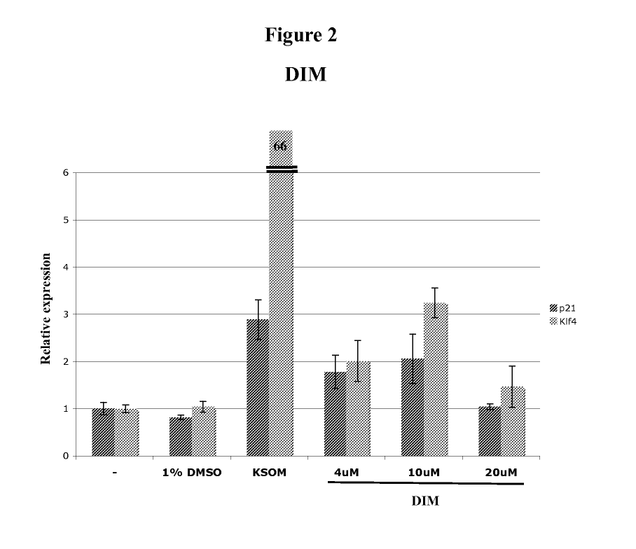

Neonatal human dermal fibroblasts (fibroblast cell line NHDF-3955-Neo from Lonza) are cultured under standard cell culture conditions using hFib media (DMEM, 20% Earl's 199 media, 10% FBS, Glutamax, beta-mercaptoethanol (bME)). Approximately 12,000 cells per well are plated on a 12-well plate. 24 hrs after plating, cells are daily fed with media containing 4, 10 or 20 μM DIM (Sigma) or 1% DMSO. After 6 days, cells are collected and RNA is extracted using a Qiagen RNeasy kit. Gene expression is analyzed using specific Taqman primer-probe sets for Klf4, p21 and GAPDH on an ABI 7900HT. Gene expression of cells previously infected with retroviruses encoding Sox2, Oct4, Klf4, and c-Myc (“KSOM”) is also analyzed and is used for positive control purposes. The expression of Klf4 and p21 is normalized to the expression of housekeeping gene GAPDH. As indicated in FIG. 2, cells that are treated with 4 μM DIM display approximately 2-fold increases in p21 and Klf4 expression relative to cells that are treated with no agent; and cells that are treated with 10 μM DIM display approximately a 2-fold increase in p21 expression and approximately a 3.2-fold increase in Klf4 expression compared to cells that are treated with no agent.

### Example 3

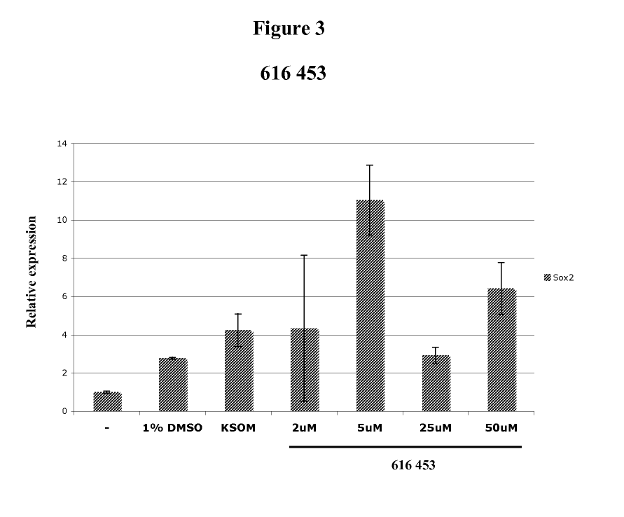

Neonatal human dermal fibroblasts (fibroblast cell line NHDF-3955-Neo from Lonza) are cultured under standard cell culture conditions using hFib media (DMEM, 20% Earl's 199 media, 10% FBS, Glutamax, beta-mercaptoethanol (bME)). Approximately 12,000 cells per well are plated on a 12-well plate. 24 hrs after plating, cells are daily fed with media containing 2, 5, 25 or 50 μM 616 453 (Calbiochem) or 1% DMSO. After 6 days, cells are collected and RNA is extracted using a Qiagen RNeasy kit. Gene expression is analyzed using specific Taqman primer-probe sets for Sox2 and GAPDH on an ABI 7900HT. Gene expression of cells previously infected with retroviruses encoding Sox2, Oct4, Klf4, and c-Myc (“KSOM”) is also analyzed and is used for positive control purposes. The expression of Sox2 is normalized to the expression of housekeeping gene GAPDH. As indicated in FIG. 3, cells that are treated with 2, 5 and 50 μM 616 453 display an increase in relative Sox2 expression when compared to cells treated with DMSO, while cells that are treated with 2, 5, 25 or 50 μM 616 453 display an increase in Sox2 expression when compared to cells that receive no treatment.

### Example 4

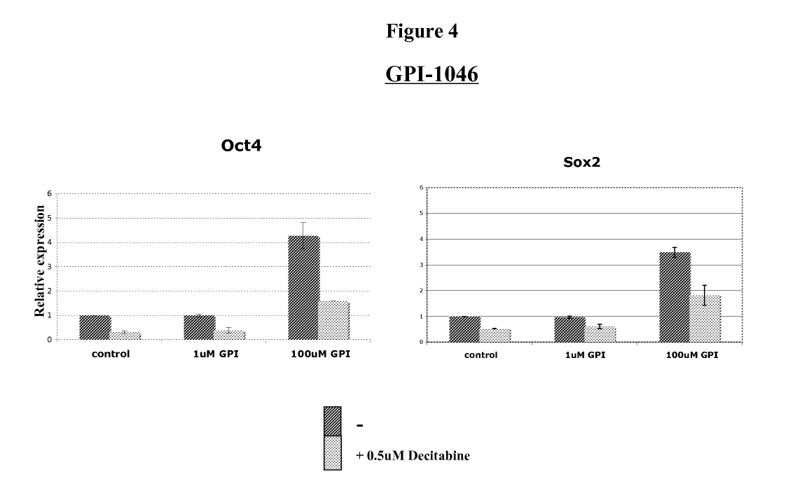

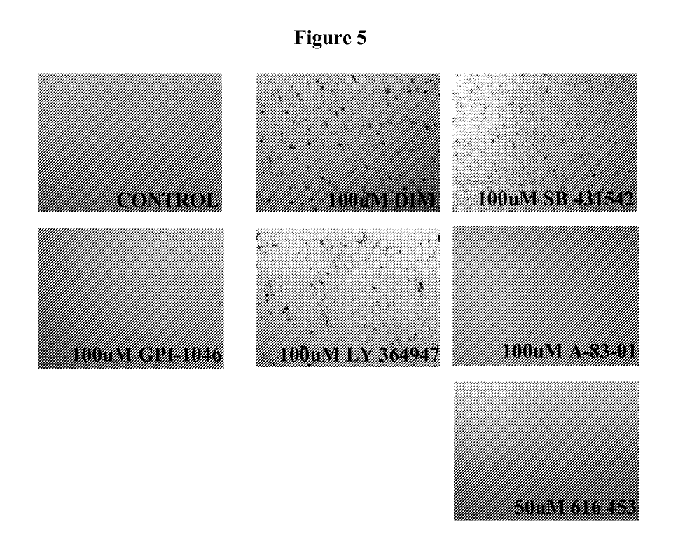

Neonatal human dermal fibroblasts (fibroblast cell line NHDF-3955-Neo from Lonza) are cultured under standard cell culture conditions using hFib media (DMEM, 20% Earl's 199 media, 10% FBS, Glutamax, beta-mercaptoethanol (bME)). Approximately 6,000 cells per well are plated on a 24-well plate. 48 hrs after plating, cells are daily fed with media containing 1 or 100 μM GPI-1046 with or without 0.5 μM Decitabine. On day 7 of treatment, cells are collected and RNA is extracted using a Qiagen RNeasy kit. Gene expression is analyzed using specific Taqman primer-probe sets for Sox2, Oct4 and GAPDH on an ABI 7900HT. The expression of Sox2 and Oct4 is normalized to the expression of housekeeping gene GAPDH. As indicated in FIG. 4, cells treated with 100 μM GPI-1046 and no Decitabine display about a 4.3-fold increase in Oct4 expression compared to control cells and about a 3.5-fold increase in Sox2 expression compared to control cells. Furthermore, cells treated with 100 μM GPI-1046 and 0.5 μM Decitabine display slightly increased expression of both Oct4 and Sox2 when compared to control cells. The morphology of cells treated with GPI-1046 is provided in FIG. 5.

### Example 5

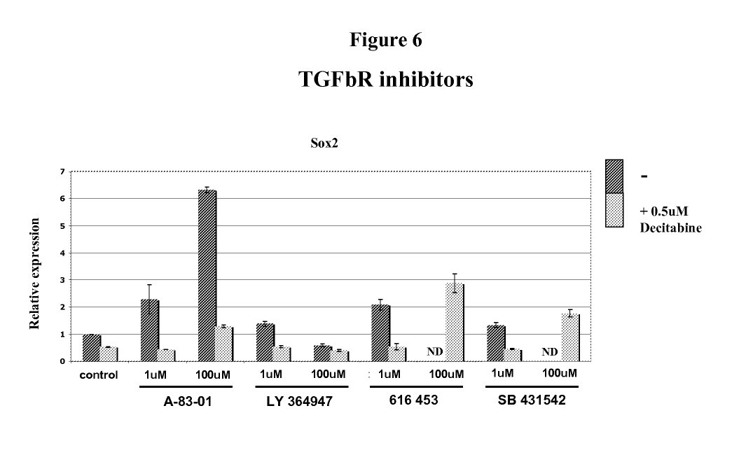

Neonatal human dermal fibroblasts (fibroblast cell line NHDF-3955-Neo from Lonza) are cultured under standard cell culture conditions using hFib media (DMEM, 20% Earl's 199 media, 10% FBS, Glutamax, beta-mercaptoethanol (bME)). Approximately 6,000 cells per well are plated on a 24-well plate. 48 hrs after plating, cells are daily fed with media containing 1 or 100 μM LY 364947 (Tocris); for 50 μM 616 453 (Calbiochem); 1 or 100 μM of A-83-01 (Tocris) or 1 or 100 μM SB 431542 (Tocris). Each treatment was performed in the presence or absence of 0.5 μM Decitabine. On day 7 of treatment, cells are collected and RNA is extracted using Qiagen RNeasy kit. Gene expression is analyzed using specific Taqman primer-probe sets for Sox2 and GAPDH on an ABI 7900HT. The expression of Sox2 is normalized to the expression of housekeeping gene GAPDH. As indicated in FIG. 6, cells treated with A-83-01 display increased relative expression of Sox2 compared to control cells in the absence of Decitabine and a slight increase of relative Sox2 expression in the presence of Decitabine. Cells that are treated with 1 μM 616 453 display an increase in relative Sox2 expression, as do cells treated with 50 μM 616 453 plus Decitabine as well as cells that are treated with 100 μM SB 431542 plus Decitabine. The morphology of the cells after treatment is depicted in FIG. 5.

### Example 6

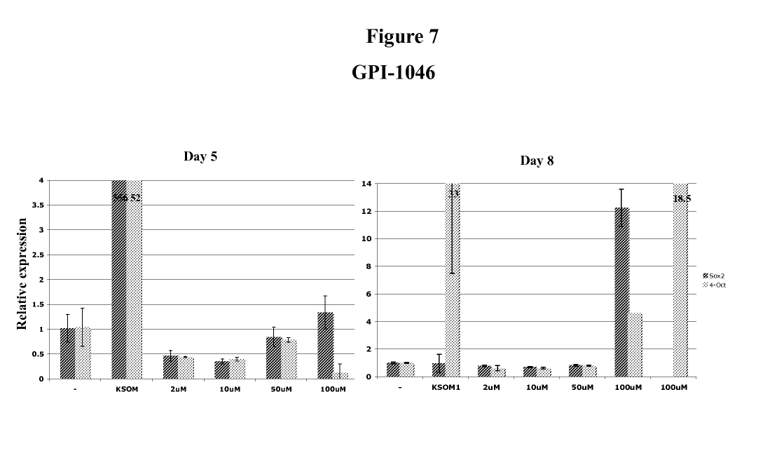

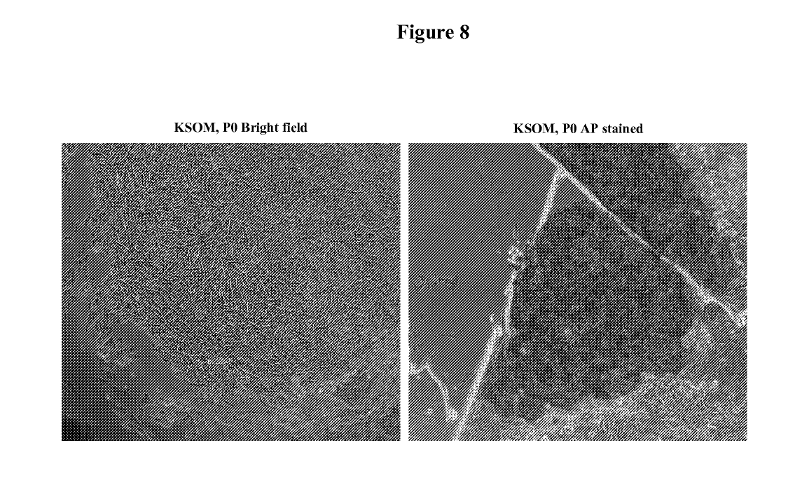

Neonatal human dermal fibroblasts (fibroblast cell line NHDF-3955-Neo from Lonza) are cultured under standard cell culture conditions using hES medium, as described herein. Cells are treated with 0 μM, 2 μM, 10 μM, 50 μM, or 100 μM GPI-1046. On day 5 or day 8 after treatment, cells are collected and RNA is extracted using a Qiagen RNeasy kit. Gene expression is analyzed using specific Taqman primer-probe sets for Sox2, Oct4 and GAPDH on an ABI 7900HT. Gene expression of cells previously infected with retroviruses encoding Sox2, Oct4, Klf4, and c-Myc (“KSOM”) is also analyzed and is used for positive control purposes. The expression of Sox2 and Oct4 is normalized to the expression of housekeeping gene GAPDH. As indicated in FIG. 7, on Day 8, cells that are treated with 100 μM GPI-1046 exhibit increases in relative Sox2 and Oct4 expression compared to cells that do not receive drug treatment. Similarly, on Day 5, cells that are treated with 100 μM GPI-1046 exhibit an increase in relative Sox2 expression when compared to control cells. The morphology of cells infected with viruses encoding KSOM is depicted in FIG. 8.

### Example 7

### Prophetic Example

Neonatal human dermal fibroblasts (fibroblast cell line NHDF-3955-Neo from Lonza) are cultured under standard cell culture conditions using hES medium. Approximately 12,000 cells per well are plated on a 12-well plate. 24 hrs after plating, cells are daily fed with media containing (1) 1 μM GPI-1046 (ACC Corporation) and 10 μM DIM (Sigma); (2) 5 μM GPI-1046 and 10 μM DIM; (3) 10 μM GPI-1046 and 10 μM DIM; (4) 50 μM GPI-1046 and 10 μM DIM; (5) 100 μM GPI-1046 and 10 μM DIM; (6) 1 μM GPI-1046; (7) 5 μM GPI-1046; (8) 10 μM GPI-1046; (9) 50 μM GPI-1046; (10) 100 μM GPI-1046; (11) 50 μM GPI-1046, 10 μM DIM and 10 μM A-83-01, (12) 50 μM GPI-1046, 10 μM DIM and 50 μM A-83-01, or (5) 1% DMSO. In a parallel experiment, di-Bromo-DIM derivative is used instead of DIM. After 6 days, cells are collected from duplicate wells and RNA is extracted using Qiagen RNeasy kit. Gene expression is analyzed using specific Taqman primer-probe sets for Sox2, Oct4, Klf4, p21 and GAPDH on ABI 7900HT. Gene expression of cells previously infected with retroviruses encoding Sox2, Oct4, Klf4, and c-Myc (“KSOM”) is also analyzed and is used for positive control purposes. On remaining duplicate wells colonies begin to appear approximately 2 weeks after introduction of the chemical compounds. About 1 month after introduction of the chemical compounds, colonies are picked and denoted Passage 0. The colonies are analyzed for pluripotency using any known method in the art, including but not limited to: testing for expression of pluripotency markers, as described herein; testing for long-term self-renewal capabilities, as described herein; or analyzing teratoma-forming ability, as described herein.

### Example 8

### Prophetic Example

About 40000 human fibroblasts per well are plated on a 6-well plate and grown in hFIB media (DMEM, 20% Earl's 199 media, 10% FBS, Glutamax, beta-mercaptoethanol (bME)) in the presence of 0 μM GPI-1046, 1 μM GPI-1046, 5 μM GPI-1046, 10 μM GPI-1046, 20 μM GPI-1046, 30 μM GPI-1046, 50 μM GPI-1046, or 100 μM GPI-1046, as described herein.

About 0 to 8 days after adding the GPI-1046, spent media is aspirated from human fibroblast plates which are then washed with 3 mL sterile PBS per well of a 6-well plate.

In a representative transduction protocol for the induction of pluripotency, viral stocks are thawed to room temperature and supplemental media prepared by adding 100× Hepes to the needed volume of growth media (DMEM, 10% FBS, beta-mercaptoethanol).

Viral cocktails are prepared such that an approximate MOI of ˜50 virus units per cell (˜5 for the transduction of c-Myc) can be conveniently applied to the wells. Cocktails include: (1) Klf4 alone; (2) Klf4 and Sox2; (3) Klf4 and c-Myc; (4) Klf4 and Oct4; (5) Sox2, Klf4, and c-Myc; or (6) Oct4, Klf4 and c-Myc. The viral cocktails that are prepared should also contain: 0 μM GPI-1046, 1 μM GPI-1046, 5 μM GPI-1046, 10 μM GPI-1046, 20 μM GPI-1046, 30 μM GPI-1046, 50 μM GPI-1046, or 100 μM GPI-1046, as described herein. Each pipette that contacts virus is washed with bleach by drawing bleach solution to top of pipette and dispensing it back into the bleach bottle, followed by dispensing in an in-hood biohazard waste receptacle.

Spent media is aspirated from the human fibroblast plate and polybrene is added to the viral cocktail (8 μg/mL final concentration), which is added onto wells of fibroblast cells. The plates are returned to the incubator and transduction allowed to proceed for about 18 to 36 hours, depending upon the viral stock, transgene, envelope and cell type.

Following transduction, viral cocktail is removed with a fresh 2 mL pipette per well of cells and dispensed into 100% bleach solution to inactivate. 2 mL of fresh hFIB media (containing 0 μM GPI-1046, 1 μM GPI-1046, 5 μM GPI-1046, 10 μM GPI-1046, 20 μM GPI-1046, 30 μM GPI-1046, 50 μM GPI-1046, or 100 μM GPI-1046) per well of cells is added and the plates replaced in the incubator for 48 hours. The media is thereafter replaced with hES cell media prepared (according to Cowan et al. NEJM 2004) from:

### 467 mL KO-DMEM (Invitrogen, Inc.)

### 60 mL Plasmanate (Telaris, Inc.)

60 mL knock-out serum replacement (Invitrogen, Inc.)

### 6 mL GlutaMAX (Invitrogen, Inc.)

6 mL non-essential amino-acids (Invitrogen, Inc.)
1.2 mL 2-mercaptoethanol (Invitrogen, Inc.)
10 ng/mL bFGF (Invitrogen, Inc.)

The hES cell media also contain: 0 μM GPI-1046, 20 μM GPI-1046, 30 μM GPI-1046, 50 μM GPI-1046, or 100 μM GPI-1046. Following media replacement, the cells are returned to the incubator. The media is replaced with fresh media daily, and each replacement also contains the corresponding level of GPI-1046. From the second feeding on, bleaching of the aspirating pipette is no longer necessary.

Colonies begin to appear approximately 2 weeks after viral transduction. About 1 month after viral transduction, colonies are picked and denoted Passage 0. The colonies are analyzed for pluripotency using any known method in the art, including but not limited to: testing for expression of pluripotency markers, as described herein; testing for long-term self-renewal capabilities, as described herein; or analyzing teratoma-forming ability, as described herein.

### Example 9

### Prophetic Example

About 40000 human fibroblasts per well are plated on a 6-well plate and grown in hFIB media (DMEM, 20% Earl's 199 media, 10% FBS, Glutamax, beta-mercaptoethanol (bME)) in the presence of 0 μM DIM or di-Bromo-DIM, 1 μM DIM or di-Bromo-DIM, 5 μM DIM or di-Bromo-DIM, 10 μM DIM or di-Bromo-DIM, 20 μM DIM or di-Bromo-DIM, 30 μM DIM or di-Bromo-DIM, 50 μM DIM or di-Bromo-DIM, or 100 μM DIM or di-Bromo-DIM, as described herein.

About 0 to 8 days after adding the DIM or di-Bromo-DIM, spent media is aspirated from human fibroblast plates which are then washed with 3 mL sterile PBS per well of a 6-well plate.

In a representative transduction protocol for the induction of pluripotency, viral stocks are thawed to room temperature and supplemental media prepared by adding 100× Hepes to the needed volume of growth media (DMEM, 10% FBS, beta-mercaptoethanol).

Viral cocktails are prepared such that an approximate MOI of ˜50 virus units per cell (˜5 for the transduction of c-Myc) can be conveniently applied to the wells. Cocktails include: (1) Oct4 and Sox2; (2) Oct4, Sox2 and c-Myc; or (3) Klf4, Oct4 and Sox2. The viral cocktails that are prepared should also contain: 0 μM DIM or di-Bromo-DIM, 1 μM DIM or di-Bromo-DIM, 5 μM DIM or di-Bromo-DIM, 10 μM DIM or di-Bromo-DIM, 20 μM DIM or di-Bromo-DIM, 30 μM DIM or di-Bromo-DIM, 50 μM DIM or di-Bromo-DIM, or 100 μM DIM or di-Bromo-DIM, as described herein. Each pipette that contacts virus is washed with bleach by drawing bleach solution to top of pipette and dispensing it back into the bleach bottle, followed by dispensing in an in-hood biohazard waste receptacle

Spent media is aspirated from the human fibroblast plate and polybrene is added to the viral cocktail (8 μg/mL final concentration), which is added onto wells of fibroblast cells. The plates are returned to the incubator and transduction allowed to proceed for about 18 to 36 hours, depending upon the viral stock, transgene, envelope and cell type.

Following transduction, viral cocktail is removed with a fresh 2 mL pipette per well of cells and dispensed into 100% bleach solution to inactivate. 2 mL of fresh hFIB media (containing 0 μM DIM or di-Bromo-DIM, 1 μM DIM or di-Bromo-DIM, 5 μM DIM or di-Bromo-DIM, 10 μM DIM or di-Bromo-DIM, 20 μM DIM or di-Bromo-DIM, 30 μM DIM or di-Bromo-DIM, 50 μM DIM or di-Bromo-DIM, or 100 μM DIM or di-Bromo-DIM) per well of cells is added and the plates replaced in the incubator for 48 hours. The media is thereafter replaced with hES cell media as described above. The hES cell media also contain: 0 μM DIM or di-Bromo-DIM, 1 μM DIM or di-Bromo-DIM, 5 μM DIM or di-Bromo-DIM, 10 μM DIM or di-Bromo-DIM, 20 μM DIM or di-Bromo-DIM, 30 μM DIM or di-Bromo-DIM, 50 μM DIM or di-Bromo-DIM, or 100 μM DIM or di-Bromo-DIM. Following media replacement, the cells are returned to the incubator. The media is replaced with fresh media daily, and each replacement also contains the corresponding level of DIM or di-Bromo-DIM. From the second feeding on, bleaching of the aspirating pipette is no longer necessary.

Colonies begin to appear approximately 2 weeks after viral transduction. About 1 month after viral transduction, colonies are picked and denoted Passage 0. The colonies are analyzed for pluripotency using any known method in the art, including but not limited to: testing for expression of pluripotency markers, as described herein; testing for long-term self-renewal capabilities, as described herein; or analyzing teratoma-forming ability, as described herein.

### Example 10

### Prophetic Example

About 40000 human fibroblasts per well are plated on a 6-well plate and grown in hFIB media (DMEM, 20% Earl's 199 media, 10% FBS, Glutamax, beta-mercaptoethanol (bME)) in the presence of TGF-beta receptor inhibitors such as A-83-01 or 616 453. TGF-beta receptor inhibitors are added at concentration of 0 μM, 1 μM, 5 μM, 10 μM, 20 μM, 30 μM, 50 μM or 100 μM, as described herein.

About 0 to 8 days after adding the TGF-beta receptor inhibitor, spent media is aspirated from human fibroblast plates which are then washed with 3 mL sterile PBS per well of a 6-well plate.

In a representative transduction protocol for the induction of pluripotency, viral stocks are thawed to room temperature and supplemental media prepared by adding 100× Hepes to the needed volume of growth media (DMEM, 10% FBS, beta-mercaptoethanol).

Viral cocktails are prepared such that an approximate MOI of ˜50 virus units per cell (˜5 for the transduction of c-Myc) can be conveniently applied to the wells. Cocktails include: (1) Oct4 alone (2) Klf4 and Oct4; or (3) Klf4, Oct4 and c-Myc. The viral cocktails that are prepared should also contain TGF-beta receptor inhibitors at concentration of 0 μM, 1 μM, 5 μM, 10 μM, 20 μM, 30 μM, 50 μM or 100 μM, as described herein. Each pipette that contacts virus is washed with bleach by drawing bleach solution to top of pipette and dispensing it back into the bleach bottle, followed by dispensing in an in-hood biohazard waste receptacle.

Spent media is aspirated from the human fibroblast plate and polybrene is added to the viral cocktail (8 μg/mL final concentration), which is added onto wells of fibroblast cells. The plates are returned to the incubator and transduction allowed to proceed for about 18 to 36 hours, depending upon the viral stock, transgene, envelope and cell type.

Following transduction, viral cocktail is removed with a fresh 2 mL pipette per well of cells and dispensed into 100% bleach solution to inactivate. 2 mL of fresh hFIB media (containing TGF-beta receptor inhibitors at concentration of 0 μM, 1 μM, 5 μM, 10 μM, 20 μM, 30 μM, 50 μM or 100 μM) per well of cells is added and the plates replaced in the incubator for 48 hours. The media is thereafter replaced with hES cell media as described above. The hES cell media also contain TGF-beta receptor inhibitors at concentration of 0 μM, 1 μM, 5 μM, 10 μM, 20 μM, 30 μM, 50 μM or 100 μM. Following media replacement, the cells are returned to the incubator. The media is replaced with fresh media daily, and each replacement also contains the corresponding level of DIM or di-Bromo-DIM. From the second feeding on, bleaching of the aspirating pipette is no longer necessary.

Colonies begin to appear approximately 2 weeks after viral transduction. About 1 month after viral transduction, colonies are picked and denoted Passage 0. The colonies are analyzed for pluripotency using any known method in the art, including but not limited to: testing for expression of pluripotency markers, as described herein; testing for long-term self-renewal capabilities, as described herein; or analyzing teratoma-forming ability, as described herein.

### Example 11

### Enhanced Efficiency of Three Factor and Four Factor Reprogramming by TGF-βR Inhibitors

In order to determine the effect of TGFβR inhibitor compounds on reprogramming we combined low multiplicity of infection (MOI) of retroviruses for three induction factor (Klf4, Sox2, and Oct ¾ or “KSO”) and four induction factor (Klf4, Sox2, Oct3/4, c-Myc or “KSOM”) reprogramming with various TGFβR inhibitors in human BJ-5Tα fibroblasts or BJ fibroblasts. BJ-5Tα fibroblasts express TERT and exhibit accelerated reprogramming kinetics amenable to high-throughput screening assays. By utilizing a low MOI infection protocol, we increased the sensitivity of the assay to detect the ability of various TGFβR inhibitor compounds to enhance the efficiency of reprogramming by three or four induction factor combinations. Alkaline phosphatase is expressed in pluripotent stem cells, and is considered to be an early marker of cellular reprogramming, moreover its enzymatic activity is amenable to quantitation and high-throughput assay of compounds for their ability to enhance reprogramming and/or substitute for induction/reprogramming factors.

Adherent BJ or BJ-5Tα (TERT-immortalized) fibroblast cells were infected “in bulk” with retroviruses for expression of the three induction factors (also referred to as “reprogramming factors”) KLF4, OCT3/4 and SOX2 (“KSO”), or the cells were infected with retroviruses for expression of KLF4, OCT4, SOX2 and c-MYC (“KSOM”), in each case at a multiplicity of infection (MOI) of 1, and transferred into 96-well plates in a serum-free human embryonic stem cell (hES) medium. On the following day (Day “0”), TGFβ inhibitor compounds or DMSO (negative control vehicle) were added to the cultures in 12-point serial dilution fashion using an automated workstation. Medium and compounds were refreshed at Day 4. On Day 8 the TGFβR inhibitor compound-treated and DMSO-treated cells were lysed and assayed for alkaline phosphatase activity (AP) activity. AP activity was determined quantitatively using a chemiluminescent 1,2-dioxetane-phosphate substrate, which upon dephosphorylation by AP forms a metastable phenolate anion intermediate that decomposes emitting light quantified by a microplate luminometer.

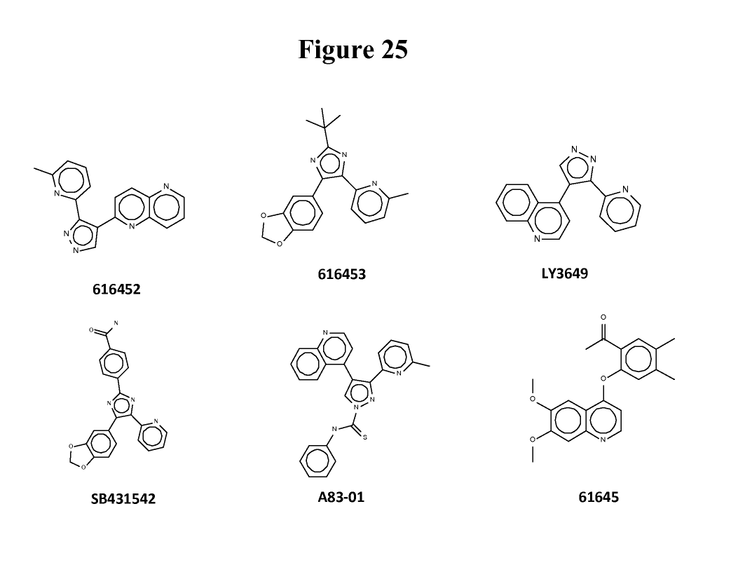

The TGFβR inhibitor compounds as shown in FIG. 25 were tested for their ability to increase alkaline phosphatase gene expression/activity.

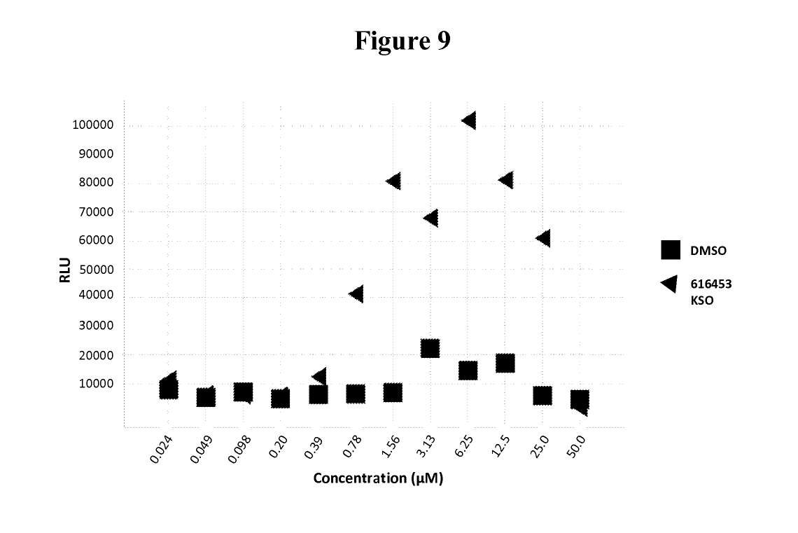

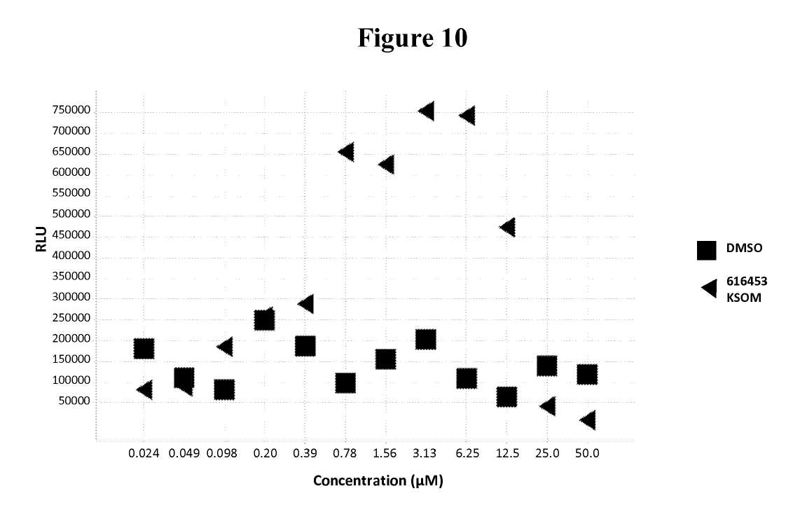

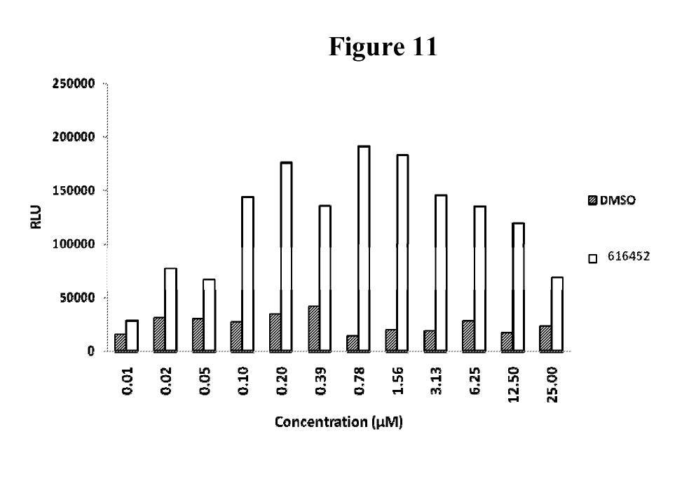

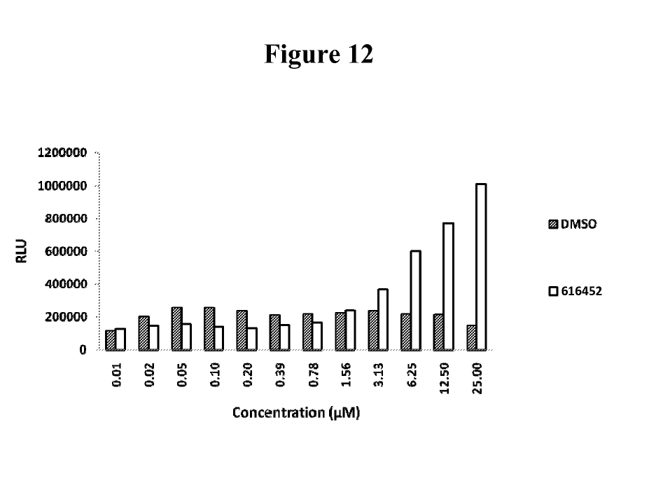

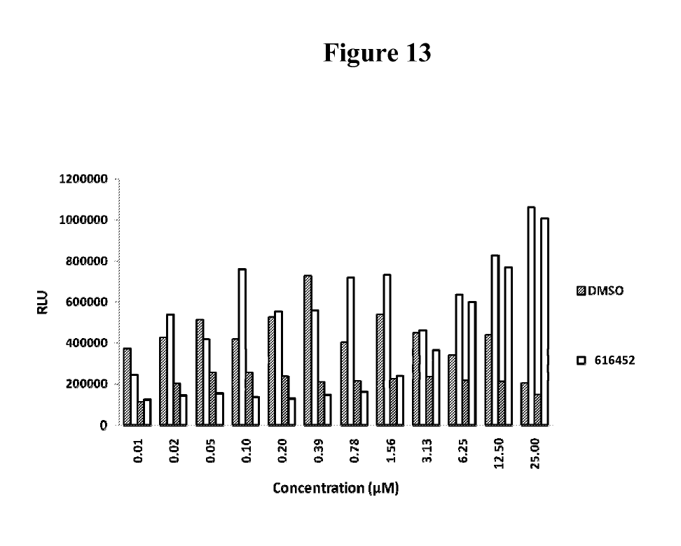

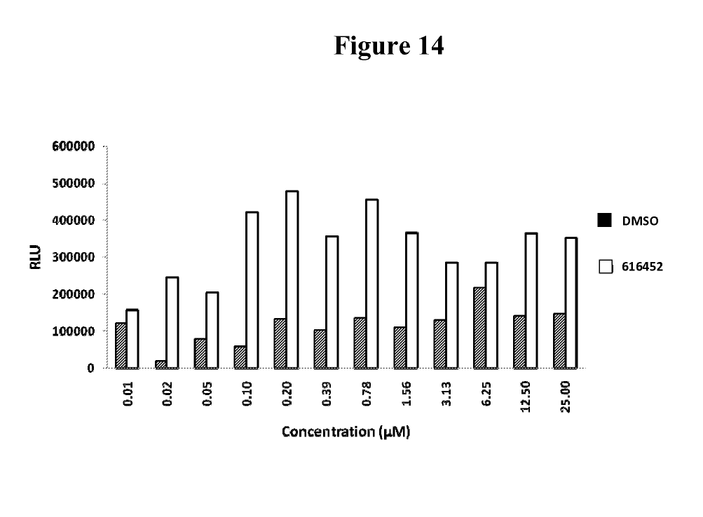

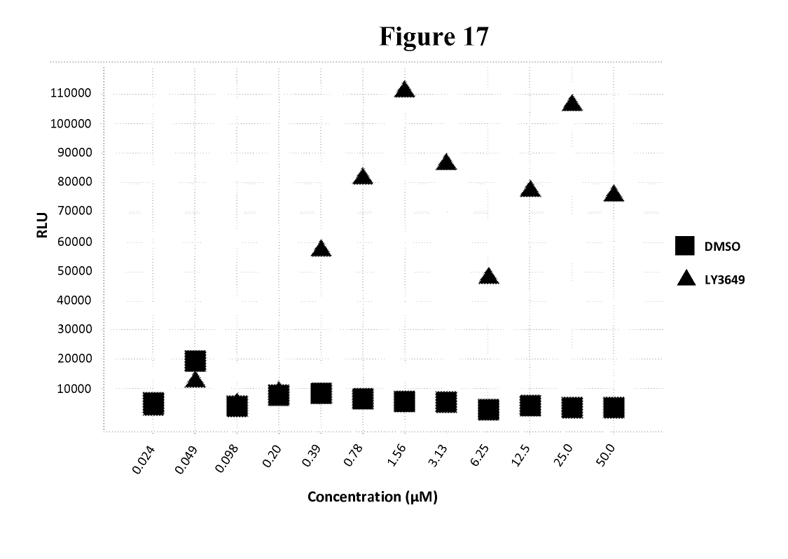

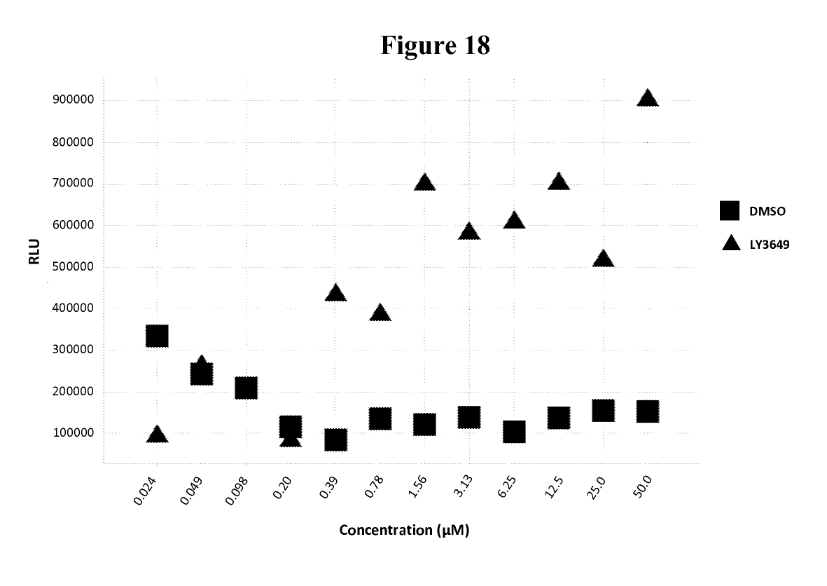

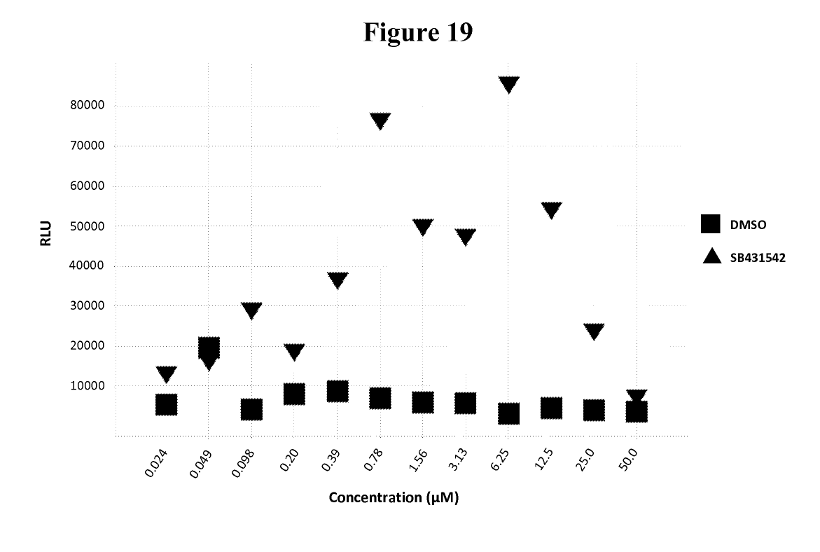

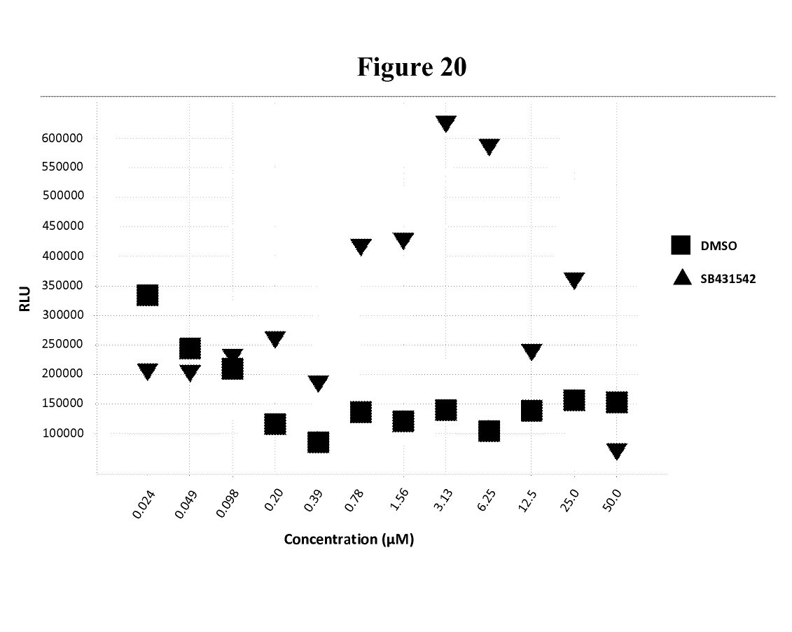

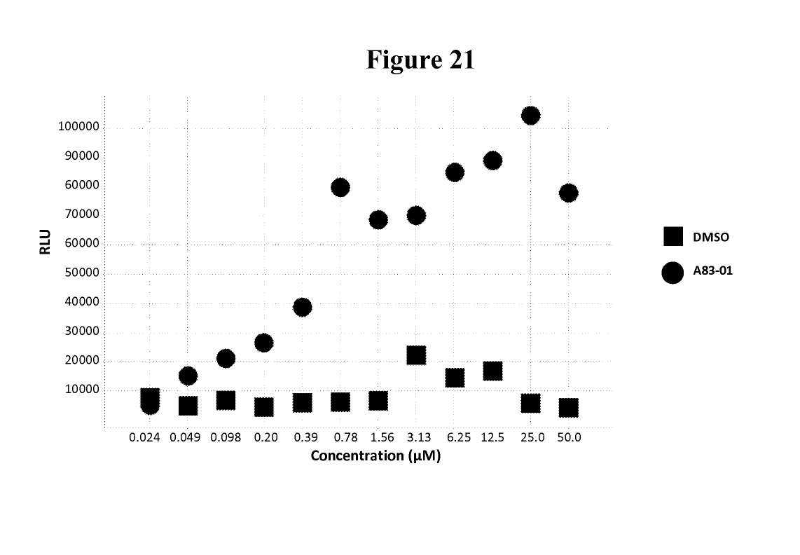

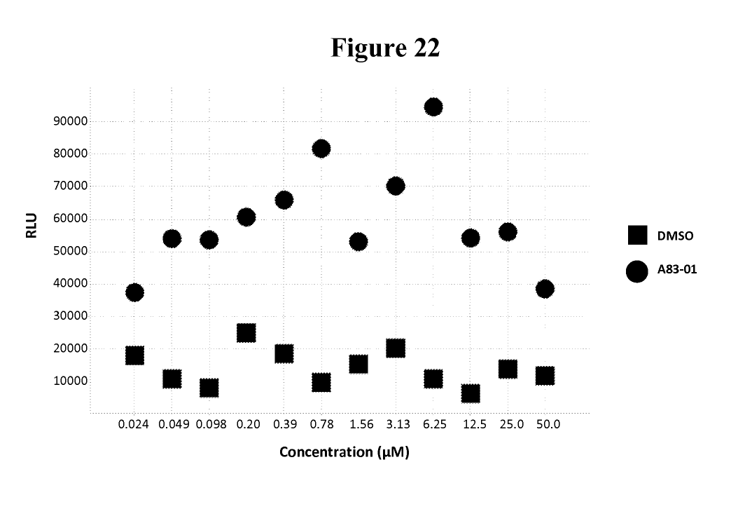

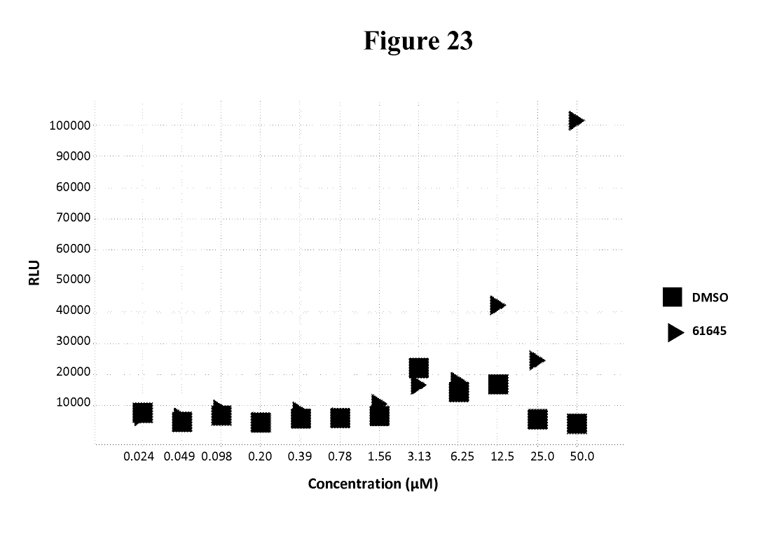

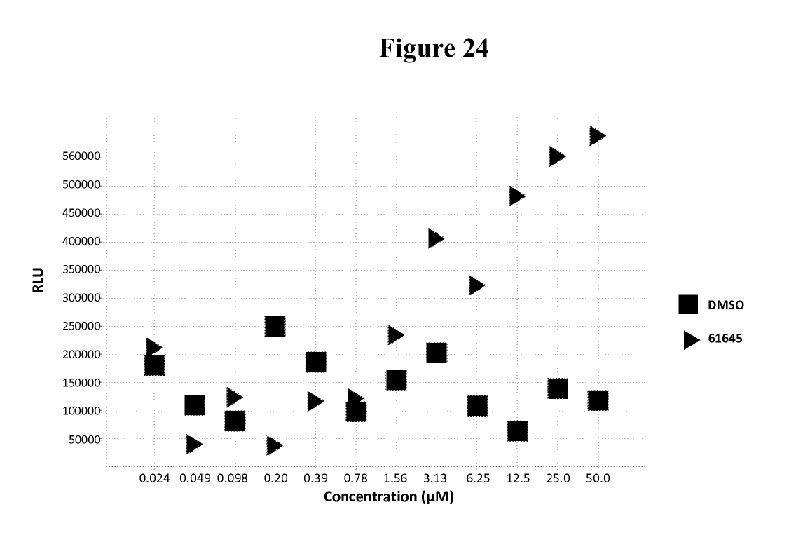

Assay results are shown in the figures as follows: 616453 (see FIGS. 9-10), 616452 (see FIGS. 11-16), LY (see FIGS. 17-18), SB431542 (see FIGS. 19-20), A83-01 (see FIGS. 21-22), and 61645 (see FIGS. 22-24). Of note, 616452 was also tested in human BJ fibroblasts (that lack TERT), and, as shown, in FIGS. 12 and 13, this compound also increased alkaline phosphatase activity in both the three and four factor reprogramming protocols, albeit, with a reduced sensitivity compared to the BJ-5Tα fibroblasts.

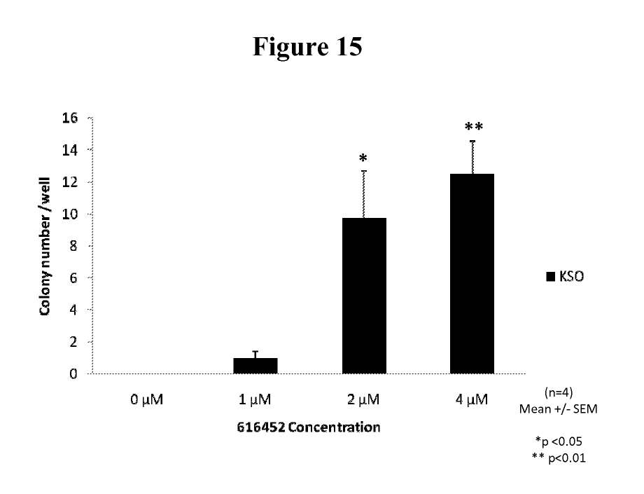

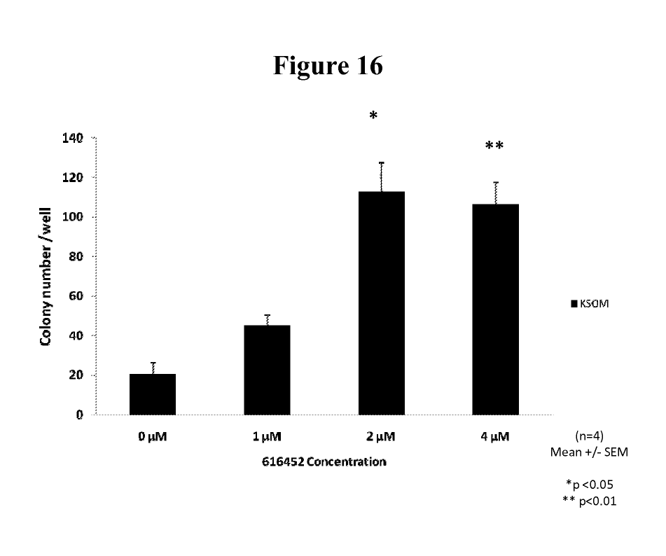

In the case of compound 616452, cells KSO and KSOM-infected BJ-5Tα fibroblasts were fixed by 4% paraformaldehyde at Day 8, and then incubated with 50 μl 1-Step NBT/BCIP for 20 minutes for AP staining AP staining-positive colonies were counted (see FIGS. 15-16).

Fibroblasts treated with compounds showed increased AP signal by up to 10 fold, compared to that without compound treatment. Consistent with this, the number of AP positive colonies also increased dramatically in human BJ-5Tα fibroblasts when KSO or KSOM-infected cells were treated with 616452 (KSO: greater than ten fold; KSOM: approximately five fold).

Based on these data we concluded that TGFβR inhibitors, e.g., those having a structural formula corresponding to Formula (II), Formula (III), or Formula (IV) enhance the efficiency of human cellular reprogramming by the forced expression of the induction factors KSO or KSOM.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of increasing the potency of a human cell comprising contacting a plurality of human cells with a TGF-β receptor (TGFβR) inhibitor and forcing expression of one or more induction factors to obtain a plurality of human cells having increased potency.

2. The method of claim 1, wherein the plurality of human cells having increased potency have increased expression of one or more markers of pluripotency.

3. The method of claim 2, wherein the one or more markers of pluripotency comprise alkaline phosphatase, SSEA-3, SSEA-4, TRA-1-60, TRA-1-81, Nanog, Oct-3/4, Sox2, GDF3, REX1, FGF4, ESG1, DPPA2, DPPA4, and hTERT.

4. The method of claim 3, wherein the one or markers of pluripotency comprise alkaline phosphatase.

5. The method of claim 1, wherein the TGFβR inhibitor is a compound having the structure of Formula (II), Formula (III), or Formula (IV): 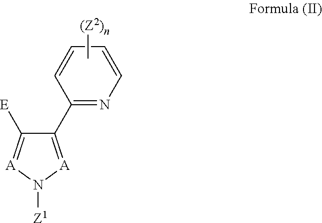
wherein,
E is selected from 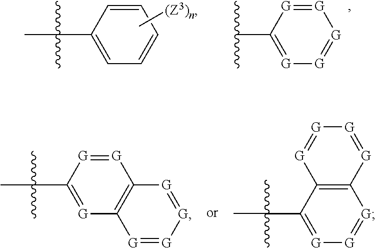
G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is H, CONHAr, or CSNHAr;
Ar is 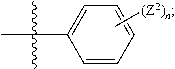
n is 0, 1, 2 or 3; and
each Z² is independently a C₁-C₈ straight, branched or cyclic hydrocarbon group, a C₁-C₆ alkoxy group, or, optionally, two Z² groups, together with the carbon atoms to which said Z² groups are attached, combine to form a cyclic group; 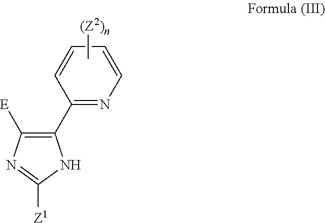
wherein
E is selected from 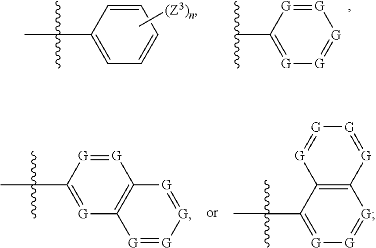
G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is a C₁-C₈ straight, branched, or cyclic hydrocarbon group, or an Ar group;
Ar is 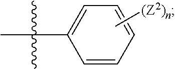
Z² is a C₁-C₈ straight, branched, or cyclic hydrocarbon group, a C₁-C₆ alkoxy group, a —CONH₂ group, or a —CSNH₂ group; and
each n is independently 0, 1, 2, or 3; or 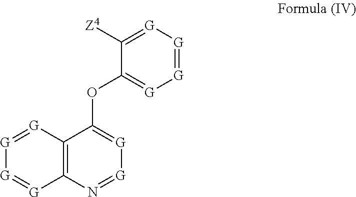
wherein
G is N, CH or CZ²;
Z² is a C₁-C₈ straight, branched, or cyclic hydrocarbon group, or a C₁-C₆ alkoxy group;
Z⁴ is a COZ² group, a CON(R⁵)₂ group; and
each Z⁵ is independently a hydrogen or C₁-C₈ straight, branched, or cyclic hydrocarbon group.

6. The method of claim 5, wherein the compound of Formula (II) has the structure of Formula (IIa): 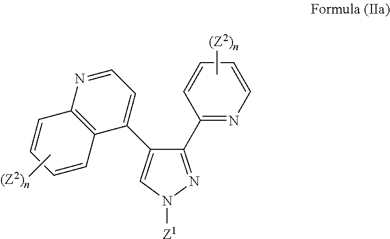
wherein:
Z¹ is H, CONHAr, or CSNHAr;
Z² is a C₁-C₈ straight, branched, or cyclic hydrocarbon group;
Ar is 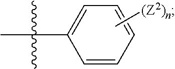
and
each n is independently 0, 1, 2, or 3.

7. The method of claim 6, wherein the compound of Formula (IIa) has the structure:

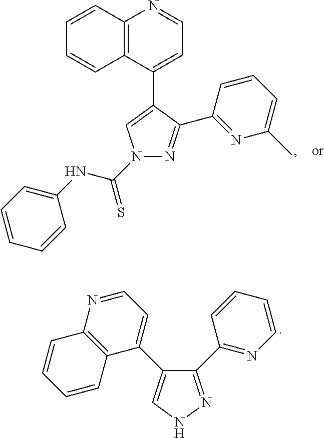

8. The method of claim 5, wherein the compound of Formula (II) has the structure of Formula (IIb): 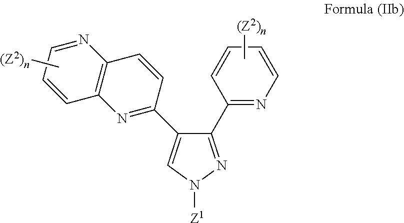
wherein:
Z¹ is H, CONHAr, or CSNHAr;
Z² is a C₁-C₈ straight, branched, or cyclic hydrocarbon group;
Ar is 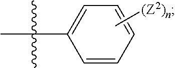
and
each n is independently 0, 1, 2, or 3.

9. The method of claim 8, wherein the compound of Formula (IIb) has the structure:

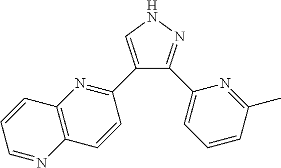

10. (canceled)

11. The method of claim 5, wherein the compound of Formula (III) has the structure of Formula (IIIa): 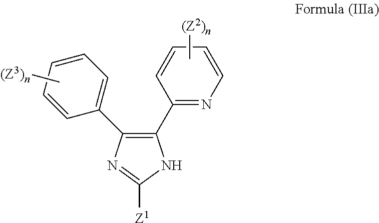
wherein,
Z¹ is C₁-C₈ straight, branched, or cyclic hydrocarbon group, or an Ar group;
Z² is a C₁-C₈ straight, branched, or cyclic hydrocarbon group, a C₁-C₆ alkoxy group, a —CONH₂ group, or a —CSNH₂ group;
Z³ is a C₁-C₈ straight, branched, or cyclic hydrocarbon group, a C₁-C₆ alkoxy group, or two Z³ groups together form a —OCH₂O— or —OCH₂CH₂O— group;
Ar is 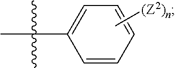
and
each n is independently 0, 1, 2, or 3.

12. The method of claim 11, wherein the compound of Formula (IIIa) has the structure:

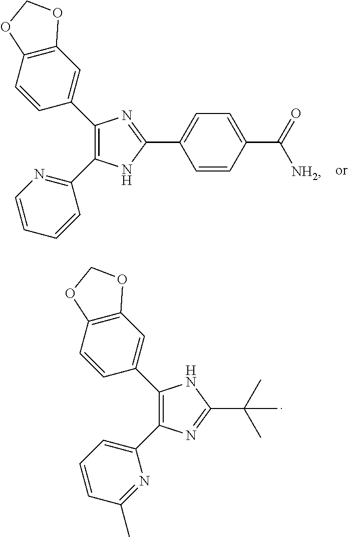

13. (canceled)

14. The method of claim 5, wherein the compound of Formula (IV) has the structure of Formula (IVa): 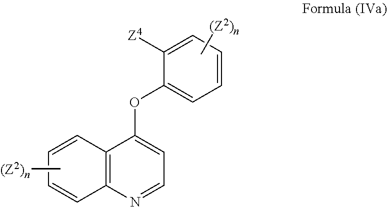
wherein,
Z² is a C₁-C₈ straight, branched, or cyclic hydrocarbon group, a C₁-C₆ alkoxy group;
Z⁴ is a COCH₃ group, a CONH2 group, a CONH(CH₃) group; and
each n is independently 0, 1, 2, or 3.

15. The method of claim 14, wherein the compound of Formula (IVa) has the structure:

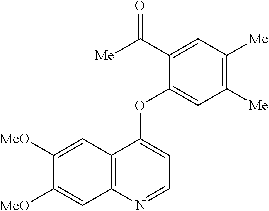

16. The method of claim 2, wherein the enhanced expression is a greater than 2-fold increase in RNA expression compared to cells that have not been contacted with the chemical compound.

17. (canceled)

18. The method of claim 1, further comprising forcing the expression in the plurality of human cells of one or more of the following induction factors: Oct3/4, Sox2, Klf4, c-Myc, Lin28, or Nanog.

19.-20. (canceled)

21. The method of claim 1, further comprising contacting the plurality of human cells with one or more of the following agents: DNA demethylating agent, histone methyltransferase inhibitor, histone deacetylase (HDAC) inhibitor, L-type calcium channel agonist, Wnt ligand, siRNA against p53, siRNA against Utf1 cDNA.

22. The method of claim 1, wherein said the plurality of human cells comprise fibroblasts, blood cells, keratinocytes, hair follicle cells, or epithelial cells.

23. The methods of claim 1, wherein said the plurality of human cells are derived from a patient.

24. The method of claim 23, wherein the patient is suffering from a neurodegenerative disease or disorder, a hepatic injury, or diabetes.

25. The method of claim 24, wherein the neurodegenerative disease or disorder is Alzheimer's Disease or Parkinson's Disease.

26.-27. (canceled)

28. A method for generating human induced pluripotent stem cells comprising contacting primary cells obtained from a human subject with a small molecule TGFβ receptor (TGFβR) inhibitor compound and forcing expression of one or more induction factors in the primary cells from the human subject to obtain the human induced pluripotent stem cells.

29. The method of claim 28, wherein the small molecule TGFβR inhibitor compound is a compound having the structure of Formula (II), Formula (III), or Formula (IV): 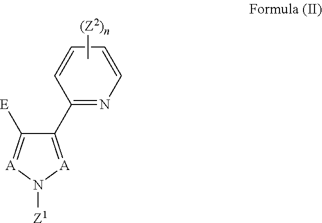
wherein,
E is selected from 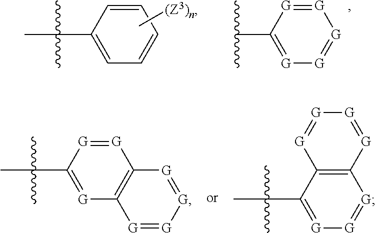
G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is H, CONHAr, or CSNHAr;
Ar is 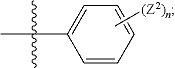
n is 0, 1, 2 or 3; and
each Z² is independently a C₁-C₈ straight, branched or cyclic hydrocarbon group, a C₁-C₆ alkoxy group, or, optionally, two Z² groups, together with the carbon atoms to which said Z² groups are attached, combine to form a cyclic group; 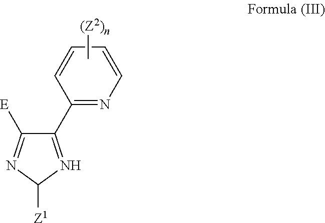
wherein
E is selected from 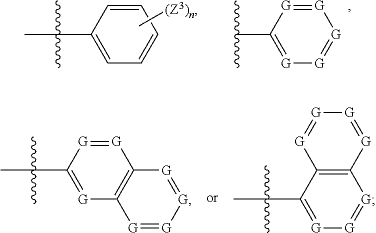
G is N, O, CH or CZ³, wherein at least one G is N;
each Z³ is independently a C₁-C₈ straight, branched, or cyclic hydrocarbon group, a C₁-C₆ alkoxy group, or, optionally, two Z³ groups, together with the carbon atoms to which the Z³ groups are attached, combine to form a cyclic group;
Z¹ is a C₁-C₈ straight, branched, or cyclic hydrocarbon group, or an Ar group;
Ar is 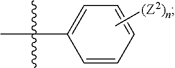
Z² is a C₁-C₈ straight, branched, or cyclic hydrocarbon group, a C₁-C₆ alkoxy group, a —CONH₂ group, or a —CSNH₂ group; and
each n is independently 0, 1, 2, or 3; or 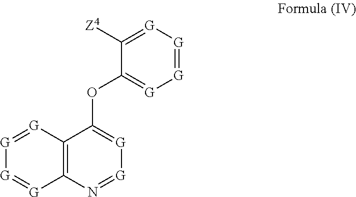
wherein,
G is N, CH or CZ²;
Z is a C₁-C₈ straight, branched, or cyclic hydrocarbon group, or a C₁-C₆ alkoxy group;
Z⁴ is a COZ² group, a CON(R⁵) group; and
each Z⁵ is independently a hydrogen or C₁-C₈ straight, branched, or cyclic hydrocarbon group.

30.-31. (canceled)
